(19) United States
(12) Patent Application Publication
(10) Pub. No.: US 2022/0088049 A1
(43) Pub. Date: Mar. 24, 2022

(54) COMPOSITIONS, METHODS, AND KITS FOR DELIVERY OF POLYRIBONUCLEOTIDES

(71) Applicant: Flagship Pioneering Innovations VI, LLC, Cambridge, MA (US)

(72) Inventors: Avak KAHVEJIAN, Lexington, MA (US); Nicholas McCartney PLUGIS, Duxbury, MA (US); Alexandra Sophie DE BOER, Somerville, MA (US); Ellese Marie CARMONA, Boston, MA (US); Morag Helen STEWART, Boston, MA (US); Roger Joseph HAJJAR, Lexington, MA (US)

(21) Appl. No.: 17/433,658

(22) PCT Filed: Mar. 1, 2020
(86) PCT No.: PCT/US2020/020560

§ 371 Date: Aug. 25, 2021

Related U.S. Application Data
(60) Provisional application No. 62/812,763, filed on Mar. 1, 2019.

(51) Int. Cl. A61K 31/7088; A61K 47/10

(52) U.S. Cl. CPC A61K 31/7088; A61K 47/10

(57) ABSTRACT

Disclosed herein are methods and compositions for delivery of a polyribonucleotide. In some cases, the methods and compositions provided herein are suitable for in vivo delivery of polyribonucleotides. In certain aspects, also disclosed herein are pharmaceutical compositions for delivery of polyribonucleotide having biological effects.

### CROSS-REFERENCE TO RELATED APPLICATIONS

The present application claims priority to and benefit from U.S. Provisional Application No. 62/812,763, filed Mar. 1, 2019, the entire contents of which is herein incorporated by reference.

### BACKGROUND

Polyribonucleotides are critical biomolecules for biological activities such as gene expression, gene regulation, and cellular signaling transduction.

### SUMMARY

The present invention described herein includes compositions, pharmaceutical compositions, and methods for delivery of polyribonucleotides. In certain embodiments, the compositions and pharmaceutical compositions include ethanol and the polyribonucleotides. In other embodiments, the compositions and pharmaceutical compositions include alcohol and the polyribonucleotides. In another embodiment, the compositions and pharmaceutical compositions include a cell-penetrating agent and the polyribonucleotides. The methods comprise applying these compositions and pharmaceutical compositions to a surface area of a subject.

The present invention described herein includes compositions free of any carrier and comprises a polyribonucleotide and diluent. The compositions can be applied to epithelial cells for delivery of the polyribonucleotide. The compositions can be applied to a surface area after application of a sterilizing agent to that area.

In some aspects, a pharmaceutical composition comprises a mixture of a polyribonucleotide and ethanol, wherein the ethanol constitutes at least about 0.3% v/v to about 75% v/v of the mixture. In some embodiments, the ethanol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture. In some embodiments, the ethanol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

In some aspects, a pharmaceutical composition comprises a mixture of a polyribonucleotide and an alcohol, wherein the alcohol constitutes at least about 0.3% v/v to about 75% v/v of the mixture. In some embodiments, the alcohol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture. In some embodiments, the alcohol constitutes at least about at 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

In some aspects, a pharmaceutical composition comprises a mixture of a polyribonucleotide and a cell-penetrating agent, wherein the cell-penetrating agent constitutes at least about 0.3% v/v to about 75% v/v of the mixture. In some embodiments, the cell-penetrating agent constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture. In some embodiments, the cell-penetrating agent constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture. In some embodiments, the cell-penetrating agent is an alcohol. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

In some embodiments, the polyribonucleotide encodes a protein. In some embodiments, the protein is a therapeutic protein. In some embodiments, the protein is a wound healing protein. In some embodiments, the wound healing protein is a growth factor. In some embodiments, the growth factor is EGF, PDGF, TGFβ, or VEGF. In some embodiments, the pharmaceutical composition is a liquid, gel, lotion, paste, cream, foam, or stick. In some embodiments, the polyribonucleotide is a linear polyribonucleotide. In some embodiments, the polyribonucleotide is an mRNA. In some embodiments, the polyribonucleotide lacks a cap or poly-A tail. In some embodiments, the polyribonucleotide is immunogenic. In some embodiments, the polyribonucleotide is non-immunogenic. In some embodiments, the polyribonucleotide is a circular polyribonucleotide. In some embodiments, the polyribonucleotide comprises a modified ribonucleotide. In some embodiments, the pharmaceutical composition has a pH of about 7. In some embodiments, the pharmaceutical composition has a viscosity that is about the same as water. In some embodiments, the pharmaceutical composition is substantially free of hydrophobic or lipophilic groups. In some embodiments, the pharmaceutical composition is substantially free of hydrocarbons. In some embodiments, the pharmaceutical composition is substantially free of cationic liposomes. In some embodiments, the pharmaceutical composition is substantially free of fatty acids, lipids, liposomes, cholesterol, or any combination thereof. In some embodiments, the cell penetrating agent is soluble in polar solvents. In some embodiments, the cell penetrating agent is insoluble in polar solvents.

In some aspects, a therapeutic composition comprises a polyribonucleotide and a cell-penetrating agent, wherein the cell-penetrating agent is configured for topical administration. In some aspects, a therapeutic composition comprises a polyribonucleotide and an alcohol, wherein the alcohol is configured for topical administration.

In some aspects, a therapeutic composition comprises a polyribonucleotide and an alcohol, wherein the polyribonucleotide comprises a payload or a sequence encoding a payload and wherein the payload has a biological effect on a cell. In some aspects, a therapeutic composition comprises a polyribonucleotide and a cell-penetrating agent, wherein the polyribonucleotide comprises a payload or a sequence encoding a payload and wherein the payload has a biological effect on a cell.

In some aspects, a therapeutic composition comprises a polyribonucleotide and an alcohol, wherein the polyribonucleotide is in an amount effective to have a biological effect on a cell or tissue and wherein the alcohol is in an amount effective to have a biological effect on a cell or tissue. In some aspects, a therapeutic composition comprises a polyribonucleotide and a cell-penetrating agent, wherein the polyribonucleotide is in an amount effective to have a biological effect on a cell or tissue and wherein the cell-penetrating agent is in an amount effective to have a biological effect on a cell or tissue.

In some aspects, a method of treating a wound comprises contacting the wound or tissue surrounding the wound to a composition comprising a mixture of a polyribonucleotide and ethanol, wherein the ethanol constitutes at least about 0.3% v/v to about 75% v/v of the mixture. In some aspects, a method of treating a wound comprises contacting the wound or tissue surrounding the wound to a composition comprising a mixture of a polyribonucleotide and alcohol, wherein the alcohol constitutes at least about 0.3% v/v to about 75% v/v of the mixture. In some aspects, a method of treating a wound comprises contacting the wound or tissue surrounding the wound to a composition comprising a mixture of a polyribonucleotide and a cell-penetrating agent, wherein the cell-penetrating agent constitutes at least about 0.3% v/v to about 75% v/v of the mixture.

In some aspects, a therapeutic composition comprises a polyribonucleotide, an alcohol, and a topical delivery excipient, wherein the topical delivery excipient comprises a stabilizer. In some aspects, a therapeutic composition comprises a polyribonucleotide, a cell-penetrating agent, and a topical delivery excipient, wherein the topical delivery excipient comprises a stabilizer. In some embodiments, the stabilizer comprises glucose (4.5 g/L).

In some aspects, a suppository or other lipid based formulation comprising a polyribonucleotide and an alcohol. In some aspects, a suppository or other lipid based formulation comprising a polyribonucleotide and a cell-penetrating agent.

In some aspects, an inhalable composition comprising a mixture of a polyribonucleotide, an alcohol, and a propellant. In some aspects, an inhalable composition comprising a mixture of a polyribonucleotide, a cell-penetrating agent, and a propellant.

In some aspects, a therapeutic composition comprises a biodegradable scaffold loaded with polyribonucleotide and an alcohol. In some aspects, a therapeutic composition comprises a biodegradable scaffold loaded with polyribonucleotide and a cell-penetrating agent.

In some embodiments, the cell-penetrating agent comprises an alcohol. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol is ethanol.

In some aspects, a method of delivering a polyribonucleotide to a subject comprises: a) applying a sterilizing agent to a surface area of the subject; b) applying a composition free of any carrier comprising the polyribonucleotide and diluent to the surface area. In some embodiments, the sterilizing agent is an alcohol, UV light, laser light, or heat.

In some aspects, a method of delivering a polyribonucleotide to a subject comprises: a) applying an alcohol to a surface area of the subject; b) applying a composition free of any carrier comprising the polyribonucleotide and diluent to the surface.

In some aspects, a method of delivering a polyribonucleotide to an epithelial cell comprises applying a composition free of any carrier comprising a diluent and a polyribonucleotide that is not modified to the epithelial cell.

In some aspects, a method of delivering a polyribonucleotide to a subject comprises topically applying a composition comprising a mixture of a polyribonucleotide and ethanol to a surface area of the subject, wherein the ethanol constitutes at least about 0.3% v/v to about 75% v/v of the mixture. In some aspects, a method of delivering a polyribonucleotide to a subject comprises topically applying a composition comprising a mixture of a polyribonucleotide and an alcohol to a surface area of the subject, wherein the alcohol constitutes at least about 0.3% v/v to about 75% v/v of the mixture. In some aspects, a method of delivering a polyriboribonucleotide to a subject comprises topically applying a composition comprising a mixture of a polyribonucleotide and a cell-penetrating agent to a surface area of the subject, wherein the cell-penetrating agent constitutes at least about 0.3% v/v to about 75% v/v of the mixture. In some embodiments, the composition delivers the polyribonucleotide to a dermal or epidermal tissue of the subject. In some embodiments, the composition delivers the polyribonucleotide to the dermal or epidermal tissue of the subject without iontophoresis.

In some aspects, a method of delivering a polyribonucleotide to a cell or tissue comprises contacting the cell or tissue to a mixture comprising the polyribonucleotide and alcohol, wherein the alcohol constitutes at least about 0.3% v/v to about 75% v/v of the mixture. In some aspects, a method of delivering a polyribonucleotide to a cell or tissue comprises contacting the cell or tissue to a mixture comprising the polyribonucleotide and a cell-penetrating agent, wherein the cell-penetrating agent constitutes at least about 0.3% v/v to about 75% v/v of the mixture.

In some aspects, a method of delivering a therapeutic composition to a cell or tissue comprises contacting the cell or tissue to the therapeutic composition comprising a polyribonucleotide and an alcohol, wherein the alcohol is configured for topical administration. In some aspects, a method of delivering a therapeutic composition to a cell or tissue comprises contacting the cell or tissue to the therapeutic composition comprising a polyribonucleotide and a cell-penetrating agent, wherein the cell-penetrating agent is configured for topical administration.

In some aspects, a method of in vivo delivery of a polyribonucleotide comprises applying a mixture comprises the polyribonucleotide and an alcohol onto a surface area of a subject. In some aspects, a method of in vivo delivery of a polyribonucleotide comprises applying a mixture comprising the polyribonucleotide and a cell-penetrating agent onto a surface area of a subject.

In some aspects, a method of topical delivery of a polyribonucleotide comprises applying a mixture comprising the polyribonucleotide and an alcohol onto a surface area of a subject. In some aspects, a method of topical delivery of a polyribonucleotide comprises applying a mixture comprising the polyribonucleotide and a cell-penetrating agent onto a surface area of a subject.

In some aspects, a method of delivering a therapeutic polyribonucleotide to a subject comprises topically contacting a mixture comprising the therapeutic polyribonucleotide and an alcohol to an epithelial surface, endothelial surface, exposed tissue, or open wound. In some aspects, a method of delivering a therapeutic polyribonucleotide to a subject comprises topically contacting a mixture comprising the therapeutic polyribonucleotide and a cell-penetrating agent to an epithelial surface, endothelial surface, exposed tissue, or open wound.

In some aspects, a method of treatment comprises applying a mixture comprises a polyribonucleotide and an alcohol to a surface area of a subject with a condition or disease. In some aspects, a method of treatment comprises applying a mixture comprising a polyribonucleotide and a cell-penetrating agent to a surface area of a subject with a condition or disease. In some embodiments, the cell-penetrating agent comprises an alcohol. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol comprises ethanol. In some embodiments, the ethanol, alcohol, or cell-penetrating agent constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture. In some embodiments, the ethanol, alcohol, or cell-penetrating agent at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture. In some embodiments, the method further comprises mixing the polyribonucleotide with the cell-penetrating agent or alcohol. In some embodiments, the polyribonucleotide is in a solid form before the mixing. In some embodiments, the polyribonucleotide is lyophilized before the mixing. In some embodiments, the polyribonucleotide is in a liquid form before the mixing. In some embodiments, the polyribonucleotide is dissolved in a solvent before the mixing. In some embodiments, the polyribonucleotide comprises a payload or a sequence encoding a payload and wherein the payload has a biological effect on a cell or a tissue. In some embodiments, the polyribonucleotide is in an amount effective to have a biological effect on a cell and the cell-penetrating agent is in an amount effective to have a biological effect on a cell or a tissue. In some embodiments, the polyribonucleotide encodes a protein. In some embodiments, the protein is a therapeutic protein. In some embodiments, the protein is a wound healing protein. In some embodiments, the wound healing protein is a growth factor. In some embodiments, the growth factor is EGF, PDGF, TGFβ, or VEGF. In some embodiments, the composition is a liquid, gel, lotion, paste, cream, foam, or stick. In some embodiments, the polyribonucleotide is a linear polyribonucleotide. In some embodiments, the polyribonucleotide is an mRNA. In some embodiments, the polyribonucleotide lacks a cap or poly-A tail. In some embodiments, the polyribonucleotide is immunogenic. In some embodiments, the polyribonucleotide is non-immunogenic. In some embodiments, the polyribonucleotide is a circular polyribonucleotide. In some embodiments, the polyribonucleotide comprises a modified ribonucleotide. In some embodiments, the composition has a pH of about 7. In some embodiments, the composition has a viscosity that is about the same as water. In some embodiments, the composition is substantially free of hydrophobic or lipophilic groups. In some embodiments, the composition is substantially free of hydrocarbons. In some embodiments, the composition is substantially free of cationic liposomes. In some embodiments, the composition is substantially free of fatty acids, lipids, liposomes, cholesterol, or any combination thereof. In some embodiments, the cell penetrating agent is soluble in polar solvents. In some embodiments, the cell penetrating agent is insoluble in polar solvents. In some embodiments, the composition further comprises a pharmaceutically acceptable excipient. In some embodiments, the delivery is systemic. In some embodiments, the delivery is localized. In some embodiments, the surface area is selected from the group consisting of: skin, surface areas of oral cavity, nasal cavity, ear cavity, gastrointestinal tract, respiratory tract, vaginal, cervical, inter uterine, urinary tract, and eye. Where the surface area is of the oral cavity, nasal cavity, gastrointestinal tract, respiratory tract, vaginal, cervical, inter uterine, urinary tract, and eye, delivery is to the epithelial lining of such surface area though a non-invasive means. In some embodiments, applying comprises depositing a drop of the mixture directly onto the surface area. In some embodiments, applying comprises wiping the surface area with a patch, a gel, or a film embedded with the mixture. In some embodiments, applying comprises spraying the mixture onto the surface area. In some embodiments, applying comprises administering the mixture to the subject via aerosolization. In some embodiments, applying comprises administering the mixture to the subject via a suppository. In some embodiments, applying comprises administering the mixture to the subject via oral ingestion of a capsule containing the mixture, and wherein the capsule is configured to release the mixture inside gastrointestinal tract of the subject. In some embodiments, the cell comprises an epithelial cell. In some embodiments, the circular polyribonucleotide has a translation efficiency at least 5%, at least 10%, at least 15%, at least 20%, at least 30%, at least 40%, at least 50%, at least 60%, at least 70%, at least 80%, at least 90%, at least 100%, at least 150%, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold greater than a linear counterpart. In some embodiments, the circular polyribonucleotide has a translation efficiency at least 5 fold greater than a linear counterpart. In some embodiments, the polyribonucleotide has a short term biological effect.

In some embodiments, the polyribonucleotide has a long term biological effect. In some embodiments, a concentration of the polyribonucleotide in the mixture is at least about 50 ng/mL, at least about 100 ng/mL, at least about 500 ng/mL, at least about 1 μg/mL, at least about 2 μg/mL, at least about 3 μg/mL, at least about 4 μg/mL, at least about 5 μg/mL, at least about 10 μg/mL, at least about 20 μg/mL, at least about 50 μg/mL, at least about 100 μg/mL, at least about 200 μg/mL, at least about 500 μg/mL, at least about 1 mg/mL, at least about 2 mg/mL, at least about 5 mg/mL, at least about 10 mg/mL, at least about 20 mg/mL, at least about 50 mg/mL, or at least about 100 mg/mL.

The present invention also provides kits comprising a pharmaceutical composition described herein. In some aspects, a kit comprises an application tool and the pharmaceutical composition of any one the preceeding embodiments, wherein the application tool is configured to apply the pharmaceutical composition to a surface area of a subject.

In some aspects, a kit comprises a first application tool, a second application tool, a sterilizing agent, and a composition free of any carrier comprising the polyribonucleotide and diluent, wherein the first application tool is configured to apply a sterilizing agent to a surface area of a subject and the second application tool is configured to apply the composition to the surface area of the subject. In some embodiments, the sterilizing agent is an alcohol, UV light, laser light, or heat. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the first application tool is a wipe. In some embodiments, the wipe comprises the sterilizing agent. In some embodiments, first application tool is a device that applies UV light or laser light. In some embodiments, the first application tool is a device that applies heat.

In some aspects, a kit comprising an application tool and a mixture comprising a polyribonucleotide and a cell-penetrating agent, wherein the application tool is configured to apply the mixture to a surface area of a subject. In some embodiments, the application tool or second application tool comprises a pipette. In some embodiments, the application tool or second application tool comprises a substrate, and wherein the substrate is embedded with the mixture. In some embodiments, the substrate is made of natural or artificial fibers.

In certain embodiments, the kit comprises a suppository. In some embodiments, the application tool or second application tool comprises a patch. In some embodiments, the application tool or second application tool comprises a sprayer. In some embodiments, the application tool or second application tool comprises a nebulizer. In some embodiments, the application tool or second application tool comprises a capsule configured to release the mixture inside gastrointestinal tract of the subject. In some embodiments, the application tool or second application tool is configured to release the mixture in a controlled manner. In some embodiments, the surface area is selected from the group consisting of: skin, surface areas of oral cavity, nasal cavity, gastrointestinal tract, and respiratory tract, and any combination thereof.

In another aspect, a kit comprises a composition described herein and an alcohol wipe. In another aspect, a kit comprises a composition described herein and a vial containing an alcohol for application to the surface area of a subject.

### Definitions

The present invention will be described with respect to particular embodiments and with reference to certain figures but the invention is not limited thereto but only by the claims. Terms as set forth hereinafter are generally to be understood in their common sense unless indicated otherwise.

The term “polynucleotide” as used herein means a molecule comprising one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

A polyribonucleotide can be present in either linear or circular form. As used herein, the terms “linear RNA” or “linear polyribonucleotide” are used interchangeably, and mean a polyribonucleotide having free 5′ and 3′ ends. In some embodiments, the linear RNA has a free 5′ end or 3′ end. In some embodiments, the linear RNA has non-covalently linked 5′ or 3′ ends. As used herein, the terms “circRNA” or “circular polyribonucleotide” or “circular RNA” are used interchangeably and mean a polyribonucleotide that forms a circular structure through covalent or non-covalent bonds. In some cases, circular polyribonucleotide has a continuous loop in which typical free 5′ and 3′ ends of a corresponding linear polyribonucleotide are joined together via either covalent or non-covalent bond, or via a non-nucleic acid linker (e.g., a non-nucleic acid polymer or a protein). In some cases, circular polyribonucleotide provided herein can be formed by two or more linear polyribonucleotides, which are joined together to form a continuous loop structure, via covalent or non-covalent bonds. While not being bound by theory, it is possible that multiple segments of an RNA can be produced from a DNA and their 5′ and 3′ free ends annealed to produce a “string” of RNA, which ultimately can be circularized when only one 5′ and one 3′ free end remains.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “mixture” means a material made of two or more different substances that are mixed. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, a liquid solution is a liquid-in-liquid solution (e.g., a liquid solute dissolved in a liquid solvent), a solid-in-liquid solution (e.g., a solid solute dissolved in a liquid solvent), or a gas-in-liquid solution (e.g., a solid solute dissolved in a liquid solvent). In some cases, there is more than one solvent and/or more than one solute. In some cases, a mixture is a colloid, liquid suspension, or emulsion. In some cases, a mixture is a solid mixture, e.g., the mixture is present in solid phase.

As used herein, the term “cell-penetrating agent” means an agent that, when contacted to a cell, facilitates entry into the cell. In some cases, a cell-penetrating agent facilitates direct penetration of the cell membrane, for instance, via direct electrostatic interaction with negatively charged phospholipids of the cell membrane, or transient pore formation by inducing configurational changes in membrane proteins or the phospholipid bilayer. In some cases, a cell-penetrating agent facilitates endocytosis-mediated translocation into the cell. For example, under certain situation, the cell-penetrating agent can stimulate the cell to undergo the endocytosis process, by which the cell membrane can fold inward into the cell. In certain embodiments, a cell-penetrating agent helps form a transitory structure that transports across the cell membrane. Without wishing to be bound to a particular theory, a cell-penetrating agent as provided herein can increase the permeability of the cell membrane or increase internalization of a molecule into the cell, as a result of which, delivery into the cell can be more efficient when the cell is contacted with the cell-penetrating agent simultaneously as compared to otherwise identical delivery without the cell-penetrating agent.

As used herein, the term “payload” means any molecule delivered by the polyribonucleotide as disclosed herein. In some cases, a payload is a nucleic acid, a protein, a chemical, a ribonucleoprotein, or any combination thereof. In some cases, a payload is a nucleic acid sequence directly contained within the polyribonucleotide as disclosed herein. In some cases, a payload is attached to or associated with the polyribonucleotide as disclosed herein, for instance via complementary hybridization, or via protein-nucleic acid interactions. In certain cases, the payload is a protein encoded by a nucleic acid sequence contained within, attached to, or associated with the polyribonucleotide. In some cases, the “attachment” means covalent bond or non-covalent interaction between two molecules. In some cases, the “association” when used in the context of the interaction between a payload and a polyribonucleotide means that the payload is indirectly linked to the polyribonucleotide via one or more other molecules in between. In some cases, the attachment or association can be transient. In some cases, a payload is attached to or associated with the polyribonucleotide under one condition but not under another condition, for instance, depending on the ambient pH condition or the presence or absence of a stimulus or a binding partner.

The term “biological effect on a cell” means any effect on the cell that can lead to changes, e.g., morphological or functional, on or in the cell. For instance, a biological effect on a cell can include, but is not limited to, a change in signal transduction inside the cell that effects cellular functions, such as, but not limited to, acceleration or deceleration of cell proliferation, survival, apoptosis, or necrosis of the cell, gene transcription and mRNA translation, and certain differentiated cellular functionalities (e.g., activation of immune cells, excitation or inhibition of neurons, hormone secretion from hormone-secreting cells, or engulfing activity by macrophages), or a change in the efficiency of an exogenous molecule entering into the cell, e.g., increase or decrease in cell permeability relative to the exogenous molecule. A biological effect on a cell can result in amelioration of one or more symptoms of a disease a subject is suffering from, or treatment or eradication of the disease in a subject.

The term “biological effect on a tissue” means any effect on the tissue that can lead to changes, e.g., morphological or functional, on or in the tissue. For instance, a biological effect on a tissue can include, but is not limited to, a change in signal transduction inside the tissue that effects cellular functions or effects tissue function, such as, but not limited to, acceleration or deceleration of cell proliferation in the tissue, tissue survival, apoptosis of cells in the tissue, or necrosis of the tissue, gene transcription and mRNA translation of cells in the tissue, and certain differentiated tissues functionalities or a change in the efficiency of an exogenous molecule entering into the tissue, e.g., increase or decrease in tissue permeability relative to the exogenous molecule. A biological effect on a tissue can result in amelioration of one or more symptoms of a disease a subject is suffering from, or treatment or eradication of the disease in a subject.

The term “alcohol” means any organic compound in which the hydroxyl functional group (—OH) is bound to a carbon. An alcohol as discussed herein can include, but is not limited to, monohydric alcohols, polyhydric alcohols, unsaturated aliphatic alcohols, and alicyclic alcohols. In some cases, an alcohol can refer to ethanol. In some cases, an alcohol can include, but is not limited to, methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

As used herein, the term “surface area” of a subject body means any area of a subject that is or has a potential to be exposed to an exterior environment subject body. A surface area of a subject body, e.g., a mammal body, e.g., a human body, can include skin, surface areas of oral cavity, nasal cavity, ear cavity, gastrointestinal tract, respiratory tract, vaginal, cervical, inter uterine, urinary tract, and eye. In some cases, a surface area of a subject body can often refer to the outer area under which epithelial cells are lined up. Skin, for example, can be one type of surface area as discussed herein and can be composed of epidermis and dermis, the former of which forms the outermost layers of kin and can include organized assembly of epithelial cells among many other types of cells.

As used herein, the term “topical delivery” means delivery of a substance to skin or an epithelial layer accessible though non-invasive means, e.g., the intestinal and other gastrointestinal (GI) epithelia or the vaginal epithelium. Topical delivery of a pharmaceutical composition can have a local pharmacodynamic effect on the subject, e.g., the topically delivered pharmaceutical composition has a pharmacodynamic effect at or proximate to the particular part of the body (e.g. skin) where the pharmaceutical composition is delivered. In some other embodiments, topical delivery of a pharmaceutical composition as discussed herein is used only to refer to the delivery mode (locally to, e.g., a specific surface area), whereas the pharmaceutical composition can have either a local or systemic pharmacodynamic effect. For instance, the pharmaceutical composition can either stay local at or proximate to the administration site, or can enter a circulation system (e.g., blood or lymphoid system) of the subject body, through which the pharmaceutical composition can be transported to remote parts of the body that are typically not reachable by the pharmaceutical composition via routes other than the circulation systems.

As used herein, the term “systemic delivery” or “systemic administration” means a route of administration of pharmaceutical compositions or other substances into the circulatory system (e.g., blood or lymphoid system). The systemic administration can include oral administration, parenteral administration, intranasal administration, sublingual administration, rectal administration, transdermal administration, or any combinations thereof. As used herein, the term “non-systemic delivery” or “non-systemic administration” can refer to any other routes of administration than systemic delivery of pharmaceutical compositions or other substances, e.g., the delivered substances do not enter the circulation systems (e.g., blood and lymphoid system) of the subject body.

As used herein, the term “expression sequence” means a nucleic acid sequence that encodes a product, e.g., a peptide or polypeptide, or a regulatory nucleic acid. An exemplary expression sequence that codes for a peptide or polypeptide can include a plurality of nucleotide triads, each of which can code for an amino acid and is termed as a “codon”.

As used herein, the term “modified ribonucleotide” means a nucleotide with at least one modification to the sugar, the nucleobase, or the internucleoside linkage.

As used herein, the term “substantially resistant” means one that has at least 50%, 55%, 60%, 65%, 70%, 75%, 80%, 85%, 90%, 95%, 96%, 97%, 98% or 99% resistance as compared to a reference.

As used herein, the term “immunogenic” means a potential to induce an immune response to a substance. In some cases, an immune response may be induced when an immune system of an organism or a certain type of immune cells is exposed to an immunogenic substance. The term “non-immunogenic” can refer to a lack of or absence of an immune response above a detectable threshold to a substance. In some cases, no immune response is detected when an immune system of an organism or a certain type of immune cells is exposed to a non-immunogenic substance. In some cases, a non-immunogenic polyribonucleotide provided herein does not induce an immune response above a pre-determined threshold when measured by an immunogenicity assay. For example, when an immunogenicity assay is used to measure antibodies raised against a circular polyribonucleotide, a non-immunogenic polyribonucleotide as provided herein can lead to production of antibodies at a level lower than a predetermined threshold. The predetermined threshold can be, for instance, at most 1.5 times, 2 times, 3 times, 4 times, or 5 times the level of antibodies raised by a control reference.

As used herein, the term “complex” means an association between at least two moieties (e.g., chemical or biochemical) that have an affinity for one another.

“Polypeptide” and “protein” are used interchangeably and mean a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, modified amino acids, and amino acid mimetics).

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the terms “pharmaceutically acceptable” and “therapeutically acceptable” are used interchangeably. A “therapeutically acceptable” component means a component that is not biologically or otherwise undesirable, e.g., the component may be incorporated into a pharmaceutical formulation of the invention and administered to a patient as described herein without causing any significant undesirable biological effects or interacting in a deleterious manner with any of the other components of the formulation in which it is contained. When the term “therapeutically acceptable” is used to refer to an excipient, it implies that the component has met the required standards of toxicological and manufacturing testing or that it is included on the Inactive Ingredient Guide prepared by the U.S. Food and Drug Administration.

As used herein, the term “naked delivery” or “naked RNA” means a formulation of RNA for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier.

As used herein, the term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “sterilizing agent” means any agent that is bacteriostatic, bactericidal, and/or actively kills microorganisms, inactivates microorganisms, or prevents microorganisms from growing. A sterilizing agent that kills microorganisms can be antimicrobial and/or antiseptic. In some embodiments, the sterilizing agent is a liquid, such as an alcohol, iodine, or hydrogen peroxide. In some embodiments, the sterilizing agent, is UV light or a laser light. In some embodiments, the sterilizing agent is heat delivered electrically or through other means (e.g., vapor, contact).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 illustrates an example in which both exemplary linear and circular RNA were delivered topically to the ear skin of mice and their RNA levels in the ear tissue were examined over the 6 hour to 3 days after delivery.

FIGS. 2A and 2B are plots summarizing qPCR results from ear punches of mice 6 hours, 1 day, 3 days, or 12 days after topical delivery of linear or circular RNAs with the aid of Boost (ethanol). FIG. 2A shows results from qPCR assays using primers that for detection of both the linear and circular RNAs. FIG. 2B shows results from qPCR assays using primers for detection of the circular RNA, but not the linear RNA.

FIGS. 3A and 3B are plots summarizing qPCR results from ear punches of mice 6 hours, 1 day, 3 days, or 12 days after topical delivery of linear or circular RNAs with the aid of both Boost (ethanol) and TransIT mRNA agent (Minis Bio, LLC), a cationic, non-liposomal polymer lipid transit agent. FIG. 3A shows results from qPCR assays using primers for detection of both the linear and circular RNAs. FIG. 3B shows results from qPCR assays using primers for detection of the circular RNA, but not the linear RNA.

FIG. 4 illustrates the protein expression from topically administered RNA using DMSO gel (RNA) or DMSO gel alone (vehicle).

FIG. 5 illustrates the protein expression from topically administered RNA formulated with Johnson & Johnson baby lotion (RNA) or Johnson & Johnson baby lotion alone (vehicle).

FIG. 6 illustrates protein expression from topically administered RNA with ethanol (RNA) or ethanol alone (vehicle).

FIG. 7 shows fluorescent images (B/W) from topical administration of circRNA-Cy5 results in RNA delivery to tissue.

FIG. 8 shows quantification of fluorescent images from topical administration of circRNA-Cy5 results in RNA delivery to tissue.

FIG. 9 shows fluorescent images (B/W) from topical administration of mRNA-Cy5 results in RNA delivery to tissue.

FIG. 10 shows quantification of fluorescent images from topical administration of mRNA-Cy5 results in RNA delivery to tissue.

FIG. 11 shows topical administration of mRNA results in RNA delivery to tissue at day 1 and day 4 after administration when the tissue is wiped with an ethanol wipe prior to application.

FIG. 12 shows topical administration of mRNA results in RNA delivery to tissue at day 1 and day 4 after administration when tissue is wiped with an isopropyl alcohol wipe prior to application.

FIG. 13 shows topical administration of circular RNA results in RNA delivery to tissue at day 1 and day 4 after administration when tissue is wiped with an ethanol wipe prior to application.

FIG. 14 shows topical administration of circular RNA mixed with 10% ethanol results in RNA delivery to tissue at day 1 and day 4 after administration.

FIG. 15 shows topical administration of circular RNA mixed with 10% isopropyl alcohol results in RNA delivery to tissue at day 1 and day 4 after administration.

FIG. 16 shows topical administration of circular RNA results in RNA delivery to tissue at day 1 and day 4 after administration when the tissue is wiped with an isopropyl alcohol wipe prior to application.

FIG. 17 shows topical administration of linear mRNA mixed with PBS, PBS and 10% ethanol, or PBS and 10% isopropyl alcohol results in RNA delivery to tissue at day 1 after administration, and topical administration of linear mRNA mixed with PBS and 10% ethanol or PBS and 10% isopropyl alcohol results in RNA delivery to tissue at day 4 after administration.

FIG. 18 shows topical administration of circRNA results in expression of functional protein in tissue when tissue is wiped with an ethanol wipe prior to application.

FIG. 19 shows topical administration of circRNA results in RNA delivery to tissue when circRNA is administered with 10% ethanol.

FIG. 20 shows topical administration of circRNA results in RNA delivery to tissue when circRNA is administered with 10% isopropyl alcohol.

FIG. 21 shows topical administration of mRNA results in RNA delivery to tissue when the skin is wiped with an ethanol wipe before application.

FIG. 22 shows topical administration of mRNA results in RNA delivery to tissue when the mRNA is administered with PBS only.

FIG. 23 shows topical administration of mRNA results in RNA delivery to tissue when the mRNA is administered with 10% isopropyl alcohol

### DETAILED DESCRIPTION

This disclosure relates generally to pharmaceutical compositions, preparations, and delivery of polyribonucleotides and applications thereof. The polyribonucleotides can be linear polyribonucleotides, circular polyribonucleotides (circRNAs), or a combination thereof.

In some aspects, the present disclosure provides compositions and methods for delivering polyribonucleotides to a cell. In some cases, the compositions and methods provided herein deliver polyribonucleotides into a cell ex vivo or in vivo. In certain embodiments, the compositions and methods provided herein are particularly useful for topical delivery of polyribonucleotides into a cell in a subject. In some cases, the compositions and methods provided herein deliver polyribonucleotide for therapeutic applications, such as prevention or treatment of disease(s) in a subject.

The compositions disclosed herein can include a mixture of a polyribonucleotide and an alcohol, such as ethanol. The methods disclosed herein can include delivering a polyribonucleotide in a composition comprising a mixture of the polyribonucleotide and an alcohol, such as ethanol. In some aspects, the present disclosure provides a kit comprising a polyribonucleotide and an alcohol (e.g., ethanol) for delivering the polyribonucleotide into a cell. In some embodiments, the kit comprises a sterilizing agent. In a particular embodiment, the kit comprises a polyribonucleotide and an alcohol wipe (e.g., ethanol wipe, isopropyl wipe).

The compositions disclosed herein can include a mixture of a polyribonucleotide and a cell-penetrating agent. The methods disclosed herein can include delivering a polyribonucleotide in a composition comprising a mixture of the polyribonucleotide and a cell-penetrating agent. In some aspects, the present disclosure provides a kit comprising a polyribonucleotide and a cell-penetrating agent for delivery of the polyribonucleotide into a cell. In some embodiments, the kit comprises a sterilizing agent.

The compositions disclosed herein can be a composition free of any carrier comprising a polyribonucleotide and a diluent. This composition can be used in a method of delivery to an epithelial cell.

In some embodiments, the compositions, therapeutic compositions, or pharmaceutical compositions described herein are directly administered to a surface area (e.g., a topical surface area). In some embodiments, the compositions, therapeutic compositions, or pharmaceutical compositions described herein are applied to a surface area of a subject after application of a sterilizing agent.

The compositions, methods, and kits provided herein can offer a simple and effective solution in which to deliver polyribonucleotides into cells. A polyribonucleotide can be delivered into a cell more efficiently in the presence of the cell-penetrating agent than in the absence of the cell-penetrating agent. In some cases, the cell-penetrating agent described herein can increase the efficiency of delivery of the polynucleotide by at least about 10%, 15%, 20%, 30%, 40%, 50%, 60%, 70%, 80%, 90%, 100%, 120%, 150%, 200%, 250%, 300%, 400%, 500%, 600%, 700%, 800%, 900%, 1000%, 2000%, 5000%, 8000%, 10,000%, 20,000%, or 50,000% as compared to the efficiency of delivery of the polynucleotide in the absence of the cell-penetrating agent.

### Compositions for Polyribonucleotide Delivery

In some aspects, the present disclosure provides compositions or pharmaceutical compositions for delivering a polyribonucleotide. The compositions or pharmaceutical compositions can comprise an alcohol (e.g., ethanol) for delivering the polyribonucleotide to a cell. The compositions or pharmaceutical compositions can comprise a cell-penetrating agent. The cell-penetrating agent is configured to enhance delivery of the polyribonucleotide into a cell. The polyribonucleotide can be present in either linear or circular form.

In some cases, the compositions or pharmaceutical compositions provided herein are suitable for therapeutic applications, e.g., the polyribonucleotide is a therapeutic polyribonucleotide that has therapeutic effects when the composition is administered to a subject. In some aspects, the polyribonucleotide encodes a protein to promote wound healing.

In certain aspects, the present disclosure provides therapeutic compositions and methods of administrating the compositions, therapeutic compositions, or pharmaceutical compositions described herein. In some embodiments, the the compositions, therapeutic compositions, or pharmaceutical compositions described herein are directly administered to a surface area (e.g., a topical surface area). In some embodiments, the compositions, therapeutic compositions, or pharmaceutical compositions described herein are applied to a surface area of a subject after application of a sterilizing agent.

The cell-penetrating agent is configured to enhance delivery of the polyribonucleotide into a cell. In some cases, the compositions provided herein are suitable for therapeutic applications, e.g., the polyribonucleotide is a therapeutic polyribonucleotide that has therapeutic effects when the composition is administered to a subject. In aspects, the present disclosure provides therapeutic compositions and methods of administrating the therapeutic compositions described herein. The polyribonucleotide can be present in either linear or circular form.

The compositions or pharmaceutical compositions as described herein can be used for wound treatment. For example, a method treating a wound can comprise contacting the wound, or the tissue surrounding the wound, to a composition or pharmaceutical composition as described herein. In some embodiments, the polyribonucleotide of the composition or pharmaceutical compositions comprises a sequence encoding a growth factor, such as EGF, PDGF, TGFβ, or VEGF.

### Alcohol

Alcohol as described herein can be used for the delivery of a polyribonucleotide into a cell. An alcohol can be in a mixture with a polyribonucleotide as described herein for delivery of the polyribonucleotide into a cell. The mixture can comprise the alcohol in at least about 0.3% v/v alcohol to about 75% v/v. The alcohol can be ethanol. In some embodiments, the mixture is applied to a surface area of a subject. In some embodiments, the mixture is a pharmaceutical composition.

An alcohol can be any alcohol that comprises one or more hydroxyl function groups. In some cases, the alcohol is, but is not limited to, a monohydric alcohol, a polyhydric alcohol, an unsaturated aliphatic alcohol, or an alicyclic alcohol. The alcohol can include one or more of methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the alcohol is ethanol.

In other cases, the compositions, pharmaceutical compositions, and methods provided herein only include an alcohol and do not have or use any other agent to enhance the delivery of the polyribonucleotide into a cell. In some cases, the alcohol is ethanol and the composition, pharmaceutical composition, and methods do not have or use any other agent to enhance delivery of polyribonucleotide into a cell. In some cases, the alcohol is a cell-penentrating agent. In some cases, the alcohol is not a cell-penetrating agent.

The composition disclosed herein can include a mixture of an alcohol and a polyribonucleotide. In some cases, the polyribonucleotide is present in a pre-mixed mixture with the alcohol. In some cases, the polyribonucleotides is provided separately from the alcohol prior to contact to a cell. In these instances, the polyribonucleotide is contacted with the alcohol when being applied to a cell, and becomes mixed together for delivery of the polyribonucleotide into the cell. Without being bound to a certain theory, the concentration of the alcohol in the mixture can contribute to the efficiency of delivery. Therefore, in some cases, the alcohol is provided at a predetermined concentration in the mixture. In some other cases, when the alcohol and the polyribonucleotide are separate initially but mixed together when being applied for delivery, the alcohol is provided at a sufficient amount relative to the polyribonucleotide that would ensure it reach a minimum predetermined concentration in the mixture.

In some cases, the alcohol constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, the alcohol constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, the alcohol constitutes about 10%, about 20%, about 30%, about 40%, about 50%, about 60%, about 70%, about 80%, about 90%, about 95%, about 98%, or about 100% v/v of the mixture.

In some cases, the alcohol constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% weight per weight (w/w) of the mixture. In some cases, the alcohol constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% w/w of the mixture. In some cases, the cell-penetrating agent constitutes about 10%, about 20%, about 30%, about 40%, about 50%, about 60%, about 70%, about 80%, about 90%, about 95%, or about 98% w/w of the mixture. In some cases, the alcohol constitutes about 10% v/v of the mixture.

In some embodiments, the alcohol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture, or any percentage v/v therebetween. In some embodiments, the alcohol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture, or any percentage v/v therebetween.

In some cases, the mixture described herein is a liquid solution. For instance, the alcohol is a liquid substance itself. In these cases, the polyribonucleotide can also be dissolved in the liquid solution.

In some cases, ethanol constitutes at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, ethanol constitutes at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, ethanol constitutes about 10%, about 20%, about 30%, about 40%, about 50%, about 60%, about 70%, about 80%, about 90%, about 95%, about 98%, or about 100% v/v of the mixture. In some cases, ethanol constitutes about 10% v/v of the mixture.

In some embodiments, the ethanol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture, or any percentage v/v therebetween. In some embodiments, the ethanol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture, or any percentage v/v therebetween.

### Cell-Penetrating Agents

The cell-penetrating agent described herein can include any substance that enhances delivery of a polyribonucleotide into a cell. The cell-penetrating agent can include an organic compound or an inorganic molecule. In some cases, the cell-penetrating agent is an organic compound having one or more functional groups such as, but not limited to, alkane, alkene, and arene; Halogen-substituted alkane, alkenes, and arenes; alcohols, phenols (derivatives of benzene), ethers, aldehydes, ketones, and carboxylic acids; amines and nitriles. In some embodiments, the cell-penetrating agent is soluble in polar solvents. In some embodiments, the cell-penetrating agent is insoluble in polar solvents. The polyribonucleotide can be present in either linear or circular form.

The cell-penetrating agent can include organic compounds such as alcohols having one or more hydroxyl function groups. In some cases, the cell-penetrating agent includes an alcohol such as, but not limited to, monohydric alcohols, polyhydric alcohols, unsaturated aliphatic alcohols, and alicyclic alcohols. The cell-penetrating agent can include one or more of methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the cell-penetrating agent comprises ethanol.

In other cases, the compositions and methods provided herein only include an alcohol as the cell-penetrating agent, and do not have or use any other agent to enhance the delivery of the polyribonucleotide into a cell. In some cases, the cell-penetrating agent comprises ethanol and any other alcohol that can enhance delivery of polyribonucleotide into a cell. In some cases, the cell-penetrating agent comprises ethanol and any other organic or inorganic molecules that can enhance delivery of polyribonucleotide into a cell. In some cases, the cell-penetrating agent comprises ethanol and liposome or nanoparticles such as those described in International Publication Nos. WO2013006825, WO2016036735, WO2018112282A1, and WO2012031043A1, each of which is incorporated herein by reference in its entirety. In some cases, the cell-penetrating agent comprises ethanol and cell-penetrating peptides or proteins such as those described in Bechara et al, Cell-penetrating peptides: 20 years later, where do we stand? FEBS Letters 587(12):1693-1702 (2013); Langel, Cell-Penetrating Peptides: Processes and Applications (CRC Press, Boca Raton Fla., 2002); El-Andaloussi et al., Curr. Pharm. Des. 11(28):3597-611 (2003); Deshayes et al, Cell. Mol. Life Sci. 62(16): 1839-49 (2005), US Patent Publication Nos. US20130129726, US20130137644 and US20130164219, each of which is herein incorporated by reference in its entirety). In some cases, the ratio of ethanol versus other cell-penetrating agent is about 1:0.001, 1:0.002, 1:005, 1:008, 1:0.01, 1:0.02, 1:0.05, 1:0.08, 1: 0.1, 1:0.2, 1:0.3, 1:0.4, 1:0.5, 1:0.6, 1:0.7, 1:0.8, 1:0.9, 1:1, 1:1.2, 1:1.5, 1:1.8, 1:2, 1:2.5, 1:3, 1:3.5, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:150, 1:200, 1:250, 1:500, or 1:1000. In some cases, the ratio of ethanol versus other cell-penetrating agent is at least about 1:0.001, 1:0.002, 1:005, 1:008, 1:0.01, 1:0.02, 1:0.05, 1:0.08, 1:0.1, 1:0.2, 1:0.3, 1:0.4, 1:0.5, 1:0.6, 1:0.7, 1:0.8, 1:0.9, 1:1, 1:1.2, 1:1.5, 1:1.8, 1:2, 1:2.5, 1:3, 1:3.5, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:150, 1:200, 1:250, or 1:500.

The composition disclosed herein can include a mixture of a cell-penetrating agent and a polyribonucleotide. In some cases, the polyribonucleotide is present in a pre-mixed mixture with the cell-penetrating agent. In some cases, the polyribonucleotides is provided separately from the cell-penetrating agent prior to contact to a cell. In these instances, the polyribonucleotide is contacted with the cell-penetrating agent when being applied to a cell, and becomes mixed together for delivery of the polyribonucleotide into the cell. Without being bound to a certain theory, the concentration of the cell-penetrating agent in the mixture can contribute to the efficiency of delivery. Therefore, in some cases, the cell-penetrating agent is provided at a predetermined concentration in the mixture. In some other cases, when the cell-penetrating agent and the polyribonucleotide are separate initially but mixed together when being applied for delivery, the cell-penetrating agent is provided at a sufficient amount relative to the polyribonucleotide that would ensure it reach a minimum predetermined concentration in the mixture.

In some cases, the cell-penetrating agent constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, the cell-penetrating agent constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, the cell-penetrating agent constitutes about 10%, about 20%, about 30%, about 40%, about 50%, about 60%, about 70%, about 80%, about 90%, about 95%, about 98%, or about 100% v/v of the mixture.

In some cases, the cell-penetrating agent constitutes at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% weight per weight (w/w) of the mixture. In some cases, the cell-penetrating agent constitutes at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% w/w of the mixture. In some cases, the cell-penetrating agent constitutes about 10%, about 20%, about 30%, about 40%, about 50%, about 60%, about 70%, about 80%, about 90%, about 95%, or about 98% w/w of the mixture. In some cases, the cell-penetrating agent constitutes about 10% v/v of the mixture.

In some cases, the mixture described herein is a liquid solution. For instance, the cell-penetrating agent is a liquid substance itself. Alternatively, the cell-penetrating agent is a solid, liquid, or gas substance and dissolved in a liquid carrier, e.g., water. In these cases, the polyribonucleotide can also be dissolved in the liquid solution.

In some cases, ethanol constitutes at least about 0.1%, at least about 0.2%, at least about 0.3%, at least about 0.4%, at least about 0.5%, at least about 0.6%, at least about 0.7%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 98% volume per volume (v/v) of the mixture. In some cases, ethanol constitutes at most about 0.1%, 0.2%, 0.3%, 0.4%, 0.5%, 0.6%, 0.7%, 0.9%, 1%, 2%, 3%, 4%, 5%, 6%, 7%, 8%, 9%, 10%, 20%, 30%, 40%, 50%, 60%, 70%, 80%, or 90% v/v of the mixture. In some cases, ethanol constitutes about 10%, about 20%, about 30%, about 40%, about 50%, about 60%, about 70%, about 80%, about 90%, about 95%, about 98%, or about 100% v/v of the mixture. In some cases, ethanol constitutes about 10% v/v of the mixture.

### Polyribonucleotides

Aspects of the present disclosure relate to compositions and methods for delivering polyribonucleotides into a cell, either ex vivo or in vivo. The polyribonucleotides can be either a linear polyribonucleotide or circular polyribonucleotide. In some cases, the polyribonucleotides have biological effects on the cell or the subject that the polyribonucleotides are administered to. Aspects of the present disclosure provide pharmaceutical compositions comprising polyribonucleotides that have therapeutical effects on a subject, when the composition is delivered into a cell in the subject, e.g., direct administration, or into a cell that is to be administered to the subject. e.g., cell transplantation or cell infusion. The polyribonucleotides as described herein can be mixed with an alcohol (e.g., ethanol) in a pharmaceutical composition. The polyribonucleotides as described herein can be mixed with a diluent in composition that is free of any carrier.

The polyribonucleotide can include sequences for expression products. Alternatively or additionally, the polyribonucleotide includes sequences for binding to other entities (e.g., targets), such as nucleic acids (e.g., RNAs, DNAs, RNA-DNA hybrids), small molecules (e.g., drugs), aptamers, polypeptides, proteins, lipids, phospholipids (e.g. PI(4,5)P2), carbohydrates, antibodies, viruses, virus particles, membranes, multi-component complexes, cells, other cellular moieties, any fragments thereof, and any combination thereof. Expression of sequences from the polyribonucleotide and/or binding of the polyribonucleotide to a target can have various biological effects. In some cases, the polyribonucleotide modulates a cellular function, e.g., transiently or in a long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

The polyribonucleotides can be at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, or at least about 1,000 nucleotides. In some cases, the polyribonucleotide is of a sufficient size to accommodate a binding site for a ribosome for expression of sequences from the polyribonucleotide. In some cases, the polyribonucleotide is of a sufficient size to accommodate a binding site for a target for exhibiting regulatory functions of the polyribonucleotide, such as inhibition of translation of a target mRNA, degradation of a target mRNA, modulation of splicing of a target RNA, and facilitation of binding between a target receptor and its ligand. One of skill in the art can appreciate that the maximum size of a linear or circular polyribonucleotide can be as large as is within the technical constraints of producing a linear or circular polyribonucleotide, and/or using the circular polyribonucleotide. In some cases, the maximum size of a linear or circular polyribonucleotide provided herein can be limited by the ability of packaging and delivering the RNA to a target. In some cases, the size of a polyribonucleotide is a length sufficient to encode useful polypeptides, and thus, lengths of less than about 20,000 nucleotides, less than about 15,000 nucleotides, less than about 10,000 nucleotides, less than about 7,500 nucleotides, or less than about 5,000 nucleotides, less than about 4,000 nucleotides, less than about 3,000 nucleotides, less than about 2,000 nucleotides, less than about 1,000 nucleotides, less than about 500 nucleotides, less than about 400 nucleotides, less than about 300 nucleotides, less than about 200 nucleotides, less than about 100 nucleotides may be useful.

The polyribonucleotide provided herein can have one or more modifications, such as substitutions, insertions and/or additions, deletions, and covalent modifications with respect to reference sequences, in particular, the parent polyribonucleotide. For example, the polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The polyribonucleotide can include at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some cases, the mRNA includes at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some cases, the mRNA includes at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some cases, mRNA includes at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In some embodiments, the polyribonucleotide lacks a cap. In some embodiments, the polyribonucleotide lacks a poly-A tail. In some embodiments, the polyribonucleotide is non-immunogenic. In some embodiments, the polyribonucleotide is immunogenic.

The polyribonucleotide can include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Different sugar modifications, nucleotide modifications, and/or internucleoside linkages (e.g., backbone structures) can exist at various positions in a polyribonucleotide provided herein. One of ordinary skill in the art will appreciate that the nucleotide analogs or other modification(s) can be located at any position(s) of the polyribonucleotide, such that the function of the polyribonucleotide is not substantially decreased. The polyribonucleotide can include from about 1% to about 100% modified nucleotides (either in relation to overall nucleotide content, or in relation to one or more types of nucleotide, e.g., any one or more of A, G, U, or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some cases, a concentration of the polyribonucleotide in the mixture is at least about 0.1 ng/mL, at least about 0.2 ng/mL, at least about 0.5 ng/mL, at least about 1 ng/mL, at least about 5 ng/mL, at least about 10 ng/mL, at least about 20 ng/mL, at least about 50 ng/mL, at least about 100 ng/mL, at least about 200 ng/mL, at least about 500 ng/mL, at least about 1 μg/mL, at least about 2 μg/mL, at least about 3 μg/mL, at least about 4 μg/mL, at least about 5 μg/mL, at least about 10 μg/mL, at least about 20 μg/mL, at least about 30 μg/mL, at least about 40 μg/mL, at least about 50 μg/mL, at least about 100 μg/mL, at least about 200 μg/mL, at least about 300 μg/mL, at least about 400 μg/mL, at least about 500 μg/mL, at least about 1 mg/mL, at least about 2 mg/mL, at least about 5 mg/mL, at least about 10 mg/mL, at least about 20 mg/mL, at least about 50 mg/mL, or at least about 100 mg/mL. In some cases, a concentration of the polyribonucleotide in the mixture is at most about 0.1 ng/mL, at most about 0.2 ng/mL, at most about 0.5 ng/mL, at most about 1 ng/mL, at most about 5 ng/mL, at most about 10 ng/mL, at most about 20 ng/mL, at most about 50 ng/mL, at most about 100 ng/mL, at most about 200 ng/mL, at most about 500 ng/mL, at most about 1 μg/mL, at most about 2 μg/mL, at most about 3 μg/mL, at most about 4 μg/mL, at most about 5 μg/mL, at most about 10 μg/mL, at most about 20 μg/mL, at most about 30 μg/mL, at most about 40 μg/mL, at most about 50 μg/mL, at most about 100 μg/mL, at most about 200 μg/mL, at most about 300 μg/mL, at most about 400 μg/mL, at most about 500 μg/mL, at most about 1 mg/mL, at most about 2 mg/mL, at most about 5 mg/mL, at most about 10 mg/mL, at most about 20 mg/mL, at most about 50 mg/mL, or at most about 100 mg/mL.

In some cases, a concentration of the polyribonucleotide in the mixture is about 0.1 ng/mL, about 0.2 ng/mL, about 0.5 ng/mL, about 1 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, about 3 μg/mL, about 4 μg/mL, about 5 μg/mL, about 10 μg/mL, about 20 μg/mL, about 30 μg/mL, about 40 μg/mL, about 50 μg/mL, about 100 μg/mL, about 200 μg/mL, about 300 μg/mL, about 400 μg/mL, about 500 μg/mL, about 1 mg/mL, about 2 mg/mL, about 5 mg/mL, about 10 mg/mL, about 20 mg/mL, about 50 mg/mL, or about 100 mg/mL.

The composition can comprise a polyribonucleotide and an alcohol (e.g., ethanol). In certain particular embodiments, the composition comprises a linear polyribonucleotide and an alcohol. In certain particular embodiments, the composition comprises a circular polyribonucleotide (circRNA) and an alcohol. Due to the circular structure, circRNA have improved stability, increased half-life, reduced immunogenicity, and/or improved functionality (e.g., of a function described herein) compared to a corresponding linear RNA.

The composition can comprise a polyribonucleotide and a cell-penetrating agent. In certain particular embodiments, the composition comprises a linear polyribonucleotide and a cell-penetrating agent. In certain particular embodiments, the composition comprises a circular polyribonucleotide (circRNA) and a cell-penetrating agent. Due to the circular structure, circRNA have improved stability, increased half-life, reduced immunogenicity, and/or improved functionality (e.g., of a function described herein) compared to a corresponding linear RNA.

In some cases, the circular polyribonucleotide provided herein has a half-life of at least that of a linear counterpart, e.g., linear expression sequence, or linear circular polyribonucleotide. In some cases, the circular polyribonucleotide has a half-life that is increased over that of a linear counterpart. In some cases, the half-life is increased by about 5%, 10%, 15%, 20%, 25%, 30%, 35%, 40%, 45%, 50%, or greater. In some cases, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

### Payload and Biological Effects

In some cases, the polyribonucleotide comprises at least one payload that has a biological effect on the cell or the subject upon or after delivery to the cell or subject. The payload can be one or more sequences encoded in the polyribonucleotide that are expressed, bind to a target entity, such as a target molecule (e.g., protein, nucleic acids, small molecules, or robzymes), or a target cell. The polyribonucleotide can be present in either linear or circular form. The biological effect on a cell can comprise a variety of molecular and cellular changes on the cell that lead to changes, e.g., morphological or functional, on or in the cell. For instance, a biological effect on a cell can include, but is not limited to, a change in signal transduction inside the cell that effects cellular function, such as, but not limited to, acceleration or deceleration of cell proliferation, survival, apoptosis, or necrosis of the cell, gene transcription and mRNA translation, and certain differentiated cellular functionalities (e.g., activation of immune cells, excitation or inhibition of neurons, hormone secretion from hormone-secreting cells, or engulfing activity by macrophages), or a change in the efficiency of an exogenous molecule entering into the cell, e.g., increase or decrease in cell permeability relative to the exogenous molecule. The biological effect on a subject can also include a variety of changes to the subject's physiology, e.g., structural or functional changes to any one or more tissues or organs. The biological effect on a subject can lead to changes in one or more physiological parameters that can be measured from the subject, such as ECG, blood glucose, blood pressure, body temperature, blood count, HbCO, and MetHb. The biological effects can also include amelioration of one or more symptoms of a disease the subject is suffering from, or treatment or eradication of the disease in the subject. The biological effect can also include wound healing.

### Expression Sequence

In some cases, the polyribonucleotide as described herein comprises at least one expression sequence that encodes a peptide or polypeptide. The peptide may include, but is not limited to, a protein, a small peptide, a peptidomimetic (e.g., peptoid), amino acids, and amino acid analogs. The peptide may be linear or branched. The peptide may have a molecular weight less than about 500,000 grams per mole, a molecular weight less than about 200,000 grams per mole, a molecular weight less than about 100,000 grams per mole, a molecular weight less than about 50,000 grams per mole, a molecular weight less than about 20,000 grams per mole, a molecular weight less than about 10,000 grams per mole, a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. The peptide can comprise, for example, a neurotransmitter, a hormone, a drug, a toxin, a viral or microbial particle, a synthetic molecule, and an agonist or antagonist thereof. The polyribonucleotide can be present in either linear or circular form.

Some examples of a peptide or polypeptide expressed by the polyribonucleotide provided herein include a fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. Peptides described herein can also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, an intra-organellar antigen.

In some cases, the polyribonucleotide comprises an expression sequence encoding a protein, e.g., a therapeutic protein. Some examples of therapeutic proteins include, but are not limited to, a protein replacement, protein supplementation, vaccination, antigens (e.g., tumor antigens, viral, bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof. In some embodiments, the protein or therapeutic protein is a wound healing protein, such as a growth factor. For example, the growth factor is EGF, PDGF, TGFβ, or VEGF.

In some embodiments, the protein or therapeutic protein is used in a method of wound healing. For example, a method treating a wound can comprise contacting the wound, or the tissue surrounding the wound, to a composition or pharmaceutical composition as described herein, wherein the polyribonucleotide of the composition or pharmaceutical composition comprises a sequence encoding a growth factor, such as EGF, PDGF, TGFβ, or VEGF.

In some cases, the polyribonucleotide comprises a regulatory element, e.g., a sequence that modifies expression of an expression sequence within a circular polyribonucleotide or a linear polyribonucleotide. A regulatory element can encode a sequence that is located adjacent to an expression sequence that encodes an expression product. A regulatory element can be linked operatively to the adjacent sequence. A regulatory element can increase an amount of product expressed as compared to an amount of the expressed product without the presence of the regulatory element. In addition, one regulatory element can increase an amount of products expressed for multiple expression sequences attached in tandem. Hence, one regulatory element can enhance the expression of one or more expression sequences. Multiple regulatory elements are well-known to persons of ordinary skill in the art.

In some cases, the polyribonucleotide comprises a translation initiation sequence, e.g., a start codon. In some cases, the translation initiation sequence is a Kozak or Shine-Dalgarno sequence.

In some cases, the polyribonucleotide initiates at a codon which is not the first start codon, e.g., AUG. Translation of the polyribonucleotide can initiate at an alternative translation initiation sequence, such as, but not limited to, ACG, AGG, AAG, CTG/CUG, GTG/GUG, ATA/AUA, ATT/AUU, TTG/UUG. In some cases, translation is initiated by eukaryotic initiation factor 4A (eIF4A). In other embodiments, translation is initiated from an internal ribosome entry site (IRES) element of the polyribonucleotide. An IRES element can comprise an RNA sequence capable of engaging a eukaryotic ribosome. In some cases, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some cases, the polyribonucleotide comprises one or more expression sequences and each expression sequence can or can not have a termination element. In some cases, the polyribonucleotide comprises one or more expression sequences and the expression sequences lack a termination element. In some embodiments, the polyribonucleotide is a circular polyribonucleotide that lacks a termination element such that the circular polyribonucleotide is continuously translated. In some cases, the polyribonucleotide includes a termination element at the end of one or more expression sequences. In some cases, one or more expression sequences lacks a termination element. Generally, termination elements comprise an in-frame nucleotide triplet that signals termination of translation, e.g., UAA, UGA, UAG.

The polyribonucleotide can comprise a regulatory nucleic acid, e.g., that modifies expression of an endogenous gene and/or an exogenous gene. In some cases, the polyribonucleotide comprises one or more expression sequences that encode (e.g., are complementary to) a regulatory nucleic acid. A regulatory nucleic acid can include, but is not limited to, a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

In one example, the regulatory nucleic acid targets a host gene. The regulatory nucleic acid can include, but is not limited to, a nucleic acid that hybridizes to an endogenous gene (e.g., miRNA, siRNA, mRNA, lncRNA, RNA, DNA, an antisense RNA, gRNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, and a nucleic acid that modulates a DNA or RNA binding factor. In one embodiment, the sequence is an miRNA.

In some cases, the polyribonucleotide comprises a regulatory nucleic acid, such as a guide RNA (gRNA). In some cases, the polyribonucleotide comprises a guide RNA or encodes the guide RNA. A gRNA can be a short synthetic RNA composed of a “scaffold” sequence necessary for binding to a user-defined ˜20 nucleotide targeting sequence for a genomic target. The gRNA can recognize specific DNA sequences (e.g., sequences adjacent to or within a promoter, enhancer, silencer, or repressor of a gene). The gRNA can be part of a CRISPR system for gene editing. For the purposes of gene editing, the polyribonucleotide can be designed to include one or multiple guide RNA sequences corresponding to a desired target DNA sequence; see, for example, Cong et al. (2013) Science, 339:819-823; Ran et al. (2013) Nature Protocols, 8:2281-2308, each of which is incorporated by reference herein in its entirety.

The polyribonucleotide can encode a regulatory nucleic acid substantially complementary, or fully complementary, to all or a fragment of an endogenous gene or gene product (e.g., mRNA). The regulatory nucleic acid can be complementary to sequences at the boundary between introns and exons, in between exons, or adjacent to exon, to prevent the maturation of newly-generated nuclear RNA transcripts of specific genes into mRNA for transcription. A regulatory nucleic acid that is complementary to a specific gene can hybridize with the mRNA for that gene and prevent its translation. The antisense regulatory nucleic acid can be DNA, RNA, or a derivative or hybrid thereof. In some cases, the regulatory nucleic acid comprises a protein-binding site that binds to a protein that participates in regulation of expression of an endogenous gene or an exogenous gene.

In some cases, the translation efficiency of a circular polyribonucleotide as provided herein is greater than a reference, e.g., a linear counterpart, a linear expression sequence, or a linear circular polyribonucleotide. In some cases, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some cases, a circular polyribonucleotide has a translation efficiency 10% greater than that of a linear counterpart. In some cases, a circular polyribonucleotide has a translation efficiency 300% greater than that of a linear counterpart.

In some cases, the circular polyribonucleotide produces stoichiometric ratios of expression products. Rolling circle translation continuously produces expression products at substantially equivalent ratios. In some cases, the circular polyribonucleotide has a stoichiometric translation efficiency, such that expression products are produced at substantially equivalent ratios. In some cases, the circular polyribonucleotide has a stoichiometric translation efficiency of multiple expression products, e.g., products from 2, 3, 4, 5, 6, 7, 8, 9, 10, 11, 12, or more expression sequences.

In some cases, once translation of the circular polyribonucleotide is initiated, the ribosome bound to the circular polyribonucleotide does not disengage from the circular polyribonucleotide before finishing at least one round of translation of the circular polyribonucleotide. In some cases, the circular polyribonucleotide as described herein is competent for rolling circle translation. In some cases, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10⁵ rounds, or at least 10⁶ rounds of translation of the circular polyribonucleotide.

In some cases, the rolling circle translation of the circular polyribonucleotide leads to generation of polypeptide product that is translated from more than one round of translation of the circular polyribonucleotide (“continuous” expression product). In some cases, the circular polyribonucleotide comprises a stagger element (e.g., an element that causes a ribosomal pause during translation), and rolling circle translation of the circular polyribonucleotide leads to generation of polypeptide product that is generated from a single round of translation or less than a single round of translation of the circular polyribonucleotide (allows for production of “discrete” expression products). In some cases, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some cases, the amount ratio of the discrete products over the total polypeptides is tested in an in vitro translation system. In some cases, the in vitro translation system used for the test of amount ratio comprises rabbit reticulocyte lysate. In some cases, the amount ratio is tested in an in vivo translation system, such as a eukaryotic cell or a prokaryotic cell, a cultured cell or a cell in an organism.

In some cases, the polyribonucleotide comprises untranslated regions (UTRs). UTRs of a genomic region comprising a gene may be transcribed but not translated. In some cases, a UTR may be upstream of a translation initiation sequence of an expression sequence described herein. In some cases, a UTR may be included downstream of an expression sequence described herein. In some instances, one UTR for first expression sequence is the same as, continuous with, or overlapping with another UTR for a second expression sequence. In some cases, the intron is a human intron. In some cases, the intron is a full length human intron, e.g., ZKSCAN1.

In some cases, the polyribonucleotide comprises a UTR with one or more stretches of Adenosines and Uridines embedded within. These AU rich signatures may increase turnover rates of the expression product.

Introduction, removal, or modification of UTR AU rich elements (AREs) may be useful to modulate the stability or immunogenicity of a polyribonucleotide. When engineering specific polyribonucleotides, one or more copies of an ARE may be introduced to the polyribonucleotide and the copies of an ARE may modulate translation and/or production of an expression product. Likewise, AREs may be identified and removed or engineered into the polyribonucleotide to modulate the intracellular stability and thus affect translation and production of the resultant protein.

It should be understood that any UTR from any gene may be incorporated into the respective flanking regions of the polyribonucleotide. As a non-limiting example, the UTR or a fragment thereof which may be incorporated is a UTR listed in US Provisional Application Nos. U.S. 61/775,509 and U.S. 61/829,372, or in International Patent Application No. PCT/US2014/021522; the contents of each of which are herein incorporated by reference in its entirety. Furthermore, multiple wild-type UTRs of any known gene may be utilized. It is also within the scope of the present invention to provide artificial UTRs which are not variants of wild type genes. These UTRs or portions thereof may be placed in the same orientation as in the transcript from which they were selected or may be altered in orientation or location. Hence a 5′ or 3′ UTR may be inverted, shortened, lengthened, made chimeric with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, a double, triple, or quadruple UTR, such as a 5′ or 3′ UTR, may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

In some cases, the polyribonucleotide may include a poly-A sequence. In some cases, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some cases, the poly-A sequence is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A sequence is designed relative to the length of the overall polyribonucleotide. This design may be based on the length of the coding region, the length of a particular feature or region (such as the first or flanking regions), or based on the length of the ultimate product expressed from the polyribonucleotide. In this context, the poly-A sequence may be 10, 20, 30, 40, 50, 60, 70, 80, 90, or 100% greater in length than the circular polyribonucleotide or a feature thereof. The poly-A sequence may also be designed as a fraction of polyribonucleotide to which it belongs. In this context, the poly-A sequence may be 10, 20, 30, 40, 50, 60, 70, 80, or 90% or more of the total length of the construct or the total length of the construct minus the poly-A sequence. Further, engineered binding sites and conjugation of circular polyribonucleotide for Poly-A binding protein may enhance expression.

In one embodiment, the polyribonucleotide is designed to include a polyA-G quartet. The G-quartet can be a cyclic hydrogen bonded array of four guanine nucleotides that can be formed by G-rich sequences in both DNA and RNA. In one embodiment, the G-quartet is incorporated at the end of the poly-A sequence. In some cases, the polyA-G quartet results in protein production equivalent to at least 75% of that seen using a poly-A sequence of 120 nucleotides alone.

In some cases, the polyribonucleotide comprises a polyA, lacks a polyA, or has a modified polyA to modulate one or more characteristics of the polyribonucleotide. In some cases, the polyribonucleotide lacking a polyA or having modified polyA improves one or more functional characteristics, e.g., immunogenicity, half-life, expression efficiency, etc.

### Scaffold

In some cases, a polyribonucleotide provided herein binds one or more targets. In one embodiment, the polyribonucleotide binds both a DNA target and a protein target and, e.g., mediates transcription. In another embodiment, the polyribonucleotide brings together a protein complex and, e.g., mediates signal transduction. In another embodiment, the polyribonucleotide binds two or more different targets, such as proteins, and e.g., shuttles these proteins to the cytoplasm. The polyribonucleotide can be present in either linear or circular form.

In some embodiments, a polyribonucleotide binds at least one of DNA, RNA, and proteins and thereby regulates cellular processes (e.g., alters protein expression). In some embodiments, synthetic polyribonucleotide comprises binding sites for interaction with at least one moiety, e.g., a binding moiety, of DNA, RNA, or proteins of choice to thereby compete in binding with the endogenous counterpart.

In one embodiment, a synthetic polyribonucleotide binds and/or sequesters miRNAs. In another embodiment, synthetic polyribonucleotide binds and/or sequesters proteins. In another embodiment, synthetic polyribonucleotide binds and/or sequesters mRNA. In another embodiment, synthetic polyribonucleotide binds and/or sequesters ribosomes. In another embodiment, synthetic polyribonucleotide binds and/or sequesters polyribonucleotide. In another embodiment, synthetic polyribonucleotide binds and/or sequesters long-noncoding RNA (lncRNA) or any other non-coding RNA, e.g., miRNA, tRNA, rRNA, snoRNA, ncRNA, siRNA, long-noncoding RNA, shRNA. Besides binding and/or sequestration sites, the polyribonucleotide may include a degradation element, which results in degradation of the bound and/or sequestered RNA and/or protein.

In one embodiment, a polyribonucleotide comprises a lncRNA or a sequence of a lncRNA, e.g., a polyribonucleotide comprises a sequence of a naturally occurring, non-circular lncRNA or a fragment thereof. In one embodiment, a lncRNA or a sequence of a lncRNA is circularized, with or without a spacer sequence, to form a synthetic circular polyribonucleotide.

In one embodiment, a polyribonucleotide has ribozyme activity. In one embodiment, a polyribonucleotide acts as a ribozyme and cleaves pathogenic or endogenous RNA, DNA, small molecules or protein. In one embodiment, a polyribonucleotide has enzymatic activity. In one embodiment, synthetic polyribonucleotide specifically recognizes and cleaves RNA (e.g., viral RNA). In another embodiment, a polyribonucleotide specifically recognizes and cleaves proteins. In another embodiment, polyribonucleotide specifically recognizes and degrades small molecules.

In one embodiment, a polyribonucleotide is an immolating, self-cleaving, or cleavable polyribonucleotide. In one embodiment, a polyribonucleotide can be used to deliver RNA, e.g., miRNA, tRNA, rRNA, snoRNA, ncRNA, siRNA, long-noncoding RNA, shRNA. In one embodiment, synthetic polyribonucleotide is made up of microRNAs separated by (1) self-cleavable elements (e.g., hammerhead, splicing element), (2) cleavage recruitment sites (e.g., ADAR), (3) a degradable linker (glycerol), (4) a chemical linker, and/or (5) a spacer sequence. In another embodiment, a synthetic polyribonucleotide is made up of siRNAs separated by (1) self-cleavable elements (e.g., hammerhead, splicing element), (2) cleavage recruitment sites (e.g., ADAR), (3) a degradable linker (glycerol), (4), chemical linker, and/or (5) a spacer sequence.

In one embodiment, a polyribonucleotide is a transcriptionally/replication competent polyribonucleotide. This polyribonucleotide can encode any type of RNA. In one embodiment, a synthetic polyribonucleotide comprises an anti-sense miRNA and a transcriptional element. In one embodiment, after transcription, linear functional miRNAs are generated from a circular polyribonucleotide.

In one embodiment, the polyribonucleotide comprises one or more of the above attributes in combination with a translating element.

The polyribonucleotide can comprise at least one binding site for a binding moiety of a target. Targets include, but are not limited to, nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, other cellular moieties, any fragments thereof, and any combination thereof (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, a target is a polypeptide, a protein, or any fragment thereof. For example, a target is a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a phosphatase, an aromatase, a helicase, a protease, an oxidoreductase, a reductase, a transferase, a hydrolase, a lyase, an isomerase, a glycosylase, a extracellular matrix protein, a ligase, an ion transporter, a channel, a pore, an apoptotic protein, a cell adhesion protein, a pathogenic protein, an aberrantly expressed protein, an transcription factor, a transcription regulator, a translation protein, a chaperone, a secreted protein, a ligand, a hormone, a cytokine, a chemokine, a nuclear protein, a receptor, a transmembrane receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, mammalian, or insect cells (e.g., proteins overexpressed by the organisms). In some embodiments, a target is a polypeptide with a mutation, insertion, deletion, or polymorphism. In some embodiments, a target is an antigen, such as a polypeptide used to immunize an organism or to generate an immune response in an organism, such as for antibody production.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can comprise a heavy chain variable region (V_{H}) and a heavy chain constant region. The heavy chain constant region can comprise three domains, C_{H1}, C_{H2} and C_{H3}. Each light chain can comprise a light chain variable region (V_{L}) and a light chain constant region. The light chain constant region can comprise of one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can comprise three CDRs and four FRs arranged from amino-terminus to carboxy-terminus in the following order: FR₁, CDR₁, FR₂, CDR₂, FR₃, CDR₃, and FR₄. The constant regions of the antibodies may mediate the binding of the immunoglobulin to host tissues or factors, including various cells of the immune system (e.g., effector cells) and the first component (C1q) of the classical complement system. The antibodies can be of any isotype (e.g., IgG, IgE, IgM, IgD, IgA and IgY), class (e.g., IgG₁, IgG₂, IgG₃, IgG₄, IgA₁ and IgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} can be encoded by separate genes, they can be joined, using recombinant methods, by an artificial peptide linker that enables them to be made as a single protein chain. Such single chain antibodies include one or more antigen binding moieties. These antibody fragments can be obtained using conventional techniques known to those of skill in the art, and the fragments can be screened for utility in the same manner as are intact antibodies. Antibodies can be human, humanized, chimeric, isolated, dog, cat, donkey, or sheep, or any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is entirely fortuitous and plays no role whatsoever in the binding of an aptamer to its cognate target. Aptamers must also be differentiated from the naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand are short, isolated, non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences can differ from aptamers as a result of the relatively low binding affinity used in nature as compared to tightly binding aptamers. Aptamers are capable of specifically binding to selected partners and modulating the partner's activity or binding interactions, e.g., through binding, aptamers may block their partner's ability to function. The functional property of specific binding to a partner is an inherent property an aptamer. A typical aptamer is 6-35 kDa in size (20-100 nucleotides), binds its partner with micromolar to sub-nanomolar affinity, and may discriminate against closely related targets (e.g., aptamers may selectively bind related proteins from the same gene family). Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides.

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a target is a cell. For example, a target is an intact cell, a cell treated with a compound (e.g., a drug), a fixed cell, a lysed cell, or any combination thereof. In some embodiments, a target is a single cell. In some embodiments, a target is a plurality of cells.

In some embodiments, a single target or a plurality of (e.g., two or more) targets have a plurality of binding moieties. In one embodiment, the single target may have 2, 3, 4, 5, 6, 7, 8, 9, 10, or more binding moieties. In one embodiment, two or more targets are in a sample, such as a mixture or library of targets, and the sample comprises two or more binding moieties. In some embodiments, a single target or a plurality of targets comprise a plurality of different binding moieties. For example, a plurality may include at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties.

A target can comprise a plurality of binding moieties comprising at least 2 different binding moieties. For example, a binding moiety can comprise a plurality of binding moieties comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different binding moieties.

In some instances, a polyribonucleotide comprises one binding site. In some instances, the polynucleotide comprises at least two binding sites. For example, a polyribonucleotide can comprise 2, 3, 4, 5, 6, 7, 8, 9, 10, 11, 12, 13, 14, 15, 16, 17, 18, 19, 20, or more binding sites. In some embodiments, a polyribonucleotide described herein is a molecular scaffold that binds one or more binding moieties of one or more targets. Each target may be, but is not limited to, a different or the same nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, cellular moieties, any fragments thereof, and any combination thereof. In some embodiments, the one or more binding sites bind to one or more binding moieties of the same target. In some embodiments, the one or more binding sites bind to one or more binding moieties of different targets. In some embodiments, circRNA act as scaffolds for one or more binding moieties of one or more targets. In some embodiments, a polyribonucleotide modulates cellular processes by specifically binding to one or more binding moieties of one or more targets. In some embodiments, a polyribonucleotide described herein comprises binding sites for one or more specific targets of interest. In some embodiments, polyribonuclotide comprises multiple binding sites or a combination of binding sites for each binding moiety of interest. For example, a polyribonucleotide comprises a binding site for a polynucleotide target, such as a DNA or RNA. For example, a polyribonucleotide comprises a binding site for an mRNA target. For example, a polyribonucleotide comprises a binding site for an rRNA target. For example, a polyribonucleotide comprises a binding site for a tRNA target. For example, a polyribonucleotide comprises a binding site for genomic DNA target.

In some instances, a polyribonucleotide comprises a binding site for a binding moiety on a single-stranded DNA. In some instances, a polynucleotide comprises a binding site for a binding moiety on a double-stranded DNA. In some instances, a polyribonucleotide comprises a binding site for a binding moiety on an antibody. In some instances, a polyribonucleotide comprises a binding site for a binding moiety on a virus particle. In some instances, a polyribonucleotide comprises a binding site for a binding moiety on a small molecule. In some instances, a polyribonucleotide comprises a binding site for a binding moiety in or on a cell. In some instances, a polyribonucleotide comprises a binding site for a binding moiety on a RNA-DNA hybrid. In some instances, a polyribonucleotide comprises a binding site for a binding moiety on a methylated polynucleotide. In some instances, a polyribonucleotide comprises a binding site for a binding moiety on an unmethylated polynucleotide. In some instances, a polyribonucleotide comprises a binding site for a binding moiety on an aptamer. In some instances, a polyribonucleotide comprises a binding site for a binding moiety on a polypeptide. In some instances, a polyribonucleotide comprises a binding site for a binding moiety on a polypeptide, a protein, a protein fragment, a tagged protein, an antibody, an antibody fragment, a small molecule, a virus particle (e.g., a virus particle comprising a transmembrane protein), or a cell.

In some instances, a binding moiety comprises at least two amide bonds. In some instances, a binding moiety does not comprise a phosphodiester linkage. In some instances, a binding moiety is not DNA or RNA.

A polyribonucleotide provided herein can comprise one or more binding sites for binding moieties on a complex. The polyribonucleotide provided herein can include one or more binding sites for targets to form a complex. The polyribonucleotide provided herein can form a complex between a polyribonucleotide and a target. In some embodiments, a polyribonucleotide forms a complex with a single target. In some embodiments, a circRNA forms a complex with a complex of two or more targets. In some embodiments, a polyribonucleotide forms a complex with a complex of three or more targets. In some embodiments, two or more polyribonucleotide form a complex with a single target. In some embodiments, two or more polyribonucleotide form a complex with two or more targets. In some embodiments, a first circRNA forms a complex with a first binding moiety of a first target and a second different binding moiety of a second target. In some embodiments, a first polyribonucleotide forms a complex with a first binding moiety of a first target and a second polyribonucleotide forms a complex with a second binding moiety of a second target.

In some embodiments, a polyribonucleotide can include a binding site for one or more binding moieties on one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some instances, a binding moiety is on a polypeptide, protein, or fragment thereof. In some embodiments, a binding moiety comprises a domain, a fragment, an epitope, a region, or a portion of a polypeptide, protein, or fragment thereof. For example, a binding moiety comprises a domain, a fragment, an epitope, a region, or a portion of an isolated polypeptide, a polypeptide of a cell, a purified polypeptide, or a recombinant polypeptide. For example, a binding moiety comprises a domain, a fragment, an epitope, a region, or a portion of an antibody or fragment thereof. For example, a binding moiety comprises a domain, a fragment, an epitope, a region, or a portion of a transcription factor. For example, a binding moiety comprises a domain, a fragment, an epitope, a region, or a portion of a receptor. For example, a binding moiety comprises a domain, a fragment, an epitope, a region, or a portion of a transmembrane receptor. Binding moieties may be on or comprise a domain, a fragment, an epitope, a region, or a portion of isolated, purified, and/or recombinant polypeptides. Binding moieties can include binding moieties on or that comprise a domain, a fragment, an epitope, a region, or a portion of a mixture of analytes (e.g., a lysate). For example, binding moieties are on or comprise a domain, a fragment, an epitope, a region, or a portion of from a plurality of cells or from a lysate of a single cell.

In some instances, a binding moiety is on or comprises a domain, a fragment, an epitope, a region, or a portion of a small molecule. For example, a binding moiety is on or comprises a domain, a fragment, an epitope, a region, or a portion of a drug. For example, a binding moiety is on or comprises a domain, a fragment, an epitope, a region, or a portion of a compound. For example, a binding moiety is on or comprises a domain, a fragment, an epitope, a region, or a portion of an organic compound. In some instances, a binding moiety is on or comprises a domain, a fragment, an epitope, a region, or a portion of a small molecule with a molecular weight of 900 Daltons or less. In some instances, a binding moiety is on or comprises a domain, a fragment, an epitope, a region, or a portion of a small molecule with a molecular weight of 500 Daltons or more. Binding moieties may be obtained, for example, from a library of naturally occurring or synthetic molecules, including a library of compounds produced through combinatorial means, e.g., a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A binding moiety can be on or comprise a domain, a fragment, an epitope, a region, or a portion of a member of a specific binding pair (e.g., a ligand). A binding moiety can be on or comprise a domain, a fragment, an epitope, a region, or a portion of monovalent (monoepitopic) or polyvalent (polyepitopic). A binding moiety can be antigenic or haptenic. A binding moiety can be on or comprise a domain, a fragment, an epitope, a region, or a portion of a single molecule or a plurality of molecules that share at least one common epitope or determinant site. A binding moiety can be on or comprise a domain, a fragment, an epitope, a region, or a portion of a part of a cell (e.g., a bacteria cell, a plant cell, or an animal cell). A binding moiety can be either in a natural environment (e.g., tissue), a cultured cell, or a microorganism (e.g., a bacterium, fungus, protozoan, or virus), or a lysed cell. A binding moiety can be modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

In some instances, a binding moiety is on or comprises a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render a binding moiety more readily detectable. Samples can include a quantity of a substance from a living thing or formerly living things. A sample can be natural, recombinant, synthetic, or not naturally occurring. A binding moiety can be any of the above that is expressed from a cell naturally or recombinantly, in a cell lysate or cell culture medium, an in vitro translated sample, or an immunoprecipitation from a sample (e.g., a cell lysate).

In some instances, a binding moiety of a target is expressed in a cell-free system or in vitro. For example, a binding moiety of a target is in a cell extract. In some instances, a binding moiety of a target is in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and μβ-microintaglio printing, and pMAC-protein microarray copying (See Kilb et al., Eng. Life Sci. 2014, 14, 352-364).

In some instances, a binding moiety of a target is synthesized in situ (e.g., on a solid substrate of an array) from a DNA template. In some instances, a plurality of binding moieties is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15): E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, a binding moiety of a nucleic acid target comprises a span of at least 6 nucleotides, for example, least 8, 9, 10, 12, 15, 20, 25, 30, 40, 50, or 100 nucleotides. In some instances, a binding moiety of a protein target comprises a contiguous stretch of nucleotides. In some instances, a binding moiety of a protein target comprises a non-contiguous stretch of nucleotides. In some instances, a binding moiety of a nucleic acid target comprises a site of a mutation or functional mutation, including a deletion, addition, swap, or truncation of the nucleotides in a nucleic acid sequence.

In some instances, a binding moiety of a protein target comprises a span of at least 6 amino acids, for example, least 8, 9, 10, 12, 15, 20, 25, 30, 40, 50, or 100 amino acids. In some instances, a binding moiety of a protein target comprises a contiguous stretch of amino acids. In some instances, a binding moiety of a protein target comprises a non-contiguous stretch of amino acids. In some instances, a binding moiety of a protein target comprises a site of a mutation or functional mutation, including a deletion, addition, swap, or truncation of the amino acids in a polypeptide sequence.

In some embodiments, a binding moiety is on or comprises a domain, a fragment, an epitope, a region, or a portion of a membrane bound protein. Exemplary membrane bound proteins include, but are not limited to, GPCRs (e.g., adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), receptor tyrosine kinases, receptor serine/threonine kinases, receptor guanylate cyclases, growth factor and hormone receptors (e.g., epidermal growth factor (EGF) receptor), and others. The binding moiety may also be on or comprise a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

In some embodiments, a polyribonucleotide can include other binding motifs for binding other intracellular molecules. Non-limiting examples of a polyribonucleotide, such as a circRNA, applications are listed in TABLE 1.

TABLE 1

| Process | Mode of Action (exemplary) |
|---|---|
| Directed Transcription | DNA-circRNA-Protein (pol, TF) |
| Epigenetic Remodeling | DNA-circRNA-Protein (SWI/SNF) |
| Transcriptional Interference | circRNA-DNA |
| Translational Interference | circRNA-mRNA or ribosome |
| Protein Interaction Inhibitor | circRNA-Protein |
| Protein Degradation | Protein-circRNA-Protein (ubiq) |
| RNA Degradation | RNA-circRNA-RNA (RNAse to RNA) |
| DNA Degradation | DNA-circRNA-Protein (DNA to DNAse) |
| Artificial Receptor | Cell Surface-circRNA-Substrate |
| Protein Translocation | Protein-circRNA-Protein/RNA |
| Cellular Fusion | Cell Surface-circRNA-Cell Surface |
| Complex Disassembly | Protein-circRNA-Protein/RNA |
| Receptor Inhibition | Protein-circRNA-Substrate |
| Signal Transduction | Protein-circRNA-Protein (caspase) |
| Multi-Enzyme Acceleration | Multiple Enzymes-circRNA |
| Induction of Receptor | circRNA-receptor |

In some embodiments, a polyribonucleotide described herein sequesters a target, e.g., DNA, RNA, proteins, and other cellular components to regulate cellular processes. A polyribonucleotide with binding sites for a target of interest can compete with binding of the target with an endogenous binding partner. In some embodiments, a polyribonucleotide described herein sequesters miRNA. In some embodiments, a polyribonucleotide described herein sequesters mRNA. In some embodiments, a polyribonucleotide described herein sequesters proteins. In some embodiments, a polyribonucleotide described herein sequesters ribosomes. In some embodiments, a polyribonucleotide described herein sequesters other a polyribonucleotides. In some embodiments, a polyribonucleotide described herein sequesters non-coding RNA, lncRNA, miRNA, tRNA, rRNA, snoRNA, ncRNA, siRNA, or shRNA. In some embodiments, a polyribonucleotide described herein includes a degradation element that degrades a sequestered target, e.g., DNA, RNA, protein, or other cellular component bound to the polyribonucleotide. Non-limiting examples of polyribonucleotide, such as circRNA, sequestration applications are listed in TABLE 2.

TABLE 2

| Process | Mode of Action (exemplary) |
|---|---|
| Transcriptional Interference | circRNA-DNA |
| Translational Intereference | circRNA-mRNA or ribosome |
| Protein Interaction Inhibitor | circRNA-Protein |
| microRNA sequester | circRNA-RNA (antisense) |
| circRNA sequester | circRNA-circRNA (antisense) |
| (endogenous circRNA) | |

In some embodiments, any of the methods of using a polyribonucleotide described herein can be in combination with a translating element. A polyribonucleotide described herein that comprises a translating element can translate RNA into proteins. For example, protein expression is facilitated by a polyribonucleotide comprising a sequence-specific RNA-binding motif, sequence-specific DNA-binding motif, protein-specific binding motif, and regulatory RNA motif. The regulatory RNA motif can initiate RNA transcription and protein expression.

### Encryptogen

As described herein, a polyribonucleotide can comprise an encryptogen to reduce, evade, or avoid the innate immune response of a cell. The polyribonucleotide can be present in either linear or circular form. In some embodiments, circular polyribonucleotides provided herein result in a reduced immune response from the host as compared to the response triggered by a reference compound, e.g., a circular polyribonucleotide producing a reduced immune response compared to a corresponding linear polynucleotide or a linear polynucleotide comprising an encryptogen, producing a reduced immune response compared to the corresponding linear polyribonucleotide lacking an encryptogen. In some embodiments, the polyribonucleotide has less immunogenicity than a counterpart lacking an encryptogen.

In some embodiments, the polyribonucleotide is non-immunogenic in a mammal, e.g., a human. In some embodiments, the polyribonucleotide is capable of replicating in a mammalian cell, e.g., a human cell.

In some embodiments, the polyribonucleotide includes sequences or expression products.

In some embodiments, the polyribonucleotide comprising the encryptogen has a half-life of at least that of a counterpart lacking the encryptogen. In some embodiments, the polyribonucleotide has a half-life that is increased over that of a counterpart. In some embodiments, the half-life is increased by about 5%, 10%, 15%, 20%, 25%, 30%, 35%, 40%, 45%, 50%, or greater. In some embodiments, the polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the polyribonucleotide comprising the encryptogen modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the polyribonucleotide comprising the encryptogen is at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs. In some embodiments, the polyribonucleotide comprising the encryptogen can be of a sufficient size to accommodate a binding site for a ribosome. One of skill in the art can appreciate that the maximum size of a polyribonucleotide comprising the encryptogen can be as large as is within the technical constraints of producing a polyribonucleotide, and/or using the polyribonucleotide.

In some embodiments, the circular polyribonucleotide comprising the encryptogen has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide comprising the encryptogen modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide comprising the encryptogen is at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs. In some embodiments, the circular polyribonucleotide can be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA can be produced from DNA and their 5′ and 3′ free ends annealed to produce a “string” of RNA, which ultimately can be circularized when only one 5′ and one 3′ free end remains. In some embodiments, the maximum size of a circular polyribonucleotide can be limited by the ability of packaging and delivering the RNA to a target. In some embodiments, the size of a circular polyribonucleotide is a length sufficient to encode useful polypeptides, and thus, lengths of less than about 20,000 base pairs, less than about 15,000 base pairs, less than about 10,000 base pairs, less than about 7,500 base pairs, or less than about 5,000 base pairs, less than about 4,000 base pairs, less than about 3,000 base pairs, less than about 2,000 base pairs, less than about 1,000 base pairs, less than about 500 base pairs, less than about 400 base pairs, less than about 300 base pairs, less than about 200 base pairs, less than about 100 base pairs can be useful.

### Cleavage Sequences

In some embodiments, the polyribonucleotide comprises at least one cleavage sequence. The polyribonucleotide can be present in either linear or circular form. In some embodiments, the cleavage sequence is adjacent to an expression sequence. In some embodiments, the polyribonucleotide comprises a cleavage sequence, such as in an immolating polyribonucleotide, a cleavable polyribonucleotide, or a self-cleaving polyribonucleotide. In some embodiments, the polyribonucleotide comprises two or more cleavage sequences, leading to separation of the polyribonucleotide into multiple products, e.g., miRNAs, linear RNAs, smaller circular polyribonucleotide, etc.

In some embodiments, the cleavage sequence comprises a ribozyme RNA sequence. A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

### Immolating Sequence

In some embodiments, a polyribonucleotide described herein comprises an immolating polyribonucleotide, cleavable polyribonucleotide, or self-cleaving polyribonucleotide. The polyribonucleotide can be present in either linear or circular form. A polyribonucleotide can deliver cellular components including, for example, RNA, lncRNA, lincRNA, miRNA, tRNA, rRNA, snoRNA, ncRNA, siRNA, or shRNA. In some embodiments, a polyribonucleotide comprises miRNA separated by (i) self-cleavable elements; (ii) cleavage recruitment sites; (iii) degradable linkers; (iv) chemical linkers; and/or (v) spacer sequences. In some embodiments, a polyribonucleotide comprises siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes. Non-limiting examples of polyribonucleotide, such as a circRNA, immodulation applications are listed in TABLE 3.

TABLE 3

| Process | Mode of Action (exemplary) |
|---|---|
| miRNA delivery | microRNAs in a circular form |
| | with self cleavage element (e.g., |
| | hammerhead), cleavage |
| | recruitment (e.g., ADAR) or |
| | degradable linker (glycerol) |
| siRNA delivery | siRNAs in a circular form with |
| | self cleavage element (e.g., |
| | hammerhead), cleavage |
| | recruitment (e.g., ADAR) or |
| | degradable linker (glycerol) |

### Riboswitches

In some cases, the polyribonucleotide comprises one or more riboswitches. The polyribonucleotide can be present in either linear or circular form.

A riboswitch is typically considered a part of the polyribonucleotide that can directly bind a small target molecule, and whose binding of the target affects RNA translation, the expression product stability and activity (Tucker B J, Breaker R R (2005), Curr Opin Struct Biol 15 (3): 342-8). Thus, the polyribonucleotide that comprises a riboswitch is directly involved in regulating its own activity, depending on the presence or absence of its target molecule. In some cases, a riboswitch has a region of aptamer-like affinity for a separate molecule. Thus, in the broader context of the instant invention, any aptamer included within a non-coding nucleic acid could be used for sequestration of molecules from bulk volumes. Downstream reporting of the event via “(ribo)switch” activity may be especially advantageous.

In some cases, the riboswitch may have an effect on gene expression including, but not limited to, transcriptional termination, inhibition of translation initiation, mRNA self-cleavage, and in eukaryotes, alteration of splicing pathways. The riboswitch may function to control gene expression through the binding or removal of a trigger molecule, such as by subjecting a polyribonucleotide that comprises the riboswitch to conditions that activate, deactivate or block the riboswitch to alter expression. Expression is altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule. Some examples of riboswitches are described herein.

In some cases, the riboswitch is a Cobalamin riboswitch (also B12-element), which binds adenosylcobalamin (the coenzyme form of vitamin B12) to regulate the biosynthesis and transport of cobalamin and similar metabolites.

In some cases, the riboswitch is a cyclic di-GMP riboswitches, which bind cyclic di-GMP to regulate a variety of genes. Two non-structurally related classes exist—cyclic di-GMP-1 and cyclic di-GMP-ll.

In some cases, the riboswitch is a FMN riboswitch (also RFN-element) which binds flavin mononucleotide (FMN) to regulate riboflavin biosynthesis and transport.

In some cases, the riboswitch is a glmS riboswitch, which cleaves itself when there is a sufficient concentration of glucosamine-6-phosphate.

In some cases, the riboswitch is a Glutamine riboswitches, which bind glutamine to regulate genes involved in glutamine and nitrogen metabolism. They also bind short peptides of unknown function. Such riboswitches fall into two classes, which are structurally related: the glnA RNA motif and Downstream-peptide motif.

In some cases, the riboswitch is a Glycine riboswitch, which binds glycine to regulate glycine metabolism genes. It comprises two adjacent aptamer domains in the same mRNA, and is the only known natural RNA that exhibits cooperative binding.

In some cases, the riboswitch is a Lysine riboswitch (also L-box), which binds lysine to regulate lysine biosynthesis, catabolism and transport.

In some cases, the riboswitch is a PreQ1 riboswitch, which binds pre-queuosine to regulate genes involved in the synthesis or transport of this precursor to queuosine. Two entirely distinct classes of PreGI riboswitches are known: PreQ1-1 riboswitches and PreQ1-11 riboswitches. The binding domain of PreQ1-1 riboswitches is unusually small among naturally occurring riboswitches. PreGI-II riboswitches, which are only found in certain species in the genera Streptococcus and Lactococcus, have a completely different structure, and are larger.

In some cases, the riboswitch is a Purine riboswitch, which binds purines to regulate purine metabolism and transport. Different forms of the purine riboswitch bind guanine (a form originally known as the G-box) or adenine. The specificity for either guanine or adenine depends completely upon Watson-Crick interactions with a single pyrimidine in the riboswitch at position Y74. In the guanine riboswitch, this residue is a cytosine (i.e. C74), in the adenine residue it is always a uracil (i.e. U74). Homologous types of purine riboswitches bind deoxyguanosine, but have more significant differences than a single nucleotide mutation.

In some cases, the riboswitch is a SAH riboswitch, which binds S-adenosylhomocysteine to regulate genes involved in recycling this metabolite which is produced when S-adenosylmethionine is used in methylation reactions.

In some cases, the riboswitch is a SAM riboswitch, which binds S-adenosyl methionine (SAM) to regulate methionine and SAM biosynthesis and transport. Three distinct SAM riboswitches are known: SAM-I (originally called S-box), SAM-II and the SMK box riboswitch. SAM-I is widespread in bacteria, but SAM-II is found only in α-, β- and a few γ-proteobacteria. The SMK box riboswitch is found only in the order Lactobacillales. These three varieties of riboswitch have no obvious similarities in terms of sequence or structure. A fourth variety, SAM-IV, appears to have a similar ligand-binding core to that of SAM-I, but in the context of a distinct scaffold.

In some cases, the riboswitch is a SAM-SAH riboswitch, which binds both SAM and SAH with similar affinities. Since they are always found in a position to regulate genes encoding methionine adenosyltransferase, it was proposed that only their binding to SAM is physiologically relevant.

In some cases, the riboswitch is a Tetrahydrofolate riboswitch, which binds tetrahydrofolate to regulate synthesis and transport genes.

In some cases, the riboswitch is a theophylline binding riboswitch or a thymine pyrophosphate binding riboswitch.

In some cases, the riboswitch is a T. tengcongensis glmS catalytic riboswitch, which senses glucosamine-6 phosphate (Klein and Ferre-D'Amare 2006).

In some cases, the riboswitch is a TPP riboswitch (also THI-box), which binds thiamine pyrophosphate (TPP) to regulate thiamine biosynthesis and transport, as well as transport of similar metabolites. It is the only riboswitch found so far in eukaryotes.

In some cases, the riboswitch is a Moco riboswitch, which binds molybdenum cofactor, to regulate genes involved in biosynthesis and transport of this coenzyme, as well as enzymes that use it or its derivatives as a cofactor.

In some cases, the riboswitch is a Adenine sensing add-A riboswitch, found in the 5′ UTR of the adenine deaminase encoding gene of Vibrio vulnificus.

### Aptazyme

In some cases, the polyribonucleotide comprises an aptazyme. Aptazyme is a switch for conditional expression in which an aptamer region is used as an allosteric control element and coupled to a region of catalytic RNA. In some cases, the aptazyme is active in cell type specific translation. In some cases, the aptazyme is active under cell state specific translation, e.g., virally infected cells or in the presence of viral nucleic acids or viral proteins. The polyribonucleotide can be present in either linear or circular form.

A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. More recently it has been shown that catalytic RNAs can be “evolved” by in vitro methods [1. Agresti J J, Kelly B T, Jaschke A, Griffiths A D: Selection of ribozymes that catalyse multiple-turnover Diels-Alder cycloadditions by using in vitro compartmentalization. Proc Natl Acad Sci USA 2005, 102:16170-16175; 2. Sooter L J, Riedel T, Davidson E A, Levy M, Cox J C, Ellington A D: Toward automated nucleic acid enzyme selection. Biological Chemistry 2001, 382(9):1327-1334.]. Winkler et al. have shown [Winkler W C, Nahvi A, Roth A, Collins J A, Breaker R R: Control of gene expression by a natural metabolite-responsive ribozyme. Nature 2004, 428:281-286] that, similar to riboswitch activity discussed above, ribozymes and their reaction products can regulate gene expression. In the context of the instant invention, it may be particularly advantageous to place a catalytic RNA or ribozyme within a larger non-coding RNA such that the ribozyme is present at many copies within the cell for the purposes of chemical transformation of a molecule from a bulk volume. Furthermore, encoding both aptamers and ribozymes in the same non-coding RNA may be particularly advantageous.

Some nonlimiting examples of ribozymes include hammerhead ribozyme, VL ribozyme, leadzyme, hairpin ribozyme.

In some cases, the aptazyme is a ribozyme that cleaves RNA sequences and which can be regulated as a result of binding ligand/modulator. The ribozyme may also be a self-cleaving ribozyme. As such, they combine the properties of ribozymes and aptamers. Aptazymes offer advantages over conventional aptamers due to their potential for activity in trans, the fact that they act catalytically to inactivate expression and that inactivation, due to cleavage of their own or heterologous transcript, is irreversible.

In some cases, the aptazyme is included in an untranslated region of the polyribonucleotide, e.g., linear or circular polyribonucleotide, and in the absence of ligand/modulator is inactive, allowing expression of the transgene. Expression can be turned off (or down-regulated) by addition of the ligand. It should be noted that aptazymes which are downregulated in response to the presence of a particular modulator can be used in control systems where upregulation of gene expression in response to modulator is desired.

Aptazymes may also permit development of systems for self-regulation of polyribonucleotide expression. For example, the protein product of the circular polyribonucleotide is the rate determining enzyme in the synthesis of a particular small molecule could be modified to include an aptazyme selected to have increased catalytic activity in the presence of that molecule, thereby providing an autoregulatory feedback loop for its synthesis. Alternatively, the aptazyme activity can be selected to be sensitive to accumulation of the protein product from the circular polyribonucleotide, or any other cellular macromolecule.

In some cases, the polyribonucleotide may include an aptamer sequence. Some nonlimiting examples include an RNA aptamer binding lysozyme, a Toggle-25t which is an RNA aptamer that includes 2′fluoropyrimidine nucleotides bind thrombins with high specificity and affinity, RNATat that binds human immunodeficiency virus trans-acting responsive element (HIV TAR), RNA aptamer-binding hemin, RNA aptamer-binding interferon γ, RNA aptamer binding vascular endothelial growth factor (VEGF), RNA aptamer binding prostate specific antigen (PSA), RNA aptamer binding dopamine, and RNA aptamer binding the non-classical oncogene, heat shock factor 1 (HSF1).

### Other Reagents

In some cases, the composition or pharmaceutical composition comprises reagents besides the alcohol or the cell-penetrating agent and the polyribonucleotide. For example, the composition or pharmaceutical composition can further comprise one or more active agents, e.g., therapeutic agents, besides the polyribonucleotide, such as one or more peptides, nucleic acids (e.g., DNA), proteins (e.g., antibodies or fragments thereof), APCs, viruses, small molecule compounds, prodrugs, or a combination thereof. In some cases, the composition or pharmaceutical composition comprising one or more active agents, e.g., therapeutic agents, can be formulated using one or more physiologically acceptable carriers, comprising excipients, diluents, and/or auxiliaries, e.g., which facilitate processing of the one or more active agents into preparations that can be administered. The polyribonucleotide can be present in either linear or circular form.

In some cases, the composition or pharmaceutical composition described herein can further comprise therapeutically acceptable excipients, carriers, diluents, adjuvants, other auxiliary vehicles, buffers, stabilizers, or scaffolds. The the composition or pharmaceutical composition can further comprise other reagents to maintain appropriate physical or chemical properties, such as, but not limited to, salt concentration, osmolality, pH, hydrophobicity/hydrophility, and solubility. For example, the composition can comprise a stabilizer, e.g., glucose at an appropriate concentration, e.g., about 4.5 g/L. In some embodiments, the polyribonucleotide is non-immunogenic. In some embodiments, the polyribonucleotide is immunogenic. A composition or pharmaceutical composition can further comprise appropriate adjuvant(s) or other agents that can enhance or depress the immunogenicity of the polyribonucleotide.

Non-limiting examples of the therapeutically acceptable carriers include starch, glucose, lactose, sucrose, gelatin, saline, gum acacia, keratin, urea, malt, rice flour, chalk, silica gel, sodium stearate, glycerol monostearate, talc, sodium chloride, dried skim milk, glycerol, propylene, glycol, humectants (e.g., urea), glycols (e.g., propylene glycol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, polymers such as polyethylene glycols, and water. If desired, the carrier can also contain minor amounts of wetting or emulsifying agents, or pH buffering agents.

### Stability

The polyribonucleotide provided herein can be stable after delivery into a cell or tissue, allowing for translation of the polyribonucleotide resulting in a biological effect on the cell or tissue in which the polyribonucleotide was delivered. The polyribonucleotide can be present in either linear or circular form. In some embodiments, the polyribonucleotide is a linear polyribonucleotide and is used for a short term biological effect, such as for a biological effect that lasts at least 1 hour, 6 hours, 12 hours, 24 hours, 48 hours, 72 hours, or any time therebetween. In some embodiments, the polyribonucleotide is a circular polyribonucleotide and is used for a long term biological effect, such as a biological effect that last for at least 3 days, 4 days, 5 days, 6 days, 7 days, 8 days, 9 days, 10 days, 11 days, 12 days, 13 days, 14 days, or more, or any time therebetween. In some embodiments, the polyribonucleotide is substantially resistant to degradation, e.g., exonuclease. In some embodiments, the polyribonucleotide is resistant to self-degradation. In some embodiments, the polyribonucleotide lacks an enzymatic cleavage site, e.g., a dicer cleavage site.

In some embodiments, the polyribonucleotide persists in a cell during cell division. In some embodiments, the polyribonucleotide persists in daughter cells after mitosis. In some embodiments, the polyribonucleotide is replicated within a cell and is passed to daughter cells. In some embodiments, the polyribonucleotide comprises a replication element that mediates self-replication of the polyribonucleotide. In some embodiments, the replication element mediates transcription of the circular polyribonucleotide into a linear polyribonucleotide that is complementary to the circular polyribonucleotide (linear complementary). In some embodiments, the linear complementary polyribonucleotide can be circularized in vivo in cells into a complementary circular polyribonucleotide. In some embodiments, the complementary polyribonucleotide can further self-replicate into another circular polyribonucleotide, which has the same or similar nucleotide sequence as the starting circular polyribonucleotide. One exemplary self-replication element includes HDV replication domain (as described by Beeharry et al, Virol, 2014, 450-451:165-173). In some embodiments, a cell passes at least one polyribonucleotide to daughter cells with an efficiency of at least 25%, 50%, 60%, 70%, 80%, 85%, 90%, 95%, or 99%. In some embodiments, cell undergoing meiosis passes the polyribonucleotide to daughter cells with an efficiency of at least 25%, 50%, 60%, 70%, 80%, 85%, 90%, 95%, or 99%. In some embodiments, a cell undergoing mitosis passes the polyribonucleotide to daughter.

### Compositions and Delivery Routes

A composition or pharmaceutical composition provided herein can be formulated based, in part, on the intended route of administration of the composition. In certain aspects of the disclosure, a composition or pharmaceutical composition provided herein can be administered topically at or near particular sites of a subject. The composition can be as described herein and further comprise a pharmaceutically acceptable excipient. Direct topical application, e.g., of a viscous liquid, solution, suspension, gel, jelly, cream, lotion, ointment, suppository, foam, or aerosol spray, can be used for local administration. In some embodiments, the composition is formulated for systemic administration. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include, if needed, preservatives (e.g., p-hydroxybenzoic acid esters) and/or antioxidants (e.g., ascorbic acid and tocopherol). The polyribonucleotide can be present in either linear or circular form.

The composition or pharmaceutical composition can comprise an alcohol (e.g., ethanol) and the polyribonucleotide. The composition or pharmaceutical composition can comprise a cell-penetrating agent and the polyribonucleotide. The composition or pharmaceutical composition can comprise a diluent and the polyribonucleotide, wherein the composition or pharmaceutical composition is free of any carrier.

A composition or pharmaceutical composition provided herein can be formulated for direct administration onto a surface area of a subject, such as skin, nails, surface areas of oral cavity, nasal cavity, ear cavity, vaginal, cervical, inter uterine, urinary tract, and eye.

A composition or pharmaceutical composition described herein can be a liquid preparation such as a suspension, syrup, or elixir. In some cases, aqueous solutions are packaged for use as is, or lyophilized, and the lyophilized preparation being combined with a sterile solution prior to administration. The composition can be delivered as a solution or as a suspension. In general, formulations such as a gel (e.g., DMSO gel), jellies, creams, lotions (e.g., Johnson & Johnson lotion), suppositories and ointments can provide an area with more extended exposure to one or more active agents, while formulations in solution, e.g., sprays, can provide more immediate, short-term exposure.

A composition or pharmaceutical composition as described herein can have a pH of about 7. In some embodiments, the composition or pharmaceutical composition has a viscosity about the same as water. The composition or pharmaceutical composition can be substantially free of hydrophobic or lipophilic groups. In some embodiments, the composition or pharmaceutical compositions is substantially free of hydrocarbons. The composition or pharmaceutical composition can be substantially free of fatty acids, lipids, liposomes, cholesterol, or any combination thereof.

In some embodiments, a method of delivery of the polyribonucleotides as described herein comprises topically applying a composition comprising a mixture of a polyribonucleotide and ethanol to a surface area of a subject, wherein the ethanol constitutes at least about 0.3% v/v to about 75% v/v of the mixture. In some embodiments, a method of delivery of the polyribonucleotides as described herein comprises topically applying a composition comprising a mixture of a polyribonucleotide and alcohol to a surface area of a subject, wherein the alcohol constitutes at least about 0.3% v/v to about 75% v/v of the mixture. In some embodiments, a method of delivery of the polyribonucleotides as described herein comprises topically applying a composition comprising a mixture of a polyribonucleotide and a cell-penetrating agent to a surface area of a subject, wherein the cell penetrating agent constitutes at least about 0.3% v/v to about 75% v/v of the mixture. In some embodiments, the ethanol, alcohol, or cell-penetrating agent constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture, or any percentage v/v therebetween. In some embodiments, the ethanol, alcohol, or cell-penetrating agent at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture, or any percentage v/v therebetween.

Often, the composition or the pharmaceutical composition as described herein delivers the polyribonucleotide to a dermal or epidermal tissue of a subject. In some embodiments, the polyribonucleotides are delivered without iontophoresis.

### Percutaneous Administration

Formulations for topical administration can take the form of a liquid, a semisolid dosage form (e.g., a paste, a cream, a lotion, a powder, an ointment or a gel), a patch, a film, or a spray. In some cases, the topical composition can be a cream or gel that can be applied to an affected area of the skin of a subject in need thereof (e.g., percutaneous or dermal administration). Different release profiles can be achieved with different forms, such as but not limited to controlled release, delayed release, extended release, or sustained release. The topical pharmaceutical composition can be applied multiple times a day, once per day, or as often as needed. The polyribonucleotide can be present in either linear or circular form.

In some cases, the composition or pharmaceutical composition is be formulated for direct application on a skin area (e.g., percutaneous or dermal administration). A composition or pharmaceutical composition provided herein can comprise a dermatologically acceptable diluent. Such diluents are compatible with skin, nails, mucous membranes, tissues, and/or hair, and can include any dermatological diluent meeting these requirements. Such diluents can be readily selected by one of ordinary skill in the art. In formulating skin ointments, one or more agents can be formulated in an oleaginous hydrocarbon base, an anhydrous absorption base, a water-in-oil absorption base, an oil-in-water water-removable base and/or a water-soluble base.

Ointments and creams are, for example, be formulated with an aqueous or oily base with the addition of suitable thickening and/or gelling agents. Lotions can be formulated with an aqueous or oily base and will in general also comprise one or more emulsifying agents, stabilizing agents, dispersing agents, suspending agents, thickening agents, or coloring agents. The construction and use of transdermal patches for the delivery of pharmaceutical agents is known in the art. Such patches can be constructed for continuous, pulsatile, or on demand delivery of pharmaceutical agents.

Lubricants which can be used to form compositions and dosage forms can comprise calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the composition.

A composition or pharmaceutical composition provided herein can be in any form suitable for topical administration, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. Other than the polyribonucleotides and other active ingredient(s), the amount of the various constituents of the compositions provided herein can be those used in the art. These compositions can constitute protection, treatment or care creams, milks, lotions, gels or foams for the face, for the hands, for the body and/or for the mucous membranes, or for cleansing the skin. The compositions can also consist of solid preparations constituting soaps or cleansing bars.

A composition or pharmaceutical composition provided herein for local/topical administration can comprise one or more antimicrobial preservatives such as quaternary ammonium compounds, organic mercurials, p-hydroxy benzoates, aromatic alcohols, chlorobutanol, and the like.

### Inhalation (e.g., Nasal Administration or Oral Inhalation)

A composition or pharmaceutical composition described herein can be formulated for administration via the nasal passages of a subject. Formulations suitable for nasal administration, wherein the carrier is a solid, can include a coarse powder having a particle size, for example, in the range of about 10 to about 500 microns which can be administered in the manner in which snuff is taken, e.g., by rapid inhalation through the nasal passage from a container of the powder held close up to the nose. The formulation can be a nasal spray, nasal drops, or by aerosol administration by nebulizer. The formulation can comprise aqueous or oily solutions of the polyribonucleotide. The polyribonucleotide can be present in either linear or circular form.

A composition or pharmaceutical composition provided herein can be formulated as an aerosol formulation. The aerosol formulation can be, e.g., an aerosol solution, suspension or dry powder. The aerosol can be administered through the respiratory system or nasal passages. For example, the composition can be suspended or dissolved in an appropriate carrier, e.g., a pharmaceutically acceptable propellant, and administered directly into the lungs using a nasal spray or inhalant. For example, an aerosol formulation comprising one or more active agents is dissolved, suspended or emulsified in a propellant or a mixture of solvent and propellant, e.g., for administration as a nasal spray or inhalant. The aerosol formulation can comprise any acceptable propellant under pressure, such as pharmaceutically acceptable propellant.

An aerosol formulation for nasal administration can be an aqueous solution designed to be administered to the nasal passages in drops or sprays. Nasal solutions can be similar to nasal secretions in that they can be isotonic and slightly buffered to maintain a pH of about 5.5 to about 6.5. In some cases, pH values outside of this range can be used.

An aerosol formulation for inhalation can be designed so that one or more active agents are carried into the respiratory system, e.g., along the respiratory tract, e.g., nasal cavity, mouth, pharynx, larynx, trachea, primary bronchi, and lungs, of the subject when administered by the nasal or oral respiratory route. Inhalation solutions are administered, for example, by a nebulizer. Inhalations or insufflations, comprising finely powdered or liquid compositions, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

The aerosol formulation can also include other components, for example, surfactants or other components, such as oils and detergents. These components can serve to stabilize the formulation and/or lubricate valve components.

The aerosol formulation can be packaged under pressure and can be formulated as an aerosol using solutions, suspensions, emulsions, powders, and semisolid preparations. For example, a solution aerosol formulation can include a solution of an active agent such as in (substantially) pure propellant or as a mixture of propellant and solvent. The solvent can be used to dissolve one or more active agents and/or retard the evaporation of the propellant. Solvents can include, for example, water, and glycols. Any combination of suitable solvents can be use, optionally combined with preservatives, antioxidants, and/or other aerosol components.

An aerosol formulation can be a dispersion or suspension. A suspension aerosol formulation can comprise a suspension of one or more active agents, e.g., polyribonucleotides, and a dispersing agent. Dispersing agents can include, for example, sorbitan trioleate, oleyl alcohol, oleic acid, lecithin, and corn oil. A suspension aerosol formulation can also comprise lubricants, preservatives, antioxidant, and/or other aerosol components.

An aerosol formulation can also be formulated as an emulsion. An emulsion aerosol formulation can comprise, for example, a surfactant, water, and a propellant, as well as an active agent or combination of active agents, e.g., one or more peptides. The surfactant used can be nonionic, anionic, or cationic. One example of an emulsion aerosol formulation comprises, for example, surfactant, water, and propellant. Another example of an emulsion aerosol formulation comprises, for example, vegetable oil, glyceryl monostearate, and propane.

### Oral Administration

In some cases, a composition or pharmaceutical composition provided herein can be formulated for oral administration. Sometimes, the composition or pharmaceutical composition can include a scaffold (e.g., pills, dragees, capsules, lozenges, hard candy, liquids, gels, syrups, slurries, powders, suspensions, elixirs, wafers) comprising a mixture of a cell-penetrating agent and a polyribonucleotide. In some cases, the scaffold is configured to release the mixture anywhere along the gastrointestinal tract. In other cases, the scaffold is configured to release the mixture at a certain location of the gastrointestinal tract, for instance, one or more locations of pharynx, esophagus, stomach, intestine, or colon. Different release profiles can be achieved with different scaffolds for oral administration, such as but not limited to controlled release, delayed release, extended release, or sustained release. The polyribonucleotide can be present in either linear or circular form.

For oral administration, a composition or pharmaceutical composition provided herein can be formulated readily by combining the mixture with pharmaceutically acceptable diluents known in the art. Such diluents enable active agents to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can include pharmaceutically acceptable diluents including solid diluents or fillers, sterile aqueous media and various non-toxic organic solvents. A solid diluent can be one or more substances which can also act as diluents, flavoring agents, solubilizers, lubricants, suspending agents, binders, preservatives, tablet disintegrating agents, or an encapsulating material.

Liquid mixture for oral use can be formulated in capsules which can comprises the mixture with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like. Oils or non-aqueous solvents can be required to bring the one or more active agents into solution, due to, for example, the presence of large lipophilic moieties. Alternatively, emulsions, suspensions, or other preparations can be used.

Pharmaceutical preparations that can be used orally include push-fit capsules made of gelatin, as well as soft, sealed capsules made of gelatin and a plasticizer, such as glycerol or sorbitol. The push-fit capsules can comprise the mixture of the cell-penetrating agent and the polyribonucleotide in admixture with filler such as lactose, binders such as starches, and/or lubricants such as talc or magnesium stearate and, optionally, stabilizers. Soft capsules can comprise excipients such as fatty oils, liquid paraffin, or liquid polyethylene glycols. In addition, stabilizers can be added. All formulations for oral administration can be in dosages suitable for administration.

When formulating compounds for oral administration, it can be desirable to utilize gastroretentive formulations to enhance absorption from the gastrointestinal tract. A formulation which is retained in the stomach for several hours can release a mixture of the cell-penetrating agent and polyribonucleotide slowly and provide a sustained release that can be used herein. A formulation which is retained in the stomach for several hours can release a mixture of the alcohol and polyribonucleotide slowly and provide a sustained release that can be used herein. Expandable, floating and bioadhesive techniques can be utilized to maximize application of the mixture to the surface area of the gastrointestinal tract.

### Ophthalmic Administration

A composition or pharmaceutical composition provided herein can be administered through eyes, e.g. delivered in eye drops or ointment. The topical application of the composition to eyes can contact the cells in eyes, for instance, retina, with the mixture of the cell-penetrating agent and the polyribonucleotide. The topical application of the composition to eyes can contact the cells in eyes, for instance, retina, with the mixture of the alcohol (e.g., ethanol) and the polyribonucleotide. The polyribonucleotide can be present in either linear or circular form.

Eye drops can be prepared by the mixture of the cell-penetrating agent and the polyribonucleotide alone, if the mixture is in liquid phase itself. Eye drops can be prepared by the mixture of the alcohol (e.g., ethanol) and the polyribonucleotide alone, if the mixture is in liquid phase itself. Alternatively, eye drops can be prepared by dissolving a solid mixture of the cell-penetrating agent and the polyribonucleotide in a sterile aqueous solution such as physiological saline, buffering solution, etc., or by combining powder compositions to be dissolved before use. Alternatively, eye drops can be prepared by dissolving a solid mixture of the alcohol (e.g., ethanol) and the polyribonucleotide in a sterile aqueous solution such as physiological saline, buffering solution, etc., or by combining powder compositions to be dissolved before use. Other vehicles can be chosen, as is known in the art, including but not limited to: balance salt solution, saline solution, water soluble poly ethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose, or other agents known to those skilled in the art).

### Other Administration

In some cases, a composition or pharmaceutical composition described herein is formulated for administration as a suppository. For rectal application, suitable dosage forms for a composition include suppositories (emulsion or suspension type), and rectal gelatin capsules (solutions or suspensions). In an exemplary suppository formulation, the composition provided herein are combined with an appropriate pharmaceutically acceptable suppository base such as cocoa butter, esterified fatty acids, glycerinated gelatin, and various water-soluble or dispersible bases like polyethylene glycols. Various additives, enhancers, or surfactants can be incorporated. For example, a low melting wax, such as a mixture of triglycerides, fatty acid glycerides, Witepsol S55 (trademark of Dynamite Nobel Chemical, Germany), or cocoa butter can be first melted and the mixture can be dispersed homogeneously, for example, by stirring. The molten homogeneous mixture can then be poured into convenient sized molds, allowed to cool, and to solidify. The polyribonucleotide can be present in either linear or circular form.

In some cases, a composition or pharmaceutical composition described herein is formulated for mucosal administration.

In some cases, a composition or pharmaceutical composition described herein is formulated for vaginal administration. In some cases, pessaries, tampons, creams, gels, pastes, foams, or sprays contain one or compositions described herein.

In some cases, a composition or pharmaceutical composition is formulated for administration to epithelial cells. This composition or pharmaceutical composition can be free of any carrier and comprises a diluent and the polyribonucleotide as described herein. In some embodiments, this composition or pharmaceutical composition is directly applied to epithelial cells for the delivery of the polyribonucleotide.

### Pre-Treatment

A composition or pharmaceutical composition as described herein can be applied to a surface after application of a sterilizing agent to that surface (e.g., pre-treatment of the surface with a sterilizing agent for delivery of the polyribonucleotide). The surface can be a surface area of a subject. The surface area of the subject comprises cells, such as epithelial cells.

A sterilizing agent can be any agent that is bactericidal, bacteriostatic, and/or actively kills microorganisms, inactivates microorganism, or prevents microorganisms from growing. In some embodiments, the sterilizing agent is an alcohol, iodine, or hydrogen peroxide. The sterilizing agent can be UV light or laser light. In some embodiments, the sterilizing agent is heat delivered electrically or through other means, such as by vapor or contact.

The sterilizing agent can be applied to the surface area of the subject by various non-invasive methods. For example, a sterilizing agent can be applied by a wipe or swab comprising the sterilizing agent. In some embodiments, the sterilizing agent is applied as a spray. Various devices can be used to apply the sterilizing agent. For example, a device that produces UV light or laser light can be used. In other embodiments, a device that produces heat can be used.

The composition or pharmaceutical composition applied to the surface area after pre-treatment can be free of any carrier and comprise the polyribonucleotide and a diluent. The polyribonucleotide can be a linear polyribonucleotide or a circular polyribonucleotide.

### Preservatives

A composition or pharmaceutical composition provided herein can comprise material for a single administration, or can comprise material for multiple administrations (e.g., a “multidose” kit). The polyribonucleotide can be present in either linear or circular form. The composition or pharmaceutical composition can include one or more preservatives such as thiomersal or 2-phenoxyethanol. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In ophthalmic products, e.g., such preservatives can be employed at a level of from 0.004% to 0.02%. In the compositions described herein the preservative, e.g., benzalkonium chloride, can be employed at a level of from 0.001% to less than 0.01%, e.g., from 0.001% to 0.008%, preferably about 0.005% by weight.

Polyribonucleotides can be susceptible to RNase that can be abundant in ambient environment. Compositions provided herein can include reagents that inhibit RNase activity, thereby preserving the polyribonucleotide from degradation. In some cases, the composition or pharmaceutical composition includes any RNase inhibitor known to one skilled in the art. Alternatively or additionally, the polyribonucleotide, and cell-penetrating agent and/or pharmaceutically acceptable diluents or carriers, vehicles, excipients, or other reagents in the composition provided herein can be prepared in RNase-free environment. The composition can be formulated in RNase-free environment.

In some cases, a composition provided herein can be sterile. The composition can be formulated as a sterile solution or suspension, in suitable vehicles, known in the art. The composition can be sterilized by conventional, known sterilization techniques, e.g., the composition can be sterile filtered.

### Salts

In some cases, a composition or pharmaceutical composition provided herein comprises one or more salts. For controlling the tonicity, a physiological salt such as sodium salt can be included a composition provided herein. Other salts can comprise potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can comprise those of the inorganic ions, such as, for example, sodium, potassium, calcium, magnesium ions, and the like. Such salts can comprise salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can comprise one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/Surfactants

A composition or pharmaceutical composition provided herein can comprise one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, e.g., octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Effective Delivery

Compositions, pharmaceutical compositions, methods, and kits provided herein can offer an easy-to-operate and effective solution for delivery of polyribonucleotides into cells. In some cases, the delivery of polyribonucleotides into cells is therapeutic. In some cases, the delivery efficiency can be relatively high in the presence of the alcohol described herein as compared to delivery without the alcohol. In some cases, the delivery efficiency can be relatively high in the presence of the cell-penetrating agent described herein as compared to delivery without the cell-penetrating agent. In some cases, the delivery efficiency can be relatively high after pre-treatment using a sterilizing agent (e.g., alcohol) described herein as compared to delivery without the pre-treatment. The polyribonucleotide can be present in either linear or circular form.

In some cases, the delivery efficiency is expressed as a ratio of the amount of the polyribonucleotides that are delivered into a cell over the amount of the total polyribonucleotides that are brought into contact with the cell. In some cases, the delivery efficiency is expressed as a ratio of the amount of the polyribonucleotides that are delivered into cells over the amount of the total polyribonucleotides that are administered near the cells (for instance, the amount delivered into skin cells when the polyribonucleotides are applied directly on a skin area). The delivery efficiency of the methods provided herein can be at least about 0.5%, 1%, 1.5%, 2%, 3%, 4%, 5%, 10%, 15%, 20%, 25%, 30%, 35%, 40%, 45%, 50%, 55%, 60%, 65%, 70%, 75%, 80%, 85%, 90%, or 95%. In some cases, the delivery efficiency of the methods provided herein can be about 0.5%, 1%, 1.5%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%.

In some cases, the pharmaceutical effects of the compositions and methods provided herein are measured in terms of the biological effects observed from the subject after the administration. For example, the abundance of the polyribonucleotides in the subject or one or more cells of the subject (e.g., blood sample or tissue biopsy) can be measured. In other cases, expression product can be taken as an indicator of the delivery efficiency of the compositions and methods provided herein, if the compositions include expression sequence that encodes a protein to be expressed in the subject.

The compositions and methods provided herein can be particularly more effective for delivery of circular polyribonucleotide as compared to linear polyribonucleotide. An amount of the circular polyribonucleotide delivered to a cell is at least 1.1, 1.2, 1.3, 1.4, 1.5, 1.6, 1.7, 1.8, 1.9, 2.0, 2.2, 2.4, 2.5, 2.6, 2.8, 3.0, 3.5, 4.0, 4.5, 5.0, 6.0, 7.0, 8.0, 9.0, or 10.0 fold higher than an amount of a linear polyribonucleotide contacted to the cell with a mixture comprising the linear polyribonucleotide and the cell-penetrating agent. In some cases, an amount of the circular polyribonucleotide delivered to a cell is at least 1.1 fold higher than an amount of a linear polyribonucleotide contacted to the cell with a mixture comprising the linear polyribonucleotide and the cell-penetrating agent.

When administered in vivo, a polyribonucleotide can be delivered, according to methods provided herein, into any type of cells that are in proximity to a surface area of the subject, depending on the administration route. For example, polyribonucleotides can be delivered to epithelial cells that are located under skin, on the surface of cavities or tracts (e.g., mouth, nasal cavity, throat, GI tract, respiratory tract, or vagina) by the methods provided herein. Non-limiting types of epithelial cell include simple squamous epithelium, simple cuboidal epithelium, simple columnar epithelium, pseudostratified columnar epithelium, stratified squamous epithelium, stratified cuboidal epithelium, stratified columnar epithelium, and transitional epithelium. Polyribonucleotides can be delivered to any type of cells under the surface area, for example, skin, by the methods provided herein, including, but not limited to, keratinocytes, Merkel cells, melanocytes, Langerhans cells, fibroblasts, macrophages, and adipocytes. Polyribonucleotides can be delivered to any part of the tissue underneath skin by the methods provided herein, such as, epidermis, basement membrane, dermis, and subcutaneous tissue.

For example, the polyribonucleotides can get into blood vessels in proximity to the administration location and/or blood. For example, a polyribonucleotide can be delivered into epithelial cells in the capillary wall underneath a skin. In some cases, the delivery is systemic, e.g., the polyribonucleotide is delivered inside the blood vessel and transfected into the blood cells, such as, red blood cells, white blood cells, and platelets. In these cases, the polyribonucleotide can be delivered into circulating cells which can spread into any part of the body, which can be recognized as a systemic delivery of the polyribonucleotide.

The compositions, pharmaceutical compositions, methods, and kits provided herein can be suitable for extended delivery of the polyribonucleotides. For example, the polyribonucleotide and alcohol (e.g., ethanol) can be formulated for extended release, controlled release, delayed release, or sustained release, so that particular therapeutic effect can be achieved or the polyribonucleotide can be delivered into desired locations of the subject. For instance, the polyribonucleotide and alcohol can be formulated in a form of a patch that is to be adhered to a skin area of a subject for an extended period, e.g., at least about 2 hrs, 4 hrs, 6 hrs, 8 hrs, 10 hrs, 15 hrs, 20 hrs, 24 hrs, 36 hrs, 2 days, 3 days, 4 days, 5 days, 6 days, 7 days, 8 days, 9 days, 10 days, 11 days, 12 days, 13 days, 2 weeks, 3 weeks, 4 weeks, 5 weeks, 2 months, 3 months, 4 months, or even longer. In some cases, the polyribonucleotide and alcohol in the patch are delivered over an extended period, e.g., as long as the patch is adhered on the skin area. In other embodiments, the polyribonucleotide and cell-penetrating agent can be formulated for extended release, controlled release, delayed release, or sustained release, so that particular therapeutic effect can be achieved or the polyribonucleotide can be delivered into desired locations of the subject. For instance, the polyribonucleotide and cell-penetrating agent can be formulated in a form of a patch that is to be adhered to a skin area of a subject for an extended period, e.g., at least about 2 hrs, 4 hrs, 6 hrs, 8 hrs, 10 hrs, 15 hrs, 20 hrs, 24 hrs, 36 hrs, 2 days, 3 days, 4 days, 5 days, 6 days, 7 days, 8 days, 9 days, 10 days, 11 days, 12 days, 13 days, 2 weeks, 3 weeks, 4 weeks, 5 weeks, 2 months, 3 months, 4 months, or even longer. In some cases, the polyribonucleotide and cell-penetrating agent in the patch are delivered over an extended period, e.g., as long as the patch is adhered on the skin area.

In some cases, the compositions, pharmaceutical compositions, methods, and kits provide polyribonucleotides that are delivered into a cell and have biological effects over an extended period of time. For example, some polyribonucleotides have little to no susceptibility to RNase or they can have very long half-life inside the cell are used. As a result, the polyribonucleotides can be present and potentially active throughout an extended period of time, for instance, at least about 24 hrs, 36 hrs, 2 days, 3 days, 4 days, 5 days, 6 days, 7 days, 8 days, 9 days, 10 days, 11 days, 12 days, 13 days, 2 weeks, 3 weeks, 4 weeks, 5 weeks, 2 months, 3 months, 4 months, 5 months, 6 months, 7 months, 8 months, 9 months, 10 months, 1 year, 2 years, 3 years, or even longer. In some cases, the polyribonucleotides can have a half-life that is about about 24 hrs, 36 hrs, 2 days, 3 days, 4 days, 5 days, 6 days, 7 days, 8 days, 9 days, 10 days, 11 days, 12 days, 13 days, 2 weeks, 3 weeks, 4 weeks, 5 weeks, 2 months, 3 months, 4 months, 5 months, 6 months, 7 months, 8 months, 9 months, 10 months, 1 year, 2 years, or 3 years. In certain cases, the biological effects the polyribonucleotides extend over the lifetime of the cell or subject, for instance, when the polyribonucleotides are administered for gene editing purposes in cells having lifetime longevity or capable of producing progeny cells (e.g., stem cells or other progenitor cells).

### Kits and Application Tools

Depending on the different intended administration routes, the composition provided herein can be packaged in different manners, and/or in some cases, in different kits that include the composition and one or more application tools configured to administer the composition to a subject via the intended routes. As discussed above, a composition provided herein can be formulated for administered through different routes, including direct topic administration (e.g., percutaneous, suppository, mucosal, and intravaginal), inhalation, and oral ingestion. In aspects, the present disclosure provides kits for administration of the compositions comprising the polyribonucleotide and the cell-penetrating agent. In aspects, the present disclosure provides kits for administration of the compositions comprising the polyribonucleotide and alcohol. In aspects, the present disclosure provides kits for administration of the compositions comprising the polyribonucleotide and ethanol. In aspects, the present disclosure provides kits for administration of the compositions free of any carrier comprising the polyribonucleotide and diluent after pre-treatment as described herein. The polyribonucleotide can be present in either linear or circular form.

A kit can be configured for direct topical administration, e.g., direct application on skin. In some cases, the kit comprises a substrate or scaffold comprising a composition as provided herein. The substrate or scaffold can be in a form of a patch, a wipe, Q-tip, or any other form that allows direct application of the composition onto a subject's skin surface. The substrate or scaffold can be made of disposable material, or biodegradable materials. In some cases, the substrate can be a fiber layer, e.g., a fiber layer constituted using a non-elastomer raw material and having elongatability at least in one direction, e.g., cotton, eucalyptus or biocellulose. As the fiber layer, paper, a nonwoven fabric, a woven fabric and so forth can be utilized. The fiber layer can be hydrophilic and have liquid retention properties. The fiber layer can be made of hydrophilic fibers obtained using a hydrophilic raw material, and from non-elastomer raw material or the fibers formed therefrom. In some cases, the kit includes a liquid composition provided herein and a transfer tool configured to transfer or dispense the liquid composition. The transfer tool can be simple as a straw, an aurilave, or a transfer pipette. The transfer tool can also be designed with additional features, such as graduation, actuation, alarm system, or automatic dispensing system.

A kit can be configured for inhalatory administration of the composition provided herein. In some cases, the kit includes an inhaler and the composition. In some cases, the inhaler is a nebulizer configured to convert liquid composition into aerosol. The composition can be packaged in liquid form, or in solid form ready to be dissolved in solvent for aerosolization and inhalation. Non-limiting examples of nebulizers include breath activated or breath-actuated nebulizers, hand-held inhaler devices, jet nebulizers, vibrating mesh nebulizers, nebulizers as described in international patent application No. WO 2004/071368, U.S. Published application Nos. 2004/0011358 and 2004/0035413, each of which is incorporated herein by reference in its entirety. The nebulizer can have a compressed air source. In some cases, the nebulizer converts liquid medication into an aerosol by extruding the pharmaceutical preparation through micron or submicron-sized holes, or by applying ultrasonic waves. In some cases, the inhaler is a dry powder aerosolization device which converts a solid composition into aerosol. Dry powder aerosolization device can be a dry powder inhaler, such as an active or passive dry powder inhaler. Exemplary dry powder inhalers include those as described in U.S. Pat. Nos. 4,069,819 and 4,995,385, 3,991,761, 3,991,761, each of which is incorporated herein by reference in its entirety.

A kit can be configured for oral administration of the composition provided herein. The composition for oral administration, e.g., oral ingestion, can be formulated in various forms such as tablets, including chewable tablets, pills, dragees, capsules, lozenges, hard candy, liquids, gels, syrups, slurries, powders, suspensions, elixirs, wafers, and the like.

A kit provided herein can further comprise a container, such as a bottle, box, capsule, or dispenser, containing the formulated composition. A container as disclosed can be an application tool as well. In some cases, a dispenser is provided for dispensing a liquid or solid formulation of the composition described herein. For example, a transfer pipette can be used to drop liquid onto a skin surface or onto eyes for intraocular delivery. In other cases, a dispenser can be utilized for dispensing capsules which can be required to be sterilely saved prior to application. Sterility, humidity, and/or temperature can be maintained, if required, in the containers as described herein. In some cases, an inhaler as discussed above can be a container for storing the composition in its liquid, solid, or aerosol form if needed. In certain embodiments, a container and an application tool can be provided separately.

A kit provided herein can comprise a first application tool, a second application tool, a sterilizing agent, and a composition free of any carrier comprising the polyribonucleotide and diluent, wherein the first application tool is configured to apply a sterilizing agent to a surface area of a subject and the second application tool is configured to apply the composition to the surface area of the subject. The sterilizing agent can be an alcohol, iodine, hydrogen peroxide, UV light, laser light, or heat. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. The first application tool can be a wipe or swab, wherein the wipe or swab comprises the sterilizing agent. Alternatively, the first application tool can be a device that applies UV light or laser light, or a device that applies heat. The second application tool can comprise a pipette. In some embodiments, the second application tool comprises a substrate, and wherein the substrate is embedded with the mixture. Often, the substrate is made of natural or artificial fibers. In some embodiments, the second application tool comprises a patch, a sprayer, or a nebulizer. In some cases, the second application tool is configured to release the mixture in a controlled manner. The surface area for application can be selected from the group consisting of: skin, surface areas of oral cavity, nasal cavity, gastrointestinal tract, and respiratory tract, and any combination thereof.

### Methods of Production

In some cases, the polyribonucleotide in the composition or pharmaceutical composition provided herein comprises a deoxyribonucleic acid sequence that is non-naturally occurring and can be produced using recombinant DNA technology (methods described in detail below; e.g., derived in vitro using a DNA plasmid) or chemical synthesis. The polyribonucleotide can be present in either linear or circular form.

It is within the scope of the invention that a DNA molecule used to produce an RNA circle can include a DNA sequence of a naturally-occurring original nucleic acid sequence, a modified version thereof, or a DNA sequence encoding a synthetic polypeptide not normally found in nature (e.g., chimeric molecules or fusion proteins). DNA molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The polyribonucleotide can be prepared according to any available technique including, but not limited to chemical synthesis and enzymatic synthesis. In some cases, a linear polyribonucleotide can be synthesized from ribonucleotide or transcribed from a DNA construct. The transcription from DNA construct can take place inside a cell or in vitro, using techniques available to one skilled in the art.

In some cases, a linear primary construct or linear mRNA can be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

All references and publications cited herein are hereby incorporated by reference.

### EMBODIMENTS

In some aspects, a composition of the present disclosure comprises a mixture of a polyribonucleotide and a cell-penetrating agent, wherein the cell-penetrating agent constitutes at least about 0.3% v/v of the mixture.

In some aspects, a therapeutic composition of the present disclosure comprises a polyribonucleotide and a cell-penetrating agent, wherein the cell-penetrating agent is configured for topical administration.

In some aspects, a therapeutic composition of the present disclosure comprises a polyribonucleotide and a cell-penetrating agent, wherein the polyribonucleotide comprises a payload or a sequence encoding a payload and wherein the payload has a biological effect on a cell.

In some aspects, a therapeutic composition of the present disclosure comprises a polyribonucleotide and a cell-penetrating agent, wherein the polyribonucleotide is in an amount effective to have a biological effect on a cell or tissue and wherein the cell-penetrating agent is in an amount effective to have a biological effect on a cell or tissue.

In some aspects, a therapeutic composition of the present disclosure comprises a polyribonucleotide, a cell-penetrating agent, and a topical delivery excipient, wherein the topical delivery excipient comprises a stabilizer. In some embodiments, the stabilizer comprises glucose (4.5 g/L).

In some aspects, a suppository or other lipid based formulation of the present disclosure comprises a polyribonucleotide and a cell-penetrating agent.

In some aspects, an inhalable composition of the present disclosure comprises a mixture of a polyribonucleotide, a cell-penetrating agent, and a propellant.

In some aspects, a therapeutic composition of the present disclosure comprises a biodegradable scaffold loaded with polyribonucleotide and a cell-penetrating agent.

In some aspects, a method of delivering a polyribonucleotide to a cell or tissue comprises contacting the cell or tissue to a mixture comprising the polyribonucleotide and a cell-penetrating agent, wherein the cell-penetrating agent constitutes at least about 0.3% v/v of the mixture.

In some aspects, a method of delivering a therapeutic composition to a cell or tissue comprises contacting the cell or tissue to the therapeutic composition comprising a polyribonucleotide and a cell-penetrating agent, wherein the cell-penetrating agent is configured for topical administration. In some embodiments, the cell-penetrating agent comprises an alcohol. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol comprises ethanol. In some embodiments, the cell-penetrating agent constitutes at least about 10%, at least about 20%, at least about 30%, at least about 40%, at least about 50%, at least about 60%, at least about 70%, at least about 80%, at least about 90%, at least about 95%, at least about 98%, or about 100% v/v of the mixture. In some embodiments, the cell-penetrating agent constitutes about 100% v/v of the mixture. In some embodiments, the method further comprises mixing the polyribonucleotide with the cell-penetrating agent. In some embodiments, the polyribonucleotide is in a solid form before the mixing. In some embodiments, the polyribonucleotide is lyophilized before the mixing In some embodiments, the polyribonucleotide is in a liquid form before the mixing. In some embodiments, the polyribonucleotide is dissolved in a solvent before the mixing. In some embodiments, the polyribonucleotide comprises a payload or a sequence encoding a payload and wherein the payload has a biological effect on a cell or a tissue. In some embodiments, the polyribonucleotide is in an amount effective to have a biological effect on a cell and the cell-penetrating agent is in an amount effective to have a biological effect on a cell or a tissue.

In some aspects, a method of in vivo delivery of a polyribonucleotide comprises applying a mixture comprising the polyribonucleotide and a cell-penetrating agent onto a surface area of a subject.

In some aspects, a method of topical delivery of a polyribonucleotide comprises applying a mixture comprising the polyribonucleotide and a cell-penetrating agent onto a surface area of a subject.

In some aspects, a method of delivering a therapeutic polyribonucleotide to a subject comprises topically contacting a mixture comprising the therapeutic polyribonucleotide and a cell-penetrating agent to an epithelial surface, endothelial surface, exposed tissue, or open wound.

In some aspects, a method of treatment comprises applying a mixture comprising a polyribonucleotide and a cell-penetrating agent to a surface area of a subject with a condition or disease. In some embodiments, the cell-penetrating agent comprises an alcohol. In some embodiments, the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose. In some embodiments, the alcohol comprises ethanol. In some embodiments, the delivery is systemic. In some embodiments, the delivery is localized. In some embodiments, the cell-penetrating agent constitutes at least about 1%, at least about 5%, at least about 10%, at least about 20%, at least about 30%, at least about 40%, at least about 50%, at least about 60%, at least about 70%, at least about 80%, at least about 90%, at least about 95%, at least about 98%, or about 100% v/v of the mixture. In some embodiments, the cell-penetrating agent constitutes about 10% v/v of the mixture. In some embodiments, the surface area is selected from the group consisting of: skin, surface areas of oral cavity, nasal cavity, ear cavity, gastrointestinal tract, respiratory tract, vaginal, cervical, inter uterine, urinary tract, and eye. In some embodiments, applying comprises depositing a drop of the mixture directly onto the surface area. In some embodiments, applying comprises wiping the surface area with a patch, a gel, or a film embedded with the mixture. In some embodiments, applying comprises spraying the mixture onto the surface area. In some embodiments, applying comprises administering the mixture to the subject via aerosolization. In some embodiments, applying comprises administering the mixture to the subject via a suppository. In some embodiments, applying comprises administering the mixture to the subject via oral ingestion of a capsule containing the mixture, and wherein the capsule is configured to release the mixture inside gastrointestinal tract of the subject. In some embodiments, the cell comprises an epithelial cell. In some embodiments, the cell comprises a blood cell. In some embodiments, the polyribonucleotide comprises a linear polyribonucleotide. In some embodiments, the polyribonucleotide comprises a circular polyribonucleotide. In some embodiments, the circular polyribonucleotide has a translation efficiency at least 5%, at least 10%, at least 15%, at least 20%, at least 30%, at least 40%, at least 50%, at least 60%, at least 70%, at least 80%, at least 90%, at least 100%, at least 150%, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold greater than a linear counterpart. In some embodiments, the circular polyribonucleotide has a translation efficiency at least 5 fold greater than a linear. In some embodiments, the polyribonucleotide has a short term biological effect. In some embodiments, the polyribonucleotide is a linear polyribonucleotide. In some embodiments, the polyribonucleotide has a long term biological effect. In some embodiments, the polyribonucleotide is a circular polyribonucleotide. In some embodiments, a concentration of the polyribonucleotide in the mixture is at least about 50 ng/mL, at least about 100 ng/mL, at least about 500 ng/mL, at least about 1 μg/mL, at least about 2 μg/mL, at least about 3 μg/mL, at least about 4 μg/mL, at least about 5 μg/mL, at least about 10 μg/mL, at least about 20 μg/mL, at least about 50 μg/mL, at least about 100 μg/mL, at least about 200 μg/mL, at least about 500 μg/mL, at least about 1 mg/mL, at least about 2 mg/mL, at least about 5 mg/mL, at least about 10 mg/mL, at least about 20 mg/mL, at least about 50 mg/mL, or at least about 100 mg/mL.

In some aspects, a kit of the present disclosure comprises an application tool and a mixture comprising a polyribonucleotide and a cell-penetrating agent, wherein the application tool is configured to apply the mixture to a surface area of a subject. In some embodiments, the application tool comprises a pipette. In some embodiments, the application tool comprises a substrate, and wherein the substrate is embedded with the mixture. In some embodiments, the substrate is made of natural or artificial fibers. In some embodiments, the kit comprises a suppository. In some embodiments, the application tool comprises a patch. In some embodiments, the application tool comprises a sprayer. In some embodiments, the application tool comprises a nebulizer. In some embodiments, the application tool comprises a capsule configured to release the mixture inside gastrointestinal tract of the subject. In some embodiments, the application tool is configured to release the mixture in a controlled manner. In some embodiments, the surface area is selected from the group consisting of: skin, surface areas of oral cavity, nasal cavity, gastrointestinal tract, and respiratory tract, and any combination thereof.

### NUMBERED EMBODIMENTS

[1] A pharmaceutical composition comprising a mixture of a polyribonucleotide and ethanol, wherein the ethanol constitutes at least about 0.3% v/v to about 75% v/v of the mixture.

[2] The pharmaceutical composition of paragraph [1], wherein the ethanol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture.

[3] The pharmaceutical composition of paragraph [1], wherein the ethanol constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

[4] A pharmaceutical composition comprising a mixture of a polyribonucleotide and an alcohol, wherein the alcohol constitutes at least about 0.3% v/v to about 75% v/v of the mixture.

[5] The pharmaceutical composition of paragraph [4], wherein the alcohol constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture.

[6] The pharmaceutical composition of paragraph [4], wherein the alcohol constitutes at least about at 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

[7] The pharmaceutical composition of any one of paragraphs [4]-[6], wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[8] A pharmaceutical composition comprising a mixture of a polyribonucleotide and a cell-penetrating agent, wherein the cell-penetrating agent constitutes at least about 0.3% v/v to about 75% v/v of the mixture.

[9] The pharmaceutical composition of paragraph [8], wherein the cell-penetrating agent constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture.

[10] The pharmaceutical composition of paragraph [8], wherein the cell-penetrating agent constitutes at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

[11] The pharmaceutical composition any one of paragraphs [8]-[10], wherein the cell-penetrating agent is an alcohol.

[12] The pharmaceutical composition of paragraph [11], wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[13] The pharmaceutical composition of any one of paragraphs [1]-[12], wherein the polyribonucleotide encodes a protein.

[14] The pharmaceutical composition of paragraph [13], wherein the protein is a therapeutic protein.

[15] The pharmaceutical composition of paragraph [13] or [14], wherein the protein is a wound healing protein.

[16] The pharmaceutical composition of paragraph [15], wherein the wound healing protein is a growth factor.

[17] The pharmaceutical composition of paragraph [16], wherein the growth factor is EGF, PDGF, TGFβ, or VEGF.

[18] The pharmaceutical composition of any one of paragraphs [1]-[17], wherein the pharmaceutical composition is a liquid, gel, lotion, paste, cream, foam, or stick.

[19] The pharmaceutical composition of any one of paragraphs [1]-[18], wherein the polyribonucleotide is a linear polyribonucleotide.

[20] The pharmaceutical composition of any one of paragraphs [1]-[19], wherein the polyribonucleotide is an mRNA.

[21] The pharmaceutical composition of any one of paragraphs [1]-[20], wherein the polyribonucleotide lacks a cap or poly-A tail.

[22] The pharmaceutical composition of any one of paragraphs [1]-[18], wherein the polyribonucleotide is a circular polyribonucleotide.

[23] The pharmaceutical composition of any one of paragraphs [1]-[22], wherein the polyribonucleotide comprises a modified ribonucleotide.

[24] The pharmaceutical composition of any one of paragraphs [1]-[23], wherein the pharmaceutical composition has a pH of about 7.

[25] The pharmaceutical composition of any one of paragraphs [1]-[24], wherein the pharmaceutical composition has a viscosity that is about the same as water.

[26] The pharmaceutical composition of any one of paragraphs [1]-[25], wherein the pharmaceutical composition is substantially free of hydrophobic or lipophilic groups.

[27] The pharmaceutical composition of any one of paragraphs [1]-[26], wherein the pharmaceutical composition is substantially free of hydrocarbons.

[28] The pharmaceutical composition of any one of paragraphs [1]-[27], wherein the pharmaceutical composition is substantially free of cationic liposomes.

[29] The pharmaceutical composition of any one of paragraphs [1]-[28], wherein the pharmaceutical composition is substantially free of fatty acids, lipids, liposomes, cholesterol, or any combination thereof.

[30] The pharmaceutical composition of any one of paragraphs [4]-[29], wherein the cell penetrating agent is soluble in polar solvents.

[31] The pharmaceutical composition of any one of paragraphs [4]-[30], wherein the cell penetrating agent is insoluble in polar solvents.

[32] A therapeutic composition comprising a polyribonucleotide and an alcohol, wherein the alcohol is configured for topical administration.

[33] A therapeutic composition comprising a polyribonucleotide and an alcohol, wherein the polyribonucleotide comprises a payload or a sequence encoding a payload and wherein the payload has a biological effect on a cell.

[34] A therapeutic composition comprising a polyribonucleotide and an alcohol, wherein the polyribonucleotide is in an amount effective to have a biological effect on a cell or tissue and wherein the alcohol is in an amount effective to have a biological effect on a cell or tissue.

[35] A therapeutic composition comprising a polyribonucleotide, an alcohol, and a topical delivery excipient, wherein the topical delivery excipient comprises a stabilizer.

[36] A suppository or other lipid based formulation comprising a polyribonucleotide and an alcohol.

[37] An inhalable composition comprising a mixture of a polyribonucleotide, an alcohol, and a propellant.

[38] A therapeutic composition comprising a biodegradable scaffold loaded with polyribonucleotide and an alcohol.

[39] A therapeutic composition comprising a polyribonucleotide and a cell-penetrating agent, wherein the cell-penetrating agent is configured for topical administration.

[40] A therapeutic composition comprising a polyribonucleotide and a cell-penetrating agent, wherein the polyribonucleotide comprises a payload or a sequence encoding a payload and wherein the payload has a biological effect on a cell.

[41] A therapeutic composition comprising a polyribonucleotide and a cell-penetrating agent, wherein the polyribonucleotide is in an amount effective to have a biological effect on a cell or tissue and wherein the alcohol is in an amount effective to have a biological effect on a cell or tissue.

[42] A therapeutic composition comprising a polyribonucleotide, a cell-penetrating agent, and a topical delivery excipient, wherein the topical delivery excipient comprises a stabilizer.

[43] A suppository or other lipid based formulation comprising a polyribonucleotide and a cell-penetrating agent.

[44] An inhalable composition comprising a mixture of a polyribonucleotide, a cell-penetrating agent, and a propellant.

[45] A therapeutic composition comprising a biodegradable scaffold loaded with polyribonucleotide and a cell-penetrating agent.

[46] The therapeutic composition of paragraph [35] or paragraph [42], wherein the stabilizer comprises glucose (4.5 g/L).

[47] The therapeutic composition, suppository, other lipid base formulation, or inhalable composition of any one of paragraphs [39]-[38], wherein the cell-penetrating agent comprises an alcohol.

[48] The therapeutic composition, suppository, other lipid base formulation, or inhalable composition of any one of paragraphs [32]-[38] or [47], wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[49] The therapeutic composition, suppository, other lipid base formulation, or inhalable composition of paragraph [48], wherein the alcohol is ethanol.

[50] A method of delivering a polyribonucleotide to a subject comprising

a) applying a sterilizing agent to a surface area of the subject;

b) applying a composition free of any carrier comprising the polyribonucleotide and diluent to the surface area.

[51] The method of paragraph [50], wherein the sterilizing agent is an alcohol, UV light, laser light, or heat.

[52] A method of delivering a polyribonucleotide to a subject comprising

a) applying an alcohol to a surface area of the subject;

b) applying a composition free of any carrier comprising the polyribonucleotide and diluent to the surface.

[53] A method of delivering a polyribonucleotide to an epithelial cell comprising applying a composition free of any carrier comprising a diluent and a polyribonucleotide that is not modified to the epithelial cell.

[54] A method of delivering a polyribonucleotide to a subject comprising topically applying a composition comprising a mixture of a polyribonucleotide and ethanol to a surface area of the subject, wherein the ethanol constitutes at least about 0.3% v/v to about 75% v/v of the mixture.

[55] A method of delivering a polyribonucleotide to a subject comprising topically applying a composition comprising a mixture of a polyribonucleotide and an alcohol to a surface area of the subject, wherein the alcohol constitutes at least about 0.3% v/v to about 75% v/v of the mixture.

[56] A method of delivering a polyribonucleotide to a subject comprising topically applying a composition comprising a mixture of a polyribonucleotide and a cell-penetrating agent to a surface area of the subject, wherein the cell-penetrating agent constitutes at least about 0.3% v/v to about 75% v/v of the mixture.

[57] The method of any one of paragraphs [53]-[56], wherein the composition delivers the polyribonucleotide to a dermal or epidermal tissue of the subject.

[58] The method of paragraph [58], wherein the composition delivers the polyribonucleotide to the dermal or epidermal tissue of the subject without iontophoresis.

[59] A method of delivering a polyribonucleotide to a cell or tissue comprising contacting the cell or tissue to a mixture comprising the polyribonucleotide and an alcohol, wherein the cell-penetrating agent or alcohol constitutes at least about 0.3% v/v to about 75% v/v of the mixture.

[60] A method of delivering a therapeutic composition to a cell or tissue comprising contacting the cell or tissue to the therapeutic composition comprising a polyribonucleotide and an alcohol, wherein the cell-penetrating agent or alcohol is configured for topical administration.

[61] A method of in vivo delivery of a polyribonucleotide comprising applying a mixture comprising the polyribonucleotide and an alcohol onto a surface area of a subject.

[62] A method of delivering a polyribonucleotide to a subject comprising applying a mixture comprising the polyribonucleotide and an alcohol onto a surface area of a subject.

[63] A method of delivering a therapeutic polyribonucleotide to a subject comprising topically contacting a mixture comprising the therapeutic polyribonucleotide and an alcohol to an epithelial surface, endothelial surface, exposed tissue, or open wound.

[64] A method of treatment comprising applying a mixture comprising a polyribonucleotide and an alcohol to a surface area of a subject with a condition or disease.

[65] A method of delivering a polyribonucleotide to a cell or tissue comprising contacting the cell or tissue to a mixture comprising the polyribonucleotide and a cell-penetrating agent, wherein the cell-penetrating agent or alcohol constitutes at least about 0.3% v/v to about 75% v/v of the mixture.

[66] A method of delivering a therapeutic composition to a cell or tissue comprising contacting the cell or tissue to the therapeutic composition comprising a polyribonucleotide and a cell-penetrating agent, wherein the cell-penetrating agent or alcohol is configured for topical administration.

[67] A method of in vivo delivery of a polyribonucleotide comprising applying a mixture comprising the polyribonucleotide and a cell-penetrating agent onto a surface area of a subject.

[68] A method of delivering a polyribonucleotide to a subject comprising applying a mixture comprising the polyribonucleotide and a cell-penetrating agent onto a surface area of a subject.

[69] A method of delivering a therapeutic polyribonucleotide to a subject comprising topically contacting a mixture comprising the therapeutic polyribonucleotide and a cell-penetrating agent to an epithelial surface, endothelial surface, exposed tissue, or open wound.

[70] A method of treatment comprising applying a mixture comprising a polyribonucleotide and a cell-penetrating agent to a surface area of a subject with a condition or disease.

[71] A method of treating a wound comprising contacting the wound or tissue surrounding the wound to a composition comprising a mixture of a polyribonucleotide and ethanol, wherein the ethanol constitutes at least about 0.3% v/v to about 75% v/v of the mixture.

[72] A method of treating a wound comprising contacting the wound or tissue surrounding the wound to a composition comprising a mixture of a polyribonucleotide and ethanol, wherein the alcohol constitutes at least about 0.3% v/v to about 75% v/v of the mixture.

[73] A method of treating a wound comprising contacting the wound or tissue surrounding the wound to a composition comprising a mixture of a polyribonucleotide and ethanol, wherein the cell-penetrating agent constitutes at least about 0.3% v/v to about 75% v/v of the mixture.

[74] The method of any one of paragraphs [65]-[70] or [73], wherein the cell-penetrating agent comprises an alcohol.

[75] The method of any one of paragraphs [51]-[65] or [74], wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

[76] The method of paragraph [51]-[65], [74], or [75], wherein the alcohol comprises ethanol.

[77] The method of any one of paragraphs [59]-[76], wherein the ethanol, alcohol, or cell-penetrating agent constitutes at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture.

[78] The method of any one of paragraphs [59]-[76] wherein the ethanol, alcohol, or cell-penetrating agent at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

[79] The method of any one of paragraphs [65]-[78], further comprising mixing the polyribonucleotide with the cell-penetrating agent.

[80] The method of any one of paragraphs [59]-[64] or [71]-[78], further comprising mixing the polyribonucleotide with the alcohol.

[81] The method of paragraphs [79] or [80], wherein the polyribonucleotide is in a solid form before the mixing.

[82] The method of paragraph 78, wherein the polyribonucleotide is lyophilized before the mixing.

[83] The method of paragraphs [79] or [80], wherein the polyribonucleotide is in a liquid form before the mixing.

[84] The method of paragraphs [79] or [80], wherein the polyribonucleotide is dissolved in a solvent before the mixing.

[85] The method of any one of paragraphs [50]-[84], wherein the polyribonucleotide comprises a payload or a sequence encoding a payload and wherein the payload has a biological effect on a cell or a tissue.

[86] The method of any one of paragraphs [50]-[85], wherein the polyribonucleotide is in an amount effective to have a biological effect on a cell and the cell-penetrating agent is in an amount effective to have a biological effect on a cell or a tissue.

[87] The method of any one of paragraphs [50]-[86], wherein the polyribonucleotide encodes a protein.

[88] The method of paragraph [87], wherein the protein is a therapeutic protein.

[89] The method of paragraph [87] or [88], wherein the protein is a wound healing protein.

[90] The method of paragraph [89], wherein the wound healing protein is a growth factor.

[91] The method of paragraph [90], wherein the growth factor is EGF, PDGF, TGFβ, or VEGF.

[92] The method of any one of paragraphs [50]-[91], wherein the composition is a liquid, gel, lotion, paste, cream, foam, or stick.

[93] The method of any one of paragraphs [50]-[92], wherein the polyribonucleotide is a linear polyribonucleotide.

[94] The method of any one of paragraphs [50]-[93], wherein the polyribonucleotide is an mRNA.

[95] The method of any one of paragraphs [50]-[94], wherein the polyribonucleotide lacks a cap or poly-A tail.

[96] The method of any one of paragraphs [50]-[92], wherein the polyribonucleotide is a circular polyribonucleotide.

[97] The method of any one of paragraphs [50]-[96], wherein the polyribonucleotide comprises a modified ribonucleotide.

[98] The method of any one of paragraphs [50]-[97], wherein the composition has a pH of about 7.

[99] The method of any one of paragraphs [50]-[98], wherein the composition has a viscosity that is about the same as water.

The method of any one of paragraphs [50]-[99], wherein the composition is substantially free of hydrophobic or lipophilic groups.

The method of any one of paragraphs [50]-[100], wherein the composition is substantially free of hydrocarbons.

The method of any one of paragraphs [50]-[101], wherein the composition is substantially free of cationic liposomes.

The method of any one of paragraphs [50]-[102], wherein the composition is substantially free of fatty acids, lipids, liposomes, cholesterol, or any combination thereof.

The method of any one of paragraphs [59]-[103], wherein the cell penetrating agent is soluble in polar solvents.

The method of any one of paragraphs [59]-87, wherein the cell penetrating agent is insoluble in polar solvents.

The method of any one of paragraphs [50]-[105], wherein the composition further comprises a pharmaceutically acceptable excipient.

The method of any one of paragraphs [50]-[106], wherein the delivery is systemic.

The method of any one of paragraphs [50]-[106], wherein the delivery is localized.

The method of any one of paragraphs [50]-[108], wherein the surface area is selected from the group consisting of: skin, surface areas of oral cavity, nasal cavity, ear cavity, gastrointestinal tract, respiratory tract, vaginal, cervical, inter uterine, urinary tract, and eye.

The method of any one of paragraphs [50]-[58], [61], [62], or [71]-[109], wherein applying comprises depositing a drop of the mixture directly onto the surface area.

The method of any one of paragraphs [50]-[58], [61], [62], or [71]-[109], wherein applying comprises wiping the surface area with a patch, a gel, or a film embedded with the mixture.

The method of any one of paragraphs [50]-[58], [61], [62], or [71]-[109], wherein applying comprises spraying the mixture onto the surface area.

The method of any one of paragraphs [50]-[58], [61], [62], or [71]-[109], wherein applying comprises administering the mixture to the subject via aerosolization.

The method of any one of paragraphs [50]-[58], [61], [62], or [71]-[109], wherein applying comprises administering the mixture to the subject via a suppository.

The method of any one of paragraphs [50]-[58], [61], [62], or [71]-[109], wherein applying comprises administering the mixture to the subject via oral ingestion of a capsule containing the mixture, and wherein the capsule is configured to release the mixture inside gastrointestinal tract of the subject.

The method of any one of paragraphs [59], [60], [65], [66], or [71]-[115], wherein the cell comprises an epithelial cell.

The method of any one of paragraphs [96]-[116], wherein the circular polyribonucleotide has a translation efficiency at least 5%, at least 10%, at least 15%, at least 20%, at least 30%, at least 40%, at least 50%, at least 60%, at least 70%, at least 80%, at least 90%, at least 100%, at least 150%, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold greater than a linear counterpart.

The method of any one of paragraphs [96]-[117], wherein the circular polyribonucleotide has a translation efficiency at least 5 fold greater than a linear counterpart.

The method of any one of paragraphs [50]-[118], wherein the polyribonucleotide has a short term biological effect.

The method of any one of paragraphs [50]-[119], wherein the polyribonucleotide has a long term biological effect.

The method of any one of paragraphs [50]-[120], wherein a concentration of the polyribonucleotide in the mixture is at least about 50 ng/mL, at least about 100 ng/mL, at least about 500 ng/mL, at least about 1 μg/mL, at least about 2 μg/mL, at least about 3 μg/mL, at least about 4 μg/mL, at least about 5 μg/mL, at least about 10 μg/mL, at least about 20 μs/mL, at least about 50 μg/mL, at least about 100 μg/mL, at least about 200 μg/mL, at least about 500 μs/mL, at least about 1 mg/mL, at least about 2 mg/mL, at least about 5 mg/mL, at least about 10 mg/mL, at least about 20 mg/mL, at least about 50 mg/mL, or at least about 100 mg/mL.

A kit comprising an application tool and the pharmaceutical composition of any one of paragraphs [1]-[31], wherein the application tool is configured to apply the pharmaceutical composition to a surface area of a subject.

A kit comprising a first application tool, a second application tool, a sterilizing agent, and a composition free of any carrier comprising the polyribonucleotide and diluent, wherein the first application tool is configured to apply a sterilizing agent to a surface area of a subject and the second application tool is configured to apply the composition to the surface area of the subject.

The kit of paragraph [123], wherein the sterilizing agent is an alcohol, UV light, laser light, or heat.

The kit of paragraph [124], wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

The kit of any one of paragraphs [123]-[125], wherein the first application tool is a wipe.

The kit of paragraph [126], wherein the wipe comprises the sterilizing agent.

The kit of paragraph [123] or [124], wherein first application tool is a device that applies UV light or laser light.

The kit of paragraph [123] or [124], wherein the first application tool is a device that applies heat.

A kit comprising an application tool and a mixture comprising a polyribonucleotide and a cell-penetrating agent, wherein the application tool is configured to apply the mixture to a surface area of a subject.

The kit of any one of paragraphs [122]-[130], wherein the application tool or second application tool comprises a pipette.

The kit of any one of paragraphs [122]-[130], wherein the application tool or second application tool comprises a substrate, and wherein the substrate is embedded with the mixture.

The kit of paragraph [132], wherein the substrate is made of natural or artificial fibers.

The kit of paragraph [132], wherein the kit comprises a suppository.

The kit of any one of paragraphs [122]-[134], wherein the application tool or second application tool comprises a patch.

The kit of any one of paragraphs [122]-[134], wherein the application tool or second application tool comprises a sprayer.

The kit of any one of paragraphs [122]-[134], wherein the application tool or second application tool comprises a nebulizer.

The kit of any one of paragraphs [122]-[134], wherein the application tool or second application tool comprises a capsule configured to release the mixture inside gastrointestinal tract of the subject.

The kit of any one of paragraphs [122]-[138], wherein the application tool or second application tool is configured to release the mixture in a controlled manner.

The kit of any one of paragraphs [122]-[139], wherein the surface area is selected from the group consisting of: skin, surface areas of oral cavity, nasal cavity, gastrointestinal tract, and respiratory tract, and any combination thereof.

A method of treating a wound, comprising contacting the wound or tissue surrounding the wound to a composition comprising any one of paragraphs [1]-[31].

### EXAMPLES

The following examples are provided to further illustrate some embodiments of the present invention, but are not intended to limit the scope of the invention; it will be understood by their exemplary nature that other procedures, methodologies, or techniques known to those skilled in the art can alternatively be used.

### Example 1: Formulation of RNA for Topical Delivery

This Example demonstrates formulation of RNA for topical delivery.

To determine topical effects of RNA, RNA was formulated for delivery to epithelial tissues.

As described herein, RNA was formulated with a cell-penetrating agent according to the following:

10 ng RNA linear or circular comprising an EGF ORF

5 uL 80% ethanol

35 uL PBS+glucose (4.5 g/L)

### Example 2: Topical Delivery of Linear RNA

This Example demonstrates topical delivery of RNA.

To determine topical delivery effects, RNA was formulated and delivered to epithelial tissues. As described herein, linear RNA formulated with a cell-penetrating agent was delivered topically to ear tissue.

Samples of linear RNA were formulated as in Example 1 (50 μL) and applied to an ear of a mouse. Ears were wiped with an isopropyl alcohol wipe prior to application of the samples to the ears. Samples were dried by exposing the ears briefly to a heatlamp and fan in a sterilized hood. Mice were placed back in cages under normal conditions.

At select timepoints (6 hrs, 1 day, 3 days, or 12 days post application), ear tissues (through a single ear punch) were collected for each RNA sample and stored in a tissue storage reagent (e.g., permeates the tissue to stabilize and protect cellular RNA in unfrozen samples).

### Example 3: Topical Delivery of Circular RNA

This Example demonstrates topical delivery of RNA.

To determine topical delivery effects, RNA was formulated and delivered to epithelial tissues. As described herein, RNA was formulated with a cell-penetrating agent and delivered topically to ear tissue.

Samples of circular RNA were formulated as in Example 1 (50 μL) and applied to both ears of a mouse. Ears were wiped with an isopropyl alcohol wipe prior to application of the samples to the ears. Samples were dried on the ears by exposing the ears briefly to a heatlamp and fan in a sterilized hood. Mice were placed back in cages under normal conditions.

At select timepoints (6 hrs, 1 day, 3 days or 12 days post application), ear tissues (through a single ear punch) were collected for each RNA sample.

### Example 4: Persistence of RNA after Topical Delivery

This Example demonstrates RNA presence after topical delivery.

To determine RNA persistence, tissue samples were analyzed for delivered RNA. As described herein, ear punches were analyzed for persistence at varying timepoints after topical delivery of the RNA.

Ear punch samples from Example 3 and untreated ear punch samples were collected in an RNA stabilization reagent and RNA was extracted using a standard RNA tissue extraction kit (Maxwell RSC simply RNA).

A volume of 200 μl of 1-Thioglycerol/Homogenization Solution was added to each sample. A working solution was prepared by adding 20 μl of 1-Thioglycerol per milliliter of Homogenization Solution. Alternatively, 600 μl of 1-Thioglycerol was added to the 30 ml bottle of Homogenization Solution. Before use, the 1-Thioglycerol/Homogenization Solution was chilled on ice or at 2-10° C.

The tissue samples were homogenized in 200 μl of chilled 1-Thioglycerol/Homogenization Solution with a handheld homogenizer and sterile pestle until no visible tissue fragments remained. Each sample was homogenized an additional 15-30 seconds for complete homogenization.

To check for the presence of RNA at the different timepoints, the samples were checked for RNA via q-PCR. qPCR was used to measure the presence of both linear and circular RNA in the ear punches. To detect linear and circular RNA, primers that amplified the Nluc ORF were used. (F: AGATTTCGTTGGGGACTGGC (SEQ ID NO: 7), R: CACCGCTCAGGACAATCCTT (SEQ ID NO: 8)). To detect only circular RNA, primers that amplified the 5′-3′ junction allowed for detection of circular but not linear RNA constructs (F: CTGGAGACGTGGAGGAGAAC (SEQ ID NO: 9), R: CCAAAAGACGGCAATATGGT (SEQ ID NO: 10)).

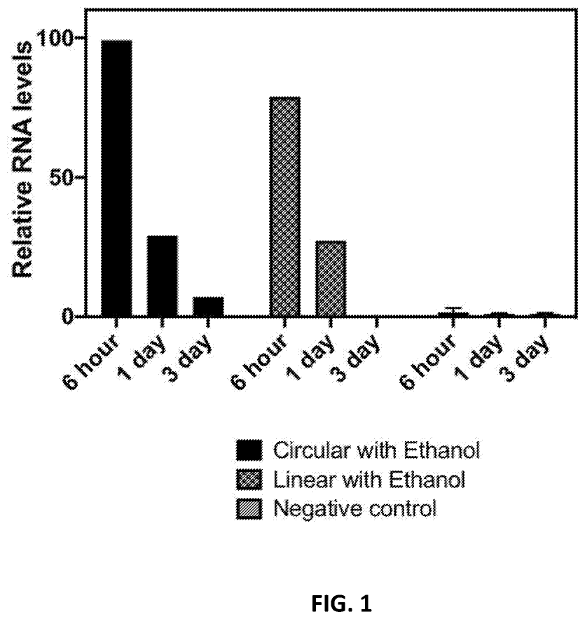

Linear and circular RNA was detected at 6 hrs, 24 hrs, and 72 hrs after topical delivery. Higher levels of circular RNA compared to linear RNA were detected in ears of mice at 3-days post-injection (FIG. 1).

As shown in this Example, linear and circular RNA administered topically were detectable in vivo.

### Example 5: Protein Expression of mRNA after Topical Delivery

This Example describes protein presence after topical delivery.

To determine if topical delivered RNA can be translated, tissue samples are analyzed for protein expression at the different timepoints by western blot. Ear punches are analyzed for protein expression after topical delivery of the RNA.

In short, the ear punches are collected and stored in an RNA stabilization reagent (Invitrogen). The tissue is homogenized in RIPA buffer with micro tube homogenizer (Fisher scientific) and protein is extracted. Each sample is centrifuged at 14k×g for 15 mins.

The supernatant is removed and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min.

A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

It is expected that the GFP protein is visualized in ear punch samples and is detected in circular RNA and linear RNA.

### Example 6: Topical Administration of RNA Results in RNA Delivery to Tissue when Ethanol is Included in the RNA Solution

This Example demonstrates the ability to deliver RNA to cells and tissues via topical administration in vivo when ethanol is included in the RNA solution.

In this example, circular RNA was designed with an EMCV IRES and ORF encoding Nanoluciferase (NLuc).

The circular RNA was generated in vitro. Unmodified linear RNA was transcribed in vitro from a DNA template. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA circularized using a splint DNA and T4 RNA ligase 2 (New England Biolabs, M0239). Circular RNA was then Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000). In this example, circular RNA was also HPLC-purified.

In this example, linear mRNA was designed with an ORF encoding a Nano Luciferase (NLuc). In this example, modified linear mRNA was made in-house by in vitro transcription. In this example, linear RNA was fully substituted with Pseudo-Uridine and 5-Methyl-C, capped with CleanCap™ AG, included 5′ and 3′ human alpha-globin UTRs, and is polyadenylated.

RNA was then diluted in PBS/Glucose (4.5 g/L) and ethanol (10% v/v) such that total sample volume for each sample was 50 uL, and total RNA for each sample was 3.5 pmoles. All reagents are brought to room temperature prior to mixing and mixtures are prepared immediately prior to use.

At time=0, 50 μL dose of each sample was applied topically to the ear of a BALB/c mouse dropwise using a pipet tip. As a negative control, an untreated mouse was used. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 6 hours, 1, 3, and 12 days post-administration, a 2 mm ear punch was taken from each animal and stored in RNAlater solution (ThermoFisher Scientific, cat #AM7020). Total RNA was isolated from ear punches using trizol extraction. The aqueous-phase was precipitated with isopropanol and the pellet was washed with 70% ETOH as per manufacturer's instructions. cDNA was synthesized from the total RNA and RT-PCR was performed on cDNA templates using primers specific to the NLuc ORF. All samples were assayed in triplicate on the Bio-rad CFX384 Thermal Cycler. RNA levels were then relativized to actin and to the untreated negative control.

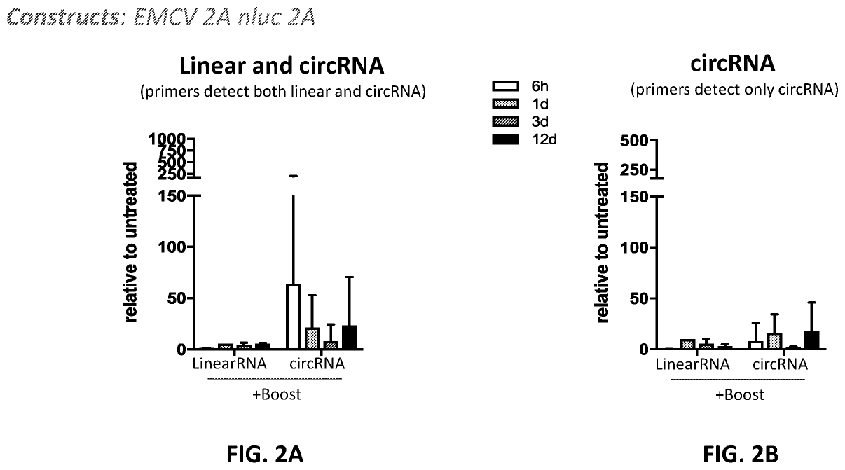

Circular RNA and linear RNA were detected in tissue samples at 6 hours and 1, 3 and 12 days after topical administration and showed greater signal than the vehicle only control (FIG. 2A & FIG. 2B).

This Example demonstrates that circular RNA and linear RNA are successfully delivered via topical administration to the tissue when delivered with ethanol and persists in tissue over prolonged periods of time.

### Example 7: Topical Administration of RNA Results in RNA Delivery to Tissue when Formulated with TransIT

This Example demonstrates the ability to deliver RNA to cells and tissues via topical administration in vivo when TransIT is used to formulate the RNA solution

In this example, circular RNA was designed with an EMCV IRES and ORF encoding Nanoluciferase (NLuc).

The circular RNA was generated in vitro. Unmodified linear RNA was transcribed in vitro from a DNA template. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA circularized using a splint DNA and T4 RNA ligase 2 (New England Biolabs, M0239). Circular RNA was then Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000). In this example, circular RNA was also HPLC-purified.

In this example, linear mRNA was designed with an ORF encoding a Nano Luciferase (NLuc). In this example, modified linear mRNA was made in-house by in vitro transcription. In this example, linear RNA was fully substituted with Pseudo-Uridine and 5-Methyl-C, capped with CleanCap™ AG, included 5′ and 3′ human alpha-globin UTRs, and is polyadenylated.

RNA was diluted in PBS/Glucose (4.5 g/L) to afford 3.5 pmoles in 10 uL of solution. This RNA solution was then added to TransIT (Minis Bio, MIR5700) (10 uL), Boost (Minis Bio, MIR5700) (5 uL), and PBS/Glucose (4.5 g/L) (25 uL). The total sample volume for each sample was 50 uL, and total RNA for each sample was 3.5 pmoles. All reagents are brought to room temperature prior to mixing and mixtures are prepared immediately prior to use.

At time=0, 50 μL dose of each sample was applied topically to the ear of a BALB/c mouse dropwise using a pipet tip. As a negative control, an untreated mouse was used. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 6 hours, 1, 3, and 12 days post-administration, a 2 mm ear punch was taken from each animal and stored in RNAlater solution (ThermoFisher Scientific, cat #AM7020). Total RNA was isolated from ear punches using trizol extraction. The aqueous-phase was precipitated with isopropanol and the pellet was washed with 70% ETOH as per manufacturer's instructions. cDNA was synthesized from the total RNA and RT-PCR was performed on cDNA templates using primers specific to the NLuc ORF. All samples were assayed in triplicate on the Bio-rad CFX384 Thermal Cycler. RNA levels were then relativized to actin and the untreated negative control.

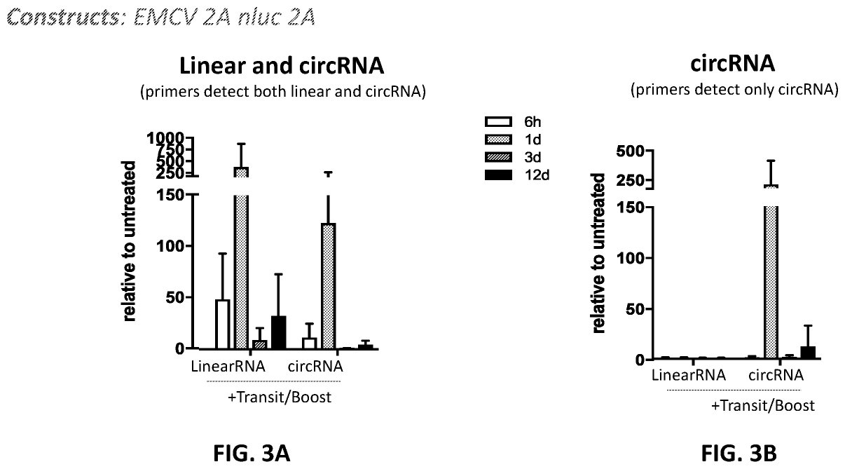

Circular RNA and linear RNA were detected in tissue samples at 6 hours, 1, 3 and 12 days after topical administration and showed greater signal than the vehicle only control (FIG. 3A and FIG. 3B).

This Example demonstrates that circular RNA and linear RNA are successfully delivered via topical administration to the tissue when delivered with TransIT and persists in tissue over prolonged periods of time.

### Example 8: Topical Administration of Modified Linear RNA Formulated with Dimethyl Sulfoxide (DMSO) Gel In Vivo

This Example demonstrates the ability to deliver linear RNA in vivo by topical administration when formulated with DMSO gel.

For this Example, RNAs included an ORF encoding Gaussia Luciferase (GLuc).

In this example, modified linear RNA was custom synthesized by Trilink Biotechnologies and included all the motifs listed above. In this example, RNA was fully substituted with Pseudo-Uridine and 5-Methyl-C, capped with CleanCap™ AG and is polyadenylated (120A). DMSO Medi Gel (21^{st} Century Chemical Inc.) was commercially available.

RNA was diluted to a concentration of 1 pmole/μL in RNA storage solution. 5 pmole of RNA was combined with 19 μL of DMSO Medi Gel (21^{st} Century Chemical Inc.) and 1 μL of Rnasin Plus RNase Inhibitor (Promega) for a total of 25 μL per application. Formulation without RNA was used as a control.

At time=0, a 25 μL dose of each sample was applied topically to the ear of a mouse using a pipet tip. Samples were dried by exposing the ears briefly to a heat lamp and fan in a sterilized hood. Mice were place back in cages under normal conditions.

To determine RNA expression in tissue, tissue samples were analyzed for RNA expression at varying timepoints after topical delivery. Ear punches were taken from the mouse at 24 and 48 hours after delivery. Tissue samples were placed in 1× Luciferase Cell Lysis Buffer (Thermo Scientific) on ice for 30 minutes and then frozen.

The activity of Gaussia Luciferase was tested using a Gaussia Luciferase Activity assay (Thermo Scientific Pierce). Samples were thawed and spun briefly to remove any tissue debris. 20 μL of the buffer solution was added to a 96 well plate (Corning 3990). In brief, 1× coelenterazine substrate was added to each well. Plates were read immediately after substrate addition and mixing in a luminometer instrument (Promega).

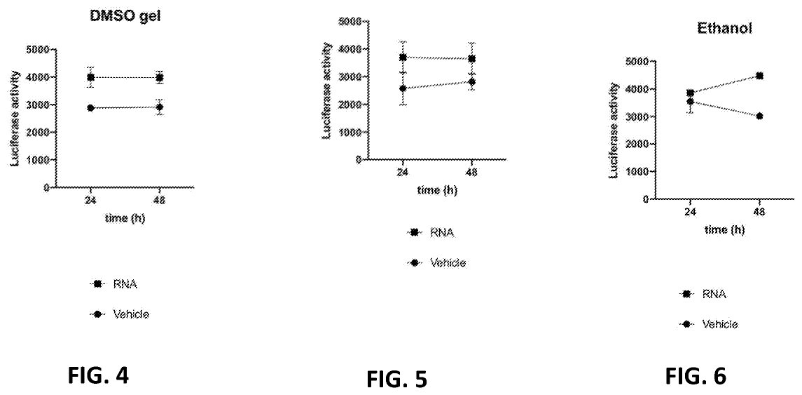

Gaussia Luciferase activity was detected in tissue samples at 24 and 48 hours after topical application and was observed to be higher than the vehicle only control (FIG. 4).

This Example demonstrated that linear RNA was successfully delivered via topical administration when formulated with DMSO Medi Gel (21^{st} Century Chemical Inc.) and was able to express functional protein detectable in tissue for prolonged periods of time.

### Example 9: Topical Administration of Modified Linear RNA Formulated with Cream-Based Ointment In Vivo

This Example demonstrates the ability to deliver linear RNA in vivo by topical administration when formulated with the cream-based ointment, Johnson&Johnson's baby lotion.

For this Example, RNAs included an ORF encoding Gaussia Luciferase (GLuc).

In this example, modified linear RNA was custom synthesized by Trilink Biotechnologies and included all the motifs listed above. In this example, RNA was fully substituted with Pseudo-Uridine and 5-Methyl-C, capped with CleanCap™ AG and is polyadenylated (120A). Baby lotion (Johnson&Johnson) was available commercially.

RNA was diluted to a concentration of 1 pmole/μL. 5 pmole of RNA was combined with 19 μL of Johnson's baby lotion (no fragrance; Johnson & Johnson) and 1 μL of Rnasin Plus RNase Inhibitor (Promega) for a total of 25 μL per application. Formulation without RNA was used as a control.

At time=0, a 25 μL dose of each sample was applied topically to the ear of a mouse using a pipet tip. Samples were dried by exposing the ears briefly to a heat lamp and fan in a sterilized hood. Mice were place back in cages under normal conditions.

To determine RNA expression in tissue, tissue samples were analyzed for RNA expression at varying timepoints after topical delivery. Ear punches were taken from the mouse at 24 and 48 hours after delivery. Tissue samples were placed in 1× Luciferase Cell Lysis Buffer (Thermo Scientific) on ice for 30 minutes and then frozen.

The activity of Gaussia Luciferase was tested using a Gaussia Luciferase Activity assay (Thermo Scientific Pierce). Samples were thawed and spun briefly to remove any tissue debris. 20 μL of the buffer solution was added to a 96 well plate (Corning 3990). In brief, 1× coelenterazine substrate was added to each well. Plates were read immediately after substrate addition and mixing in a luminometer instrument (Promega).

Gaussia Luciferase activity was detected in tissue samples at 24 and 48 hours after topical application and was observed to be higher than the vehicle only control (FIG. 5).

This Example demonstrated that linear RNA was successfully delivered via topical administration when formulated with a cream-based ointment and was able to express functional protein detectable in tissue for prolonged periods of time.

### Example 10: Topical Administration of Modified Linear RNA Using Ethanol In Vivo

This Example demonstrates the ability to deliver linear RNA in vivo by topical administration using ethanol.

For this Example, RNAs included an ORF encoding Gaussia Luciferase (GLuc).

In this example, modified linear RNA was custom synthesized by Trilink Biotechnologies and included all the motifs listed above. In this example, RNA was fully substituted with Pseudo-Uridine and 5-Methyl-C, capped with CleanCap™ AG and is polyadenylated (120A). Ethanol (Sigma Aldrich) was available commercially.

RNA was diluted to a concentration of 1 pmole/μL with RNA storage solution. 5 pmole of RNA was combined with 19 μL of ethanol and 1 μL of Rnasin Plus RNase Inhibitor (Promega) for a total of 25 μL per application. Vehicle only control was similarly prepared but did not contain RNA.

At time=0, a 25 μL dose of each sample was applied topically to the ear of a mouse using a pipet tip. Samples were dried by exposing the ears briefly to a heat lamp and fan in a sterilized hood. Mice were place back in cages under normal conditions.

To determine RNA expression in tissue, tissue samples were analyzed for RNA expression at varying timepoints after topical delivery. Ear punches were taken from the mouse at 24 and 48 hours after delivery. Tissue samples were placed in 1× Luciferase Cell Lysis Buffer (Thermo Scientific) on ice for 30 minutes and then frozen.

The activity of Gaussia Luciferase was tested using a Gaussia Luciferase Activity assay (Thermo Scientific Pierce). Samples were thawed and spun briefly to remove any tissue debris. 20 μL of the buffer solution was added to a 96 well plate (Corning 3990). In brief, 1× coelenterazine substrate was added to each well. Plates were read immediately after substrate addition and mixing in a luminometer instrument (Promega).

Gaussia Luciferase activity was detected in tissue samples at 24 and 48 hours after topical application and was observed to be higher than the vehicle only control (FIG. 6).

This Example demonstrated that linear RNA was successfully delivered via topical administration with ethanol and was able to express functional protein detectable in tissue for prolonged periods of time.

### Example 11: Topical Administration of Circular RNA Results in RNA Delivery to Tissue

This Example demonstrates the ability to delivery circular RNA to cells and tissues via topical administration in vivo.

In this example, circular RNA was designed with an ORF encoding an erythropoietin protein (EPO).

The circular RNA was generated in vitro. Linear RNA was transcribed in vitro from a DNA template including all the motifs listed above, as well as a T7 RNA polymerase promoter to drive transcription. In this example, Cy5-UTP is used to generate Cy5-labeled RNA. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

Circular RNA was diluted in PBS/glucose (4.5 g/L) with 5% ethanol such that total sample volume for each sample was 25 uL, and total RNA for each sample was 12 picomoles. All reagents were brought to room temperature prior to mixing and mixtures were prepared immediately prior to use.

At time=0, the ear of the mouse was wiped with an isopropyl alcohol wipe, dried and a 25 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine circular RNA delivery to tissue, tissue samples were analyzed by fluorescence microscopy at varying timepoints after administration. At 6 hours, 1 day, and 3 days post administration, a 2 mm ear punch was taken from each animal and stored in ice-cold PBS. Tissue samples were then observed under an EVOS II fluorescent microscope. Images were then quantified for fluorescence using Image.”

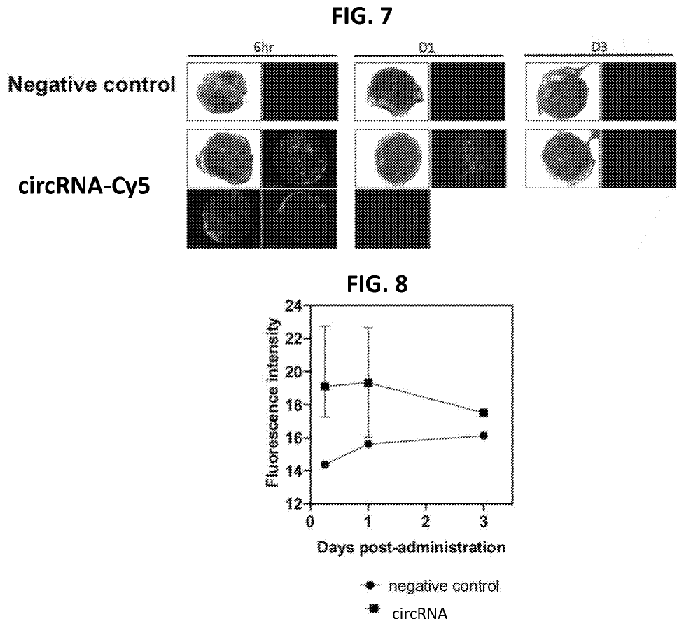

Cy5 signal was detected in tissue samples at 6 hours, 1 and 3 days after topical administration and showed greater signal than the negative control which did not show any fluorescence (FIG. 7 and FIG. 8). This indicates that circular RNA is successfully delivered to the tissue.

This Example demonstrates that circular RNA is successfully delivered via topical administration when the skin and persists in tissue over prolonged periods of time.

### Example 12: Topical Administration of mRNA Results in RNA Delivery to Tissue

This Example demonstrates the ability to delivery mRNA to cells and tissues via topical administration in vivo.

In this example, mRNA was designed with an ORF encoding a green fluorescent protein (eGFP). In this example, modified linear mRNA was custom synthesized by Trilink Biotechnologies and included all the motifs listed above. In this example, RNA was fully substituted with Pseudo-Uridine and 5-Methyl-C, capped with CleanCap™ AG, included 5′ and 3′ human alpha-globin UTRs, and is polyadenylated. mRNA included a Cy5 fluorophore label, covalently bound at the 3′ end.

mRNA was diluted in PBS/glucose (4.5 g/L) with 5% ethanol such that total sample volume for each sample was 25 uL, and total RNA for each sample was 12 picomoles. All reagents were brought to room temperature prior to mixing and mixtures were prepared immediately prior to use.

At time=0, the ear of the mouse was wiped with an isopropyl alcohol wipe, dried and a 25 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA delivery to tissue, tissue samples were analyzed by fluorescence microscopy at varying timepoints after administration. At 6 hours, 1 day, and 3 days post administration, a 2 mm ear punch was taken from each animal and stored in ice-cold PBS. Tissue samples were then observed under an EVOS II fluorescent microscope. Images were then quantified for fluorescence using ImageJ.

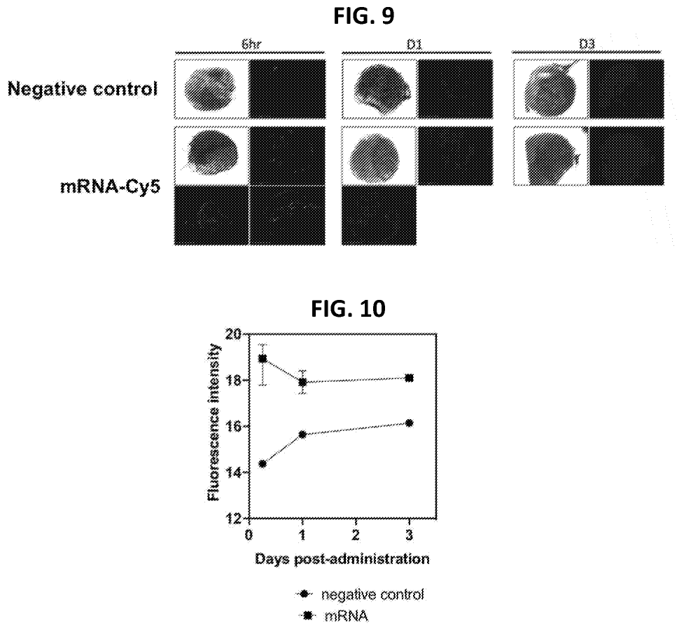

Cy5 signal was detected in tissue samples at 6 hours, 1 day, and 3 days after topical administration and showed greater signal than the negative control which did not show any fluorescence (FIG. 9 and FIG. 10). This indicates that mRNA is successfully delivered to the tissue.

This Example demonstrates that mRNA is successfully delivered via topical administration when the skin and persists in tissue over prolonged periods of time.

### Example 13: Topical Administration of Unmodified RNA to the Nasal Mucosal Epithelium Results in Persistence of RNA in Tissue

This Example describes the ability to delivery unmodified RNA via topical administration to the nasal mucosal epithelium and achieve uptake of linear and circular RNA via topical administration.

For this Example, an IRES, an ORF encoding Nano Luciferase (NLuc), and two spacer elements flanking the IRES-ORF are included in the RNA.

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA is circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA is Urea-PAGE purified, eluted in a buffer (0.5 M Sodium Acetate, 0.1% SDS, 1 mM EDTA), ethanol precipitated and resuspended in RNaA storage solution (ThermoFisher Scientific, cat #AM7000). Linear RNA counterparts are generated and included the same NLuc ORF and coding components described above.

RNA is diluted to a concentration of 1 pmole/uL. 5 pmole of RNA in citrate buffer is combined with sterile PBS. Total sample volume to be used per application is 20 uL Vehicle only control samples are prepared similarly but without RNA. All reagents are brought to room temperature prior to mixing and mixtures are prepared immediately prior to use.

In a sterilized hood, mice are placed in a hanging position by their ears. At time=0, a 20 uL dose of each sample is gradually release into the nostrils of a BALB/c mouse (10 uL in each nostril) using a micropipette. Mouth and alternate nostril are closed during application to ensure uptake. Mice are kept in the hanging position for an additional few minutes until breathing rate returned to normal. Mice are placed back in cages under normal conditions.

At 6, 24 and 48 hours post administration, mice are sacrificed and nasal tissue is taken from the mouse. Each tissue sample (˜2 mg) is placed in 200 uL of chilled 1-thioglycerol/Homogenization solution and homogenized using a handheld homogenized and sterile pestle until no visible tissue fragments remain. Each sample is homogenized for an additional 15-30 seconds for complete homogenization.

To determine RNA persistence in tissue, tissue samples are analyzed for RNA at varying timepoints after delivery using qPCR. qPCR is used to measure both linear and circular RNA in the extracted tissue. Primers that amplify the NLuc ORF are used (F: AGATTTCGTTGGGGACTGGC (SEQ ID NO: 7), R: CACCGCTCAGGACAATCCTT (SEQ ID NO: 8)). To detect circular RNA only, primers that amplify the 5′-3′ junction allow for detection of circular but not linear RNA constructs (F: CTGGAGACGTGGAGGAGAAC (SEQ ID NO: 9), R: CCAAAAGACGGCAATATGGT (SEQ ID NO: 10)).

It is expected that linear and circular RNA will be detected in nasal tissue samples at 6, 24 and 48 hours after topical administration of linear and circular RNA and will show greater expression than the vehicle only control.

This Example describes that RNA is successfully delivered via topical administration to the nasal mucosal epithelium and persists in tissue over prolonged periods of time.

### Example 14: Topical Administration of mRNA Results in RNA Delivery to Tissue when Tissue is Wiped with an Ethanol Wipe Prior to Application

This Example demonstrates the ability to delivery mRNA to cells and tissues via topical administration in vivo.

In this example, mRNA was designed with an ORF encoding a Nano Luciferase (NLuc). In this example, modified linear mRNA was made in-house by in vitro transcription. In this example, RNA was fully substituted with Pseudo-Uridine and 5-Methyl-C, capped with CleanCap™ AG, included 5′ and 3′ human alpha-globin UTRs, and is polyadenylated.

RNA was then diluted in PBS only, or with PBS and 10% (v/v) ethanol, such that total sample volume for each sample was 35 uL, and total RNA for each sample was 20 picomoles. As negative controls, vehicle only controls were prepared as described above but without RNA. All reagents are brought to room temperature prior to mixing and mixtures are prepared immediately prior to use.

At time=0, the ear of the mouse was wiped with a cotton swab dipped in 70% ethanol, dried and a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

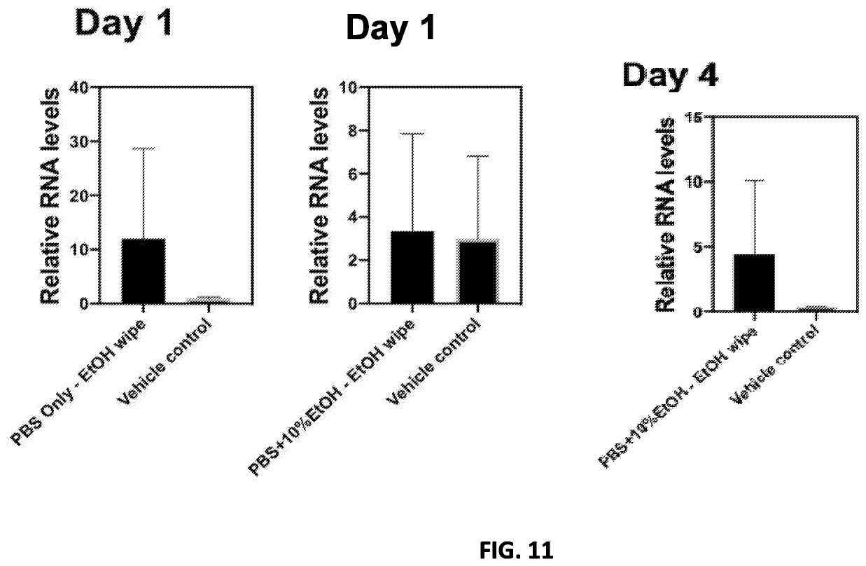

mRNA was detected in tissue samples at 1 day for mRNA in PBS only, and at 1 and 4 days for mRNA in PBS+10% EtOH after topical administration and showed greater signal than the negative controls (FIG. 11).

This Example demonstrates that mRNA is successfully delivered via topical administration when the skin is wiped with an ethanol wipe prior to administration and persists in tissue over prolonged periods of time.

### Example 15: Topical Administration of mRNA Results in RNA Delivery to Tissue when Tissue is Wiped with an Isopropyl Alcohol Wipe Prior to Application

This Example demonstrates the ability to delivery mRNA to cells and tissues via topical administration in vivo.

In this example, mRNA was designed with an ORF encoding a Nano Luciferase (NLuc). In this example, modified linear mRNA was made in-house by in vitro transcription. In this example, RNA was fully substituted with Pseudo-Uridine and 5-Methyl-C, capped with CleanCap™ AG, included 5′ and 3′ human alpha-globin UTRs, and is polyadenylated.

RNA was then diluted in PBS only such that total sample volume for each sample was 35 uL, and total RNA for each sample was 20 picomoles. As negative controls, vehicle only controls were prepared as described above but without RNA. All reagents are brought to room temperature prior to mixing and mixtures are prepared immediately prior to use.

At time=0, the ear of the mouse was wiped with a commercial isopropyl alcohol wipe (CVS, 297584), dried and a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

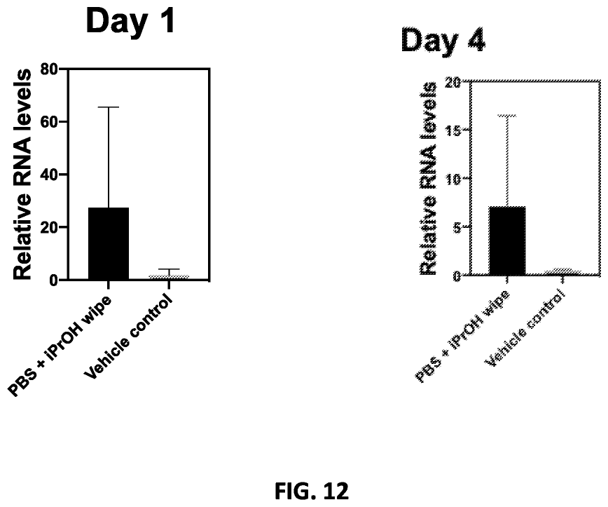

mRNA was detected in tissue samples at 1 and 4 days after topical administration and showed greater signal than the negative controls (FIG. 12).

This Example demonstrates that mRNA is successfully delivered via topical administration when the skin is wiped with an isopropyl alcohol wipe prior to administration and persists in tissue over prolonged periods of time.

### Example 16: Topical Administration of Circular RNA Results in RNA Delivery to Tissue when Tissue is Wiped with an Ethanol Wipe Prior to Application

This Example demonstrates the ability to delivery unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in either PBS only, or PBS with 10% (v/v) ethanol, such that total sample volume for each sample was 35 uL, and total RNA for each sample was 20 pmoles. As negative controls, vehicle only controls were prepared as described above but without RNA. All reagents are brought to room temperature prior to mixing and mixtures are prepared immediately prior to use.

At time=0, the ear of the mouse was wiped with an ethanol wipe, dried and a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

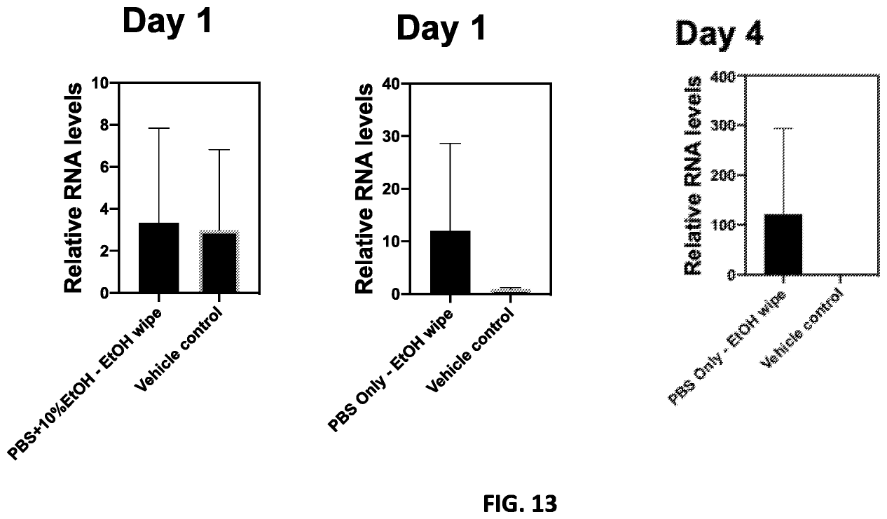

Circular RNA was detected in tissue samples at 1 and 4 days for circular RNA in PBS only, and at 1 days for circular RNA in PBS+10% EtOH after topical administration following wiping the skin with an ethanol wipe and showed greater signal than the relevant vehicle only control (FIG. 13).

This Example demonstrates that circular RNA is successfully delivered via topical administration when the skin is wiped with an ethanol wipe prior to administration and persists in tissue over prolonged periods of time.

### Example 17: Topical Administration of Circular RNA Results in RNA Delivery to Tissue

This Example demonstrates the ability to delivery unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS with 10% (v/v) ethanol, such that total sample volume for each sample was 35 uL, and total RNA for each sample was 20 pmoles. As negative controls, vehicle only controls were prepared as described above but without RNA. All reagents were brought to room temperature prior to mixing and mixtures were prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 day and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

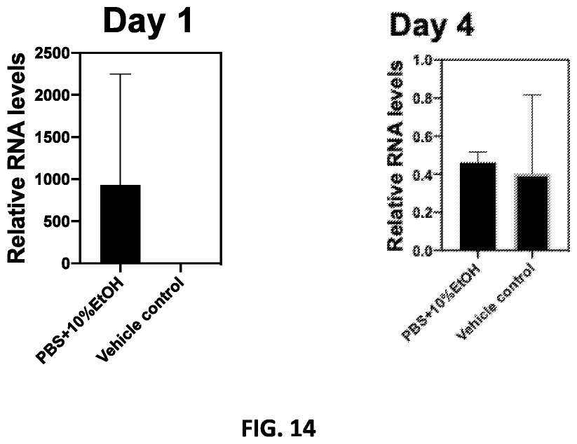

Circular RNA was detected in tissue samples at 1 day and 4 days after topical administration and showed greater signal than the relevant negative control (FIG. 14).

This Example demonstrates that circular RNA is successfully delivered to tissues via topical administration to the skin and persists in tissue over prolonged periods of time.

### Example 18: Topical Administration of Circular RNA Results in RNA Delivery to Tissue

This Example demonstrates the ability to delivery unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS with 10% (v/v) isopropyl alcohol, such that total sample volume for each sample was 35 uL, and total RNA for each sample was 20 pmoles. As negative controls, vehicle only controls were prepared as described above but without RNA. All reagents were brought to room temperature prior to mixing and mixtures were prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 day and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

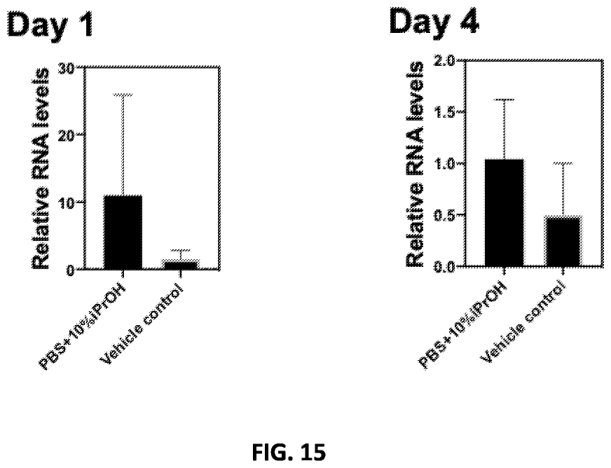

Circular RNA was detected in tissue samples at 1 day and 4 days after topical administration and showed greater signal than the relevant negative control (FIG. 15).

This Example demonstrates that endless RNA is successfully delivered to tissues via topical administration to the skin and persists in tissue over prolonged periods of time.

### Example 19: Topical Administration of Circular RNA Results in RNA Delivery to Tissue when Tissue is Wiped with an Isopropyl Alcohol Wipe Prior to Application

This Example demonstrates the ability to delivery unmodified circular RNA to cells and tissues via topical administration in vivo.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS only such that total sample volume for each sample was 35 uL, and total RNA for each sample was 20 pmoles. As negative controls, vehicle only controls were prepared as described above but without RNA. All reagents are brought to room temperature prior to mixing and mixtures are prepared immediately prior to use.

At time=0, the ear of the mouse was wiped with an isopropyl alcohol wipe (CVS, 297584), dried and a 35 μL dose of each sample was applied topically to the ear of a BALB/c mouse dropwise using a pipet tip. As a negative control, an isopropyl alcohol wipe alone was used. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 day and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

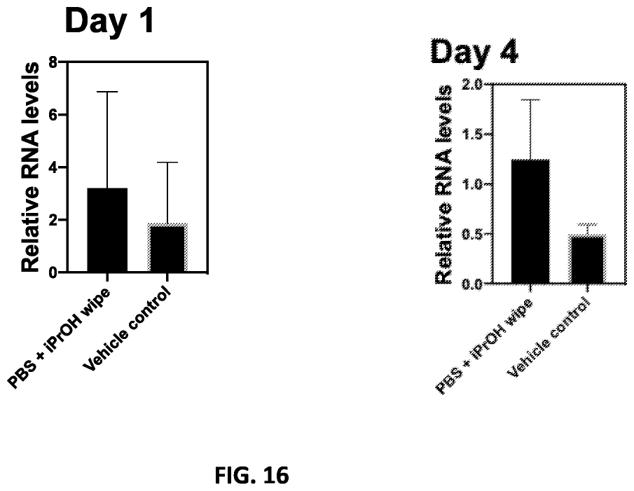

Circular RNA was detected in tissue samples at 1 day and 4 days after topical administration and showed greater signal than the vehicle only control (FIG. 16).

This Example demonstrates that endless RNA is successfully delivered via topical administration to the tissue after wiping the skin with an isopropyl alcohol wipe and persists in tissue over prolonged periods of time.

### Example 20: Topical Administration of Linear mRNA Results in RNA Delivery to Tissue

This Example demonstrates the ability to delivery mRNA to cells and tissues via topical administration in vivo.

In this example, mRNA was designed with an ORF encoding a Nano Luciferase (NLuc). In this example, modified linear mRNA was made in-house by in vitro transcription. In this example, RNA was fully substituted with Pseudo-Uridine and 5-Methyl-C, capped with CleanCap™ AG, included 5′ and 3′ human alpha-globin UTRs, and is polyadenylated.

RNA was diluted in (1) PBS only, (2) PBS with 10% (v/v) ethanol, (3) PBS with 10% (v/v) isopropyl alcohol, such that total sample volume for each sample was 35 uL, and total RNA for each sample was 20 pmoles. As negative controls, vehicle only controls were prepared as described above but without RNA. All reagents are brought to room temperature prior to mixing and mixtures are prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA persistence in tissue, tissue samples were analyzed for RNA at varying timepoints after delivery using RT-qPCR. At 1 day and 4 days post-administration, a 2 mm ear punch was taken from each animal and stored in RNAlater solution (ThermoFisher Scientific, cat #AM7020). Total RNA was isolated from ear punches by snap-cooling and homogenizing the tissue in liquid nitrogen with a glass mortar and pestle followed by trizol extraction (ThermoFisher Scientific cat #15596026). The aqueous-phase was precipitated with isopropanol and the pellet was washed with 70% ETOH as per manufacturer's instructions. cDNA was synthesized from the total RNA using Superscript IV (Thermo Scientific, cat #11766500). RT-PCR was performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples were assayed in triplicate on the Bio-rad CFX384 Thermal Cycler. RNA levels were then relativized to housekeeping gene (28s).

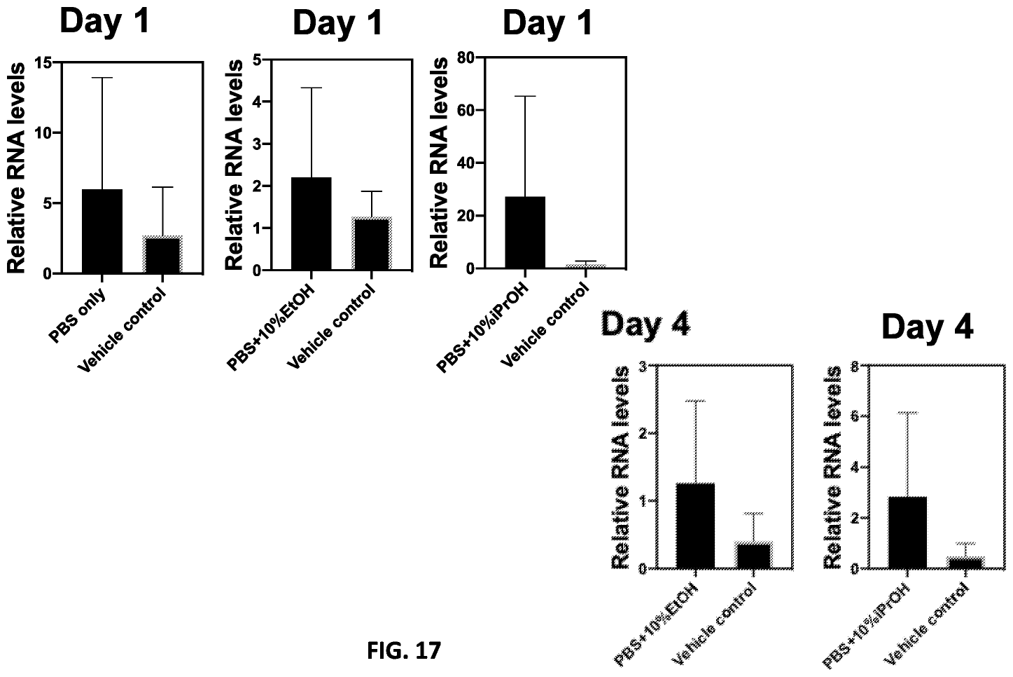

mRNA was detected in tissue samples at 1 day for mRNA in PBS only, and at 1 day and 4 days for both mRNA in PBS+10% EtOH and mRNA in PBS+10% iPrOH after topical administration and showed greater signal than the relevant negative control (FIG. 17).

This Example demonstrates that mRNA is successfully delivered to tissues via topical administration to the skin and persists in tissue over prolonged periods of time.

### Example 21: Topical Administration of Circular RNA Results in Protein Expression in Tissue when Tissue is Wiped with an Ethanol Wipe Prior to Application

This Example demonstrates the ability to deliver unmodified circular RNA to cells and tissues via topical administration in vivo and achieve subsequent protein expression.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS only such that total sample volume for each sample was 35 uL, and total RNA for each sample was 20 pmoles. Vehicle only control samples were prepared similarly but without RNA. All reagents were brought to room temperature prior to mixing and mixtures were prepared immediately prior to use.

At time=0, the ear of the mouse was wiped with an ethanol wipe, dried and a 35 dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 uL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 uL of furimazine substrate and 49 uL of PBS were added to each well and mixed. Plates were incubated for 10 min after substrate addition and mixing and then read in a luminometer instrument (Promega).

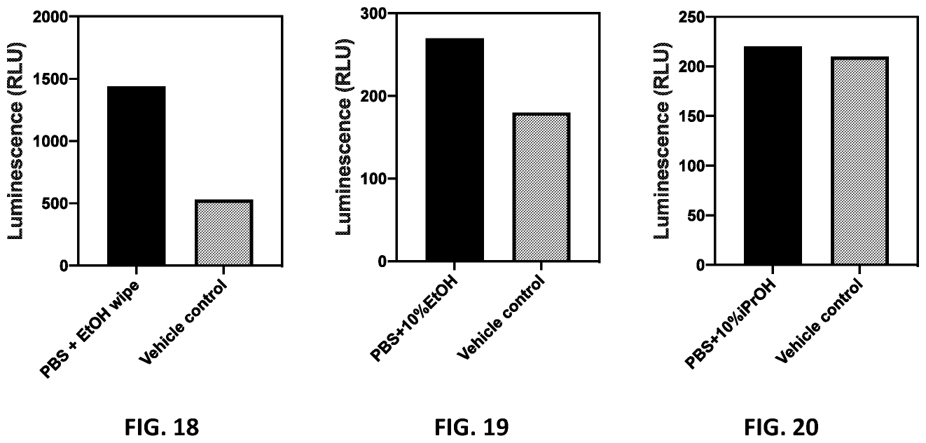

Nano Luciferase activity was detected in tissue samples at 4 days after topical administration for circular RNA in PBS only and was observed to be higher than the relevant vehicle only control (FIG. 18).

This Example demonstrated that circular RNA was successfully delivered via topical administration and was able to express functional protein, detectable in tissue for prolonged periods of time.

### Example 22: Topical Administration of Circular RNA Results in Protein Expression in Tissue

This Example demonstrates the ability to deliver unmodified circular RNA to cells and tissues via topical administration in vivo and achieve subsequent protein expression.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS with 10% (v/v) ethanol, such that total sample volume for each sample was 35 uL, and total RNA for each sample was 20 pmoles. As negative controls, vehicle only controls were prepared as described above but without RNA. All reagents were brought to room temperature prior to mixing and mixtures were prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 uL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 uL of furimazine substrate and 49 uL of PBS were added to each well and mixed. Plates were incubated for 10 min after substrate addition and mixing and then read in a luminometer instrument (Promega).

Nano Luciferase activity was detected in tissue samples at 4 days after topical administration for circular RNA in PBS with 10% ethanol (v/v) and was observed to be higher than the relevant vehicle only control (FIG. 19).

This Example demonstrated that circular RNA was successfully delivered via topical administration and was able to express functional protein, detectable in tissue for prolonged periods of time.

### Example 23: Topical Administration of Circular RNA Results in Protein Expression in Tissue

This Example demonstrates the ability to delivery unmodified circular RNA to cells and tissues via topical administration in vivo and achieve subsequent protein expression.

In this example, circular RNA was designed with an IRES and ORF encoding Nanoluciferase (NLuc).

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA will be circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA was then diluted in PBS with 10% (v/v) isopropyl alcohol, such that total sample volume for each sample was 35 uL, and total RNA for each sample was 20 pmoles. As negative controls, vehicle only controls were prepared as described above but without RNA. All reagents were brought to room temperature prior to mixing and mixtures were prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 uL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 uL of furimazine substrate and 49 uL of PBS were added to each well and mixed. Plates were incubated for 10 min after substrate addition and mixing and then read in a luminometer instrument (Promega).

Nano Luciferase activity was detected in tissue samples at 4 days after topical administration for circular RNA in PBS with 10% isopropyl alcohol and was observed to be higher than the relevant vehicle only control (FIG. 20).

This Example demonstrated that circular RNA was successfully delivered via topical administration and was able to express functional protein, detectable in tissue for prolonged periods of time.

### Example 24: Topical Administration of Linear mRNA Results in RNA Delivery to Tissue and Subsequent Protein Expression

This Example demonstrates the ability to deliver mRNA to cells and tissues via topical administration in vivo and achieve subsequent protein expression.

In this example, mRNA was designed with an ORF encoding a Nano Luciferase (NLuc). In this example, modified linear mRNA was made in-house by in vitro transcription. In this example, RNA was fully substituted with Pseudo-Uridine and 5-Methyl-C, capped with CleanCap™ AG, included 5′ and 3′ human alpha-globin UTRs, and is polyadenylated.

RNA was diluted in PBS with 10% (v/v) ethanol, such that total sample volume for each sample was 35 uL, and total RNA for each sample was 20 pmoles. As negative controls, vehicle only controls were prepared as described above but without RNA. All reagents are brought to room temperature prior to mixing and mixtures are prepared immediately prior to use.

At time=0, the ear of the mouse was wiped with an ethanol wipe, dried and a 35 dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 uL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 uL of furimazine substrate and 49 uL of PBS were added to each well and mixed. Plates were incubated for 10 min after substrate addition and mixing and then read in a luminometer instrument (Promega).

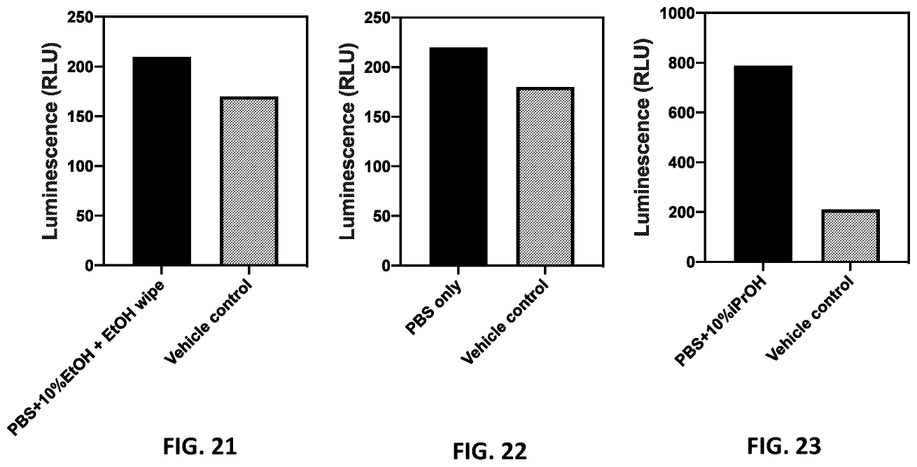

Nano Luciferase activity was detected in tissue samples at 4 days after topical administration for circular RNA in PBS with 10% ethanol and was observed in each case to be higher than the relevant vehicle only control (FIG. 21).

This Example demonstrates that mRNA is successfully delivered to tissues via topical administration to the skin when the skin is wiped with an ethanol wipe before administration and persists in tissue over prolonged periods of time and is able to express functional protein.

### Example 25: Topical Administration of Linear mRNA Results in RNA Delivery to Tissue and Subsequent Protein Expression

This Example demonstrates the ability to deliver mRNA to cells and tissues via topical administration in vivo and achieve subsequent protein expression.

In this example, mRNA was designed with an ORF encoding a Nano Luciferase (NLuc). In this example, modified linear mRNA was made in-house by in vitro transcription. In this example, RNA was fully substituted with Pseudo-Uridine and 5-Methyl-C, capped with CleanCap™ AG, included 5′ and 3′ human alpha-globin UTRs, and is polyadenylated.

RNA was diluted in (1) PBS only, or (2) PBS with 10% (v/v) isopropyl alcohol, such that total sample volume for each sample was 35 uL, and total RNA for each sample was 20 pmoles. As negative controls, vehicle only controls were prepared as described above but without RNA. All reagents are brought to room temperature prior to mixing and mixtures are prepared immediately prior to use.

At time=0, a 35 μL dose of each sample was applied topically to the ear of a BALB/c mouse dropwise using a pipet tip. Samples were dried by exposing the ears briefly to a heat lamp and fan in a sterilized hood. Mice were place back in cages under normal conditions.

To determine RNA expression in tissue, tissue samples were analyzed for NLuc activity at varying timepoints after topical delivery. Ear punches were taken from the mouse at 4 days after delivery. Each tissue sample was crushed into fragments and was placed in 50 μL of ice cold NLuc Lysis Assay Buffer with 1× Protease Inhibitor Cocktail and placed on ice. Samples were then incubated on an orbital shaker for 5 minutes at 700 rpm, and then centrifuged at room temperature to remove tissue debris. The 50 uL supernatant was then transferred to a fresh tube without disturbing the tissue pellet. 50 μL of each sample was transferred to a 96 well plate and Nano-Glo Luciferase Assay System (Promega, #N1110) assay was performed according to manufacturer's instruction. In brief, 1 uL of furimazine substrate and 49 uL of PBS were added to each well and mixed. Plates were incubated for 10 min after substrate addition and mixing and then read in a luminometer instrument (Promega).

Nano Luciferase activity was detected in tissue samples at 4 days after topical administration for linear mRNA in PBS only, and linear mRNA in PBS with 10% isopropyl alcohol and was observed in each case to be higher than the relevant vehicle only control (FIG. 22 and FIG. 23).

This Example demonstrates that mRNA is successfully delivered to tissues via topical administration to the skin and persists in tissue over prolonged periods of time and is able to express functional protein.

### Example 26: Topical Administration of Circular RNA Results in RNA Delivery to Tissue when Tissue is Wiped with a Povidone Iodine Prior to Application

This Example describes the ability to deliver unmodified circular RNA to cells and tissues via topical administration in vivo.

In this example, circular RNA is designed with an IRES and ORF encoding Nanoluciferase (NLuc).

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), is treated with RNA 5′phosphohydrolase (RppH) (New England Biolabs, M0356) following the manufacturer's instructions, and is purified again with an RNA purification column. RppH treated linear RNA is circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA is Urea-PAGE purified, eluted in a buffer (0.5 M Sodium Acetate, 0.1% SDS, 1 mM EDTA), is ethanol precipitated and is resuspended in RNA storage solution (ThermoFisher Scientific, cat #AM7000).

RNA is then diluted in PBS only such that total sample volume for each sample is 35 uL, and total RNA for each sample is 20 pmoles. As negative controls, vehicle only controls are prepared as described above but without RNA. All reagents are brought to room temperature prior to mixing and mixtures are prepared immediately prior to use.

At time=0, the ear of the mouse is wiped with commercial povidone iodine (10%), which is a sterilizing agent. Excess povidone iodine is removed with a sterile cotton swab and a 35 μL dose of each sample is applied topically to the ear of a BALB/c mouse dropwise using a pipet tip. Samples are dried by exposing the ears briefly to a heat lamp and fan in a sterilized hood. Mice are placed back in cages under normal conditions.

To determine RNA persistence in tissue, tissue samples are analyzed for RNA at varying timepoints after delivery using RT-qPCR. At 1 day and 4 days post-administration, a 2 mm ear punch is taken from each animal and stored in RNAlater solution (ThermoFisher Scientific, cat #AM7020). Total RNA is isolated from ear punches by snap-cooling and homogenizing the tissue in liquid nitrogen with a glass mortar and pestle followed by trizol extraction (ThermoFisher Scientific cat #15596026). The aqueous-phase is precipitated with isopropanol and the pellet is washed with 70% ETOH as per manufacturer's instructions. cDNA is synthesized from the total RNA using Superscript IV (Thermo Scientific, cat #11766500). RT-PCR is performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples are assayed in triplicate on the Bio-rad CFX384 Thermal Cycler. RNA levels are then be relativized to housekeeping gene (28s).

It is expected that circular RNA is detected in tissue samples at 1 day and 4 days after topical administration and greater signal than the vehicle only control is observed.

This Example describes that circular RNA is successfully delivered via topical administration to the tissue after wiping the skin with povidone iodine (10%) and persists in tissue over prolonged periods of time.

### Example 27: Topical Administration of Circular RNA Results in RNA Delivery to Tissue when Tissue is Sprayed with a Hydrogen Peroxide Spray Prior to Application

This Example describes the ability to deliver unmodified circular RNA to cells and tissues via topical administration in vivo.

In this example, circular RNA is designed with an IRES and ORF encoding Nanoluciferase (NLuc).

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA will be circularized using a splint DNA (5′-TTTTTCGGCTATTCCCAATAGCCGTTTTG-3′ (SEQ ID NO: 11)) and T4 RNA ligase 2 (New England Biolabs, M0239). Circular RNA is Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

RNA is then diluted in PBS only such that total sample volume for each sample is 35 uL, and total RNA for each sample is 20 pmoles. As negative controls, vehicle only controls are prepared as described above but without RNA. All reagents are brought to room temperature prior to mixing and mixtures are prepared immediately prior to use.

At time=0, the ear of the mouse is sprayed with commercial hydrogen peroxide (3%), which is a sterilizing agent, dried with a sterile cotton swab and a 35 μL dose of each sample is applied topically to the ear of a BALB/c mouse dropwise using a pipet tip. Samples are dried by exposing the ears briefly to a heat lamp and fan in a sterilized hood. Mice are placed back in cages under normal conditions.

To determine RNA persistence in tissue, tissue samples are analyzed for RNA at varying timepoints after delivery using RT-qPCR. At 1 day and 4 days post-administration, a 2 mm ear punch is taken from each animal and stored in RNAlater solution (ThermoFisher Scientific, cat #AM7020). Total RNA is isolated from ear punches by snap-cooling and homogenizing the tissue in liquid nitrogen with a glass mortar and pestle followed by trizol extraction (ThermoFisher Scientific cat #15596026). The aqueous-phase is precipitated with isopropanol and the pellet is washed with 70% ETOH as per manufacturer's instructions. cDNA is synthesized from the total RNA using Superscript IV (Thermo Scientific, cat #11766500). RT-PCR is performed on cDNA templates using iTaq™ Universal SYBR® Green Supermix (Bio-rad, catalog #1725124) and primers specific to the NLuc ORF (F: CCGTATGAAGGTCTGAGCGG (SEQ ID NO: 12), R: CAGTGTGCCATAGTGCAGGA (SEQ ID NO: 13)). All samples are assayed in triplicate on the Bio-rad CFX384 Thermal Cycler. RNA levels are then relativized to housekeeping gene (28s).

It is expected that circular RNA is detected in tissue samples at 1 day and 4 days after topical administration and greater signal than the vehicle only control is observed.

This Example describes that circular RNA is successfully delivered via topical administration to the tissue after spraying the skin with hydrogen peroxide (3%) and persists in tissue over prolonged periods of time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

SEQUENCES

Nano Luciferase DNA template
SEQ ID NO: 1
GTCTTCACACTCGAAGATTTCGTTGGGGACTGGCGACAGACAGCCGGCT
ACAACCTGGACCAAGTCCTTGAACAGGGAGGTGTGTCCAGTTTGTTTCA
GAATCTCGGGGTGTCCGTAACTCCGATCCAAAGGATTGTCCTGAGCGGT
GAAAATGGGCTGAAGATCGACATCCATGTCATCATCCCGTATGAAGGTC
TGAGCGGCGACCAAATGGGCCAGATCGAAAAAATTTTTAAGGTGGTGTA
CCCTGTGGATGATCATCACTTTAAGGTGATCCTGCACTATGGCACACTG
GTAATCGACGGGGTTACGCCGAACATGATCGACTATTTCGGACGGCCGT
ATGAAGGCATCGCCGTGTTCGACGGCAAAAAGATCACTGTAACAGGGAC
CCTGTGGAACGGCAACAAAATTATCGACGAGCGCCTGATCAACCCCGAC
GGCTCCCTGCTGTTCCGAGTAACCATCAACGGAGTGACCGGCTGGCGGC
TGTGCGAACGCATTCTGGCG
EMCV IRES
SEQ ID NO: 2
ACGTTACTGGCCGAAGCCGCTTGGAATAAGGCCGGTGTGCGTTTGTCTA
TATGTTATTTTCCACCATATTGCCGTCTTTTGGCAATGTGAGGGCCCGG
AAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGG
CAGCGGAACCCCCCACCTGGCGACAGGTGCCTCTGCGGCCAAAAGCCAC
GTGTATAAGATACACCTGCAAAGGCGGCACAACCCCAGTGCCACGTTGT
GAGTTGGATAGTTGTGGAAAGAGTCAAATGGCTCTCCTCAAGCGTATTC
AACAAGGGGCTGAAGGATGCCCAGAAGGTACCCCATTGTATGGGATCTG
ATCTGGGGCCTCGGTGCACATGCTTTACATGTGTTTAGTCGAGGTTAAA
AAACGTCTAGGCCCCCCGAACCACGGGGACGTGGTTTTCCTTTGAAAAA
CACGATGATAATA
Gaussia Luciferase DNA template
SEQ ID NO: 3
ATGGGAGTCAAAGTTCTGTTTGCCCTGATCTGCATCGCTGTGGCCGAGG
CCAAGCCCACCGAGAACAACGAAGACTTCAACATCGTGGCCGTGGCCAG
CAACTTCGCGACCACGGATCTCGATGCTGACCGCGGGAAGTTGCCCGGC
AAGAAGCTGCCGCTGGAGGTGCTCAAAGAGATGGAAGCCAATGCCCGGA
AAGCTGGCTGCACCAGGGGCTGTCTGATCTGCCTGTCCCACATCAAGTG
CACGCCCAAGATGAAGAAGTTCATCCCAGGACGCTGCCACACCTACGAA
GGCGACAAAGAGTCCGCACAGGGCGGCATAGGCGAGGCGATCGTCGACA
TTCCTGAGATTCCTGGGTTCAAGGACTTGGAGCCCATGGAGCAGTTCAT
CGCACAGGTCGATCTGTGTGTGGACTGCACAACTGGCTGCCTCAAAGGG
CTTGCCAACGTGCAGTGTTCTGACCTGCTCAAGAAGTGGCTGCCGCAAC
GCTGTGCGACCTTTGCCAGCAAGATCCAGGGCCAGGTGGACAAGATCAA
GGGGGCCGGTGGTGACTAA
EPO DNA template
SEQ ID NO: 4
ATGGGAGTGCACGAGTGTCCCGCGTGGTTGTGGTTGCTGCTGTCGCTCT
TGAGCCTCCCACTGGGACTGCCTGTGCTGGGGGCACCACCCAGATTGAT
CTGCGACTCACGGGTACTTGAGAGGTACCTTCTTGAAGCCAAAGAAGCC
GAAAACATCACAACCGGATGCGCCGAGCACTGCTCCCTCAATGAGAACA
TTACTGTACCGGATACAAAGGTCAATTTCTATGCATGGAAGAGAATGGA
AGTAGGACAGCAGGCCGTCGAAGTGTGGCAGGGGCTCGCGCTTTTGTCG
GAGGCGGTGTTGCGGGGTCAGGCCCTCCTCGTCAACTCATCACAGCCGT
GGGAGCCCCTCCAACTTCATGTCGATAAAGCGGTGTCGGGGCTCCGCAG
CTTGACGACGTTGCTTCGGGCTCTGGGCGCACAAAAGGAGGCTATTTCG
CCGCCTGACGCGGCCTCCGCGGCACCCCTCCGAACGATCACCGCGGACA
CGTTTAGGAAGCTTTTTAGAGTGTACAGCAATTTCCTCCGCGGAAAGCT
GAAATTGTATACTGGTGAAGCGTGTAGGACAGGGGATCGC
CVB3 IRES DNA template
SEQ ID NO: 5
TTAAAACAGCCTGTGGGTTGATCCCACCCACAGGCCCATTGGGCGCTAG
CACTCTGGTATCACGGTACCTTTGTGCGCCTGTTTTATACCCCCTCCCC
CAACTGTAACTTAGAAGTAACACACACCGATCAACAGTCAGCGTGGCAC
ACCAGCCACGTTTTGATCAAGCACTTCTGTTACCCCGGACTGAGTATCA
ATAGACTGCTCACGCGGTTGAAGGAGAAAGCGTTCGTTATCCGGCCAAC
TACTTCGAAAAACCTAGTAACACCGTGGAAGTTGCAGAGTGTTTCGCTC
AGCACTACCCCAGTGTAGATCAGGTCGATGAGTCACCGCATTCCCCACG
GGCGACCGTGGCGGTGGCTGCGTTGGCGGCCTGCCCATGGGGAAACCCA
TGGGACGCTCTAATACAGACATGGTGCGAAGAGTCTATTGAGCTAGTTG
GTAGTCCTCCGGCCCCTGAATGCGGCTAATCCTAACTGCGGAGCACACA
CCCTCAAGCCAGAGGGCAGTGTGTCGTAACGGGCAACTCTGCAGCGGAA
CCGACTACTTTGGGTGTCCGTGTTTCATTTTATTCCTATACTGGCTGCT
TATGGTGACAATTGAGAGATCGTTACCATATAGCTATTGGATTGGCCAT
CCGGTGACTAATAGAGCTATTATATATCCCTTTGTTGGGTTTATACCAC
TTAGCTTGAAAGAGGTTAAAACATTACAATTCATTGTTAAGTTGAATAC
AGCAAA
Green Fluorescent Protein DNA template
SEQ ID NO: 6
AGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGA
GGGCGATGCCACCTACGGCAAGCTGACCCTGAAGTTCATCTGCACCACC
GGCAAGCTGCCCGTGCCCTGGCCCACCCTCGTGACCACCCTGACCTACG
GCGTGCAGTGCTTCAGCCGCTACCCCGACCACATGAAGCAGCACGACTT
CTTCAAGTCCGCCATGCCCGAAGGCTACGTCCAGGAGCGCACCATCTTC
TTCAAGGACGACGGCAACTACAAGACCCGCGCCGAGGTGAAGTTCGAGG
GCGACACCCTGGTGAACCGCATCGAGCTGAAGGGCATCGACTTCAAGGA
GGACGGCAACATCCTGGGGCACAAGCTGGAGTACAACTACAACAGCCAC
AACGTCTATATCATGGCCGACAAGCAGAAGAACGGCATCAAGGTGAACT
TCAAGATCCGCCACAACATCGAGGACGGCAGCGTGCAGCTCGCCGACCA
CTACCAGCAGAACACCCCCATCGGCGACGGCCCCGTGCTGCTGCCCGAC
AACCACTACCTGAGCACCCAGTCCGCCCTGAGCAAAGACCCCAACGAGA
AGCGCGATCACATGGTCCTGCTGGAGTTCGTGACCGCCGCCGGGATCAC
TCTCGGCATGGACGAGCTGTACAAG

What is claimed is:

1. A pharmaceutical composition comprising a mixture of a polyribonucleotide and ethanol, wherein the ethanol constitutes:
(i) at least about 0.3% v/v to about 75% v/v of the mixture; or
(ii) at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture; or
(iii) at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

2. The pharmaceutical composition of claim 1, wherein the polyribonucleotide encodes a protein.

3. The pharmaceutical composition of claim 2, wherein the protein is a therapeutic protein.

4. The pharmaceutical composition of claim 2, wherein the protein is a wound healing protein, e.g., a growth factor.

5. The pharmaceutical composition of claim 4, wherein the growth factor is EGF, PDGF, TGFβ, or VEGF.

6. The pharmaceutical composition of any one of claims 1-5, wherein the pharmaceutical composition is a liquid, gel, lotion, paste, cream, foam, or stick.

7. The pharmaceutical composition of any one of claims 1-6, wherein the polyribonucleotide is a linear polyribonucleotide or an mRNA, and optionally, wherein the polyribonucleotide lacks a cap or poly-A tail.

8. The pharmaceutical composition of any one of claims 1-6, wherein the polyribonucleotide is a circular polyribonucleotide, and optionally comprises a modified ribonucleotide.

9. The pharmaceutical composition of any one of claims 1-8, wherein the pharmaceutical composition:
(i) has a pH of about 7; and/or
(ii) has a viscosity that is about the same as water; and/or
(iii) is substantially free of hydrophobic or lipophilic groups; and/or
(iv) is substantially free of hydrocarbons; and/or
(v) is substantially free of cationic liposomes; and/or
(vi) is substantially free of fatty acids, lipids, liposomes, cholesterol, or any combination thereof.

10. A method of delivering a polyriboribonucleotide to a subject comprising topically applying a composition comprising a mixture of a polyribonucleotide and ethanol to a surface area of the subject, wherein the ethanol constitutes at least about 0.3% v/v to about 75% v/v of the mixture.

11. A method of delivering a polyribonucleotide to a subject comprising a) applying a sterilizing agent to a surface area of the subject;
b) applying a composition free of any carrier comprising the polyribonucleotide and diluent to the surface area.

12. The method of claim 11, wherein the sterilizing agent is an alcohol, UV light, laser light, or heat.

13. A method of delivering a polyribonucleotide to a subject comprising
a) applying an alcohol to a surface area of the subject;
b) applying a composition free of any carrier comprising the polyribonucleotide and diluent to the surface.

14. The method of claim 13, wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

15. The method of claim 13, wherein the alcohol comprises ethanol.

16. A method of delivering a polyribonucleotide to an epithelial cell comprising applying a composition free of any carrier comprising a diluent and a polyribonucleotide that is not modified to the epithelial cell.

17. A method of delivering a polyribonucleotide to a subject comprising topically applying a composition comprising a mixture of a polyribonucleotide and an alcohol to a surface area of the subject, wherein the alcohol constitutes at least about 0.3% v/v to about 75% v/v of the mixture.

18. A method of delivering a polyribonucleotide to a subject comprising topically applying a composition comprising a mixture of a polyribonucleotide and a cell-penetrating agent to a surface area of the subject, wherein the cell-penetrating agent constitutes at least about 0.3% v/v to about 75% v/v of the mixture.

19. The method of any one of claim 18, wherein the composition delivers the polyribonucleotide to a dermal or epidermal tissue of the subject, and optionally, without iontophoresis.

20. A kit comprising an application tool and the pharmaceutical composition of any one of claims 1-9, wherein the application tool is configured to apply the pharmaceutical composition to a surface area of a subject.

21. A kit comprising a first application tool, a second application tool, a sterilizing agent, and a composition free of any carrier comprising the polyribonucleotide and diluent, wherein the first application tool is configured to apply a sterilizing agent to a surface area of a subject and the second application tool is configured to apply the composition to the surface area of the subject.

22. The kit of claim 21, wherein the sterilizing agent is an alcohol, UV light, laser light, or heat.

23. The kit of claim 22, wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

24. The kit of any one of claims 20-23, wherein the first application tool is a wipe, and optionally, the wipe comprises the sterilizing agent.

25. The kit of claim 20 or 21, wherein first application tool is (i) a device that applies UV light or laser light; or (ii) a device that applies heat.

26. A kit comprising an application tool and a mixture comprising a polyribonucleotide and a cell-penetrating agent, wherein the application tool is configured to apply the mixture to a surface area of a subject.

27. The kit of any one of claims 20-26, wherein the application tool or second application tool comprises a pipette.

28. The kit of any one of claims 20-26, wherein the application tool or second application tool comprises a substrate, and wherein the substrate is embedded with the mixture, and optionally, wherein the substrate is made of natural or artificial fibers, and optionally, the kit comprises a suppository.

29. The kit of any one of claims 20-28, wherein the application tool or second application tool comprises: (i) a patch; (ii) a sprayer; (iii) a nebulizer; or (iv) a capsule configured to release the mixture inside gastrointestinal tract of the subject, and optionally, the application tool or second application tool is configured to release the mixture in a controlled manner.

30. The kit of any one of claims 20-29, wherein the surface area is selected from the group consisting of: skin, surface areas of oral cavity, nasal cavity, gastrointestinal tract, and respiratory tract, and any combination thereof.

31. A pharmaceutical composition comprising a mixture of a polyribonucleotide and an alcohol, wherein the alcohol constitutes:
(i) at least about 0.3% v/v to about 75% v/v of the mixture; or
(ii) at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, or at least about 0.3% v/v to about 1% v/v, or at least about 0.3% v/v to about 0.5% v/v of the mixture; or
(iii) at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

32. The pharmaceutical composition of claim 31, wherein the alcohol is selected from the group consisting of: methanol, ethanol, isopropanol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, and hydroxyethylcellulose.

33. A pharmaceutical composition comprising a mixture of a polyribonucleotide and a cell-penetrating agent, wherein the cell-penetrating agent constitutes:
(i) at least about 0.3% v/v to about 75% v/v of the mixture; or
(ii) at least about 0.3% v/v to about 70% v/v, at least about 0.3% v/v to about 60% v/v, at least about 0.3% v/v to about 50% v/v, at least about 0.3% v/v to about 40% v/v, at least about 30% v/v to about 20% v/v, at least about 0.3% v/v to about 15% v/v, at least about 0.3% v/v to about 10% v/v, at least about 0.3% v/v to about 5% v/v, at least about 0.3% v/v to about 1% v/v, or at least about 0.3% v/v to about 0.5% v/v of the mixture; or
(iii) at least about 0.5% v/v to about 75% v/v, at least about 1% v/v to about 75% v/v, at least about 5% v/v to about 75% v/v, at least about 10% v/v to about 75% v/v, at least about 15% v/v to about 75% v/v, at least about 20% v/v to about 75% v/v, at least about 30% v/v to about 75% v/v, at least about 40% v/v to about 75% v/v, at least about 50% v/v to about 75% v/v, at least about 60% v/v to about 75% v/v, or at least about 70% v/v to about 75% v/v of the mixture.

34. The pharmaceutical composition of claim 33, wherein the cell penetrating agent is: (i) soluble in polar solvents; or (ii) insoluble in polar solvents.


SEQUENCE LISTING (13 sequences)

SEQ ID NO: 1 - DNA, 510 bp, Artificial Sequence
  source: /note=“Description of Artificial Sequence Synthetic polynucleotide”
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 2 - DNA, 552 bp, Encephalomyocarditis virus
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 3 - DNA, 558 bp, Gaussia princeps
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 4 - DNA, 579 bp, Unknown
  source: /note=“Description of Unknown EPO DNA template sequence”
atgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgc

SEQ ID NO: 5 - DNA, 741 bp, Coxsackievirus B3
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 6 - DNA, 711 bp, Unknown
  source: /note=“Description of Unknown Green Fluorescent Protein DNA template sequence”
agcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 7 - DNA, 20 bp, Artificial Sequence
  source: /note=“Description of Artificial Sequence Synthetic primer”
agatttcgttggggactggc

SEQ ID NO: 8 - DNA, 20 bp, Artificial Sequence
  source: /note=“Description of Artificial Sequence Synthetic primer”
caccgctcaggacaatcctt

SEQ ID NO: 9 - DNA, 20 bp, Artificial Sequence
  source: /note=“Description of Artificial Sequence Synthetic primer”
ctggagacgtggaggagaac

SEQ ID NO: 10 - DNA, 20 bp, Artificial Sequence
  source: /note=“Description of Artificial Sequence Synthetic primer”
ccaaaagacggcaatatggt

SEQ ID NO: 11 - DNA, 29 bp, Artificial Sequence
  source: /note=“Description of Artificial Sequence Synthetic oligonucleotide”
tttttcggctattcccaatagccgttttg

SEQ ID NO: 12 - DNA, 20 bp, Artificial Sequence
  source: /note=“Description of Artificial Sequence Synthetic primer”
ccgtatgaaggtctgagcgg

SEQ ID NO: 13 - DNA, 20 bp, Artificial Sequence
  source: /note=“Description of Artificial Sequence Synthetic primer”
cagtgtgccatagtgcagga